(19) United States
(12) Patent Application Publication
(10) Pub. No.: US 2024/0425545 A1
(43) Pub. Date: Dec. 26, 2024

(54) 25-Hydroxy-Cholest-5-En-3-Sulfate Choline and Methods for Preparing, and Uses of, Same

(71) Applicant: Durect Corporation, Cupertino, CA (US)

(72) Inventors: Andrew R. Miksztal, PALo ALTO, CA (US); Howard Sard, Arlington, MA (US); Mario Gonzalez, Winchester, MA (US); Cynthia A. Morency, Andover, MA (US); WeiQi Lin, Emerald Hills, CA (US)

(21) Appl. No.: 18/572,715

(22) PCT Filed: Jun. 24, 2022
(86) PCT No.: PCT/US2022/034959

§ 371 Date: Dec. 20, 2023

Related U.S. Application Data
(60) Provisional application No. 63/215,383, filed on Jun. 25, 2021.

(51) Int. Cl. C07J 31/00; A61K 31/575

(52) U.S. Cl. CPC C07J 31/006; A61K 31/575

(57) ABSTRACT

25HC3S choline and crystalline 25HC3S choline are described herein. Pharmaceutical formulations of 25HC3S choline such as with crystalline 25HC3S choline and methods of treating or preventing disease with same such as hypercholesterolemia, hypertriglyceridemia, and conditions related to fat accumulation and inflammation (e.g., non-alcoholic fatty liver disease (NAFLD), non-alcoholic steatohepatitis (NASH), alcoholic hepatitis, acute kidney injury (AKI), psoriasis, and atherosclerosis) are further disclosed herein. Methods for preparing 25HC3S, including crystalline 25HC3S choline, are also provided.

### INTRODUCTION

It has been shown previously that cholesterol metabolite 5-cholesten-3β-25-diol-3-sulphate (“25HC3S”) decreases lipid biosynthesis and increases cholesterol secretion and degradation, and may be useful for the treatment and prevention of hypercholesterolemia, hypertriglyceridemia, and conditions related to fat accumulation and inflammation (e.g., non-alcoholic fatty liver disease (NAFLD), non-alcoholic steatohepatitis (NASH), alcoholic hepatitis, acute kidney injury (AKI), psoriasis, and atherosclerosis).

Cholesterol is used by the body for the manufacture and repair of cell membranes, and the synthesis of steroid hormones and vitamin D, and is transformed to bile acids in the liver. There are both exogenous and endogenous sources of cholesterol. The average American consumes about 450 mg of cholesterol each day and produces an additional 500 mg to 1,000 mg in the liver and other tissues. Another source is the 500 mg to 1,000 mg of biliary cholesterol that is secreted into the intestine daily, and about 50 percent is reabsorbed (enterohepatic circulation).

High serum lipid levels (hypercholesterolemia and hypertriglyceridemia) are associated with the accumulation of cholesterol in arterial walls, and can result in NAFLD and atherosclerosis. The plaques that characterize atherosclerosis inhibit blood flow and promote clot formation, and can ultimately cause death or severe disability via heart attacks and/or stroke. A number of therapeutic agents for the treatment of hyperlipidemia have been developed and are widely prescribed by physicians. Unfortunately, only about 35% of patients are responsive to the currently available therapies.

Non-alcoholic fatty liver disease (NAFLD) is the most common liver disease in the United States. This condition is associated with obesity, type-II adult-onset diabetes, sedentary lifestyle, and diets high in fat. The earlier stage of NAFLD, fatty liver, is potentially reversible when proper treatment steps are taken. However, left unchecked, it can progress to inflammation of liver cells (non-alcoholic steatohepatitis, or NASH) which is much more difficult to treat. Without treatment, NASH can result in irreversible scarring of liver tissue (steatonecrosis), with the potential to cause cirrhosis, liver failure, and liver cancer.

Certain pharmaceutically acceptable salts, such as a sodium salt, of 25HC3S have been disclosed (e.g., U.S. Pat. No. 10,144,759 and Ogawa et al., Steroids, 74, 81-87 (2009)). Different salts of 25HC3S may bring different practical benefits and disadvantages, for instance in relation to their amenability to processing into desired pharmaceutical formulations, their clinical efficacy in addressing particular pathological indications, and the like.

Crystalline solids tend to be more favorable for processing, storage, and stability than non-crystalline solids. However, energetics may not favor the ready formation of suitable crystalline solids and polymorphism may make creating stable crystalline solids of a particular active pharmaceutical ingredient impractical.

Herein, the inventors disclose 25HC3S choline, including crystalline 25HC3S choline. It has been surprisingly found that 25HC3S choline provides better quality crystals than other salts of 25HC3S. It has also been surprisingly found that 25HC3S choline is less hygroscopic than other salts, such as 25HC3S sodium. Methods for preparing 25HC3S choline, including crystalline 25HC3S choline, are also provided.

### SUMMARY

In some aspects of the present disclosure, 25HC3S choline is provided.

In some aspects of the disclosure, crystalline 25HC3S choline is provided.

In other aspects of the disclosure, substantially pure crystalline 25HC3S choline is provided.

In other aspects of the disclosure, processes for preparing 25HC3 S choline, including crystalline 25HC3S choline, are provided.

In additional aspects of the disclosure, 25HC3S choline, including crystalline 25HC3S choline, prepared by the processes of the disclosure are provided.

In still additional aspects of the disclosure, pharmaceutical compositions comprising 25HC3S choline, including crystalline 25HC3S choline, and a pharmaceutically acceptable excipient are provided.

In further aspects of the disclosure, methods of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, and conditions related to fat accumulation and inflammation, comprising administering to a patient in need thereof an effective amount of 25HC3S choline, including crystalline 25HC3S choline, are provided.

In still further aspects of the disclosure, uses of 25HC3S choline, including crystalline 25HC3S choline, and optionally one or more pharmaceutically acceptable excipients, for (i) treating a patient in need thereof with or (ii) preventing in a patient in need thereof, one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, and conditions related to fat accumulation and inflammation, or preventing same, are provided.

In additional aspects of the disclosure, uses of 25HC3S choline, including crystalline 25HC3S choline, and optionally one or more pharmaceutically acceptable excipients, for manufacturing a medicament for (i) treating a patient with or (ii) preventing in a patient in need thereof, one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, and conditions related to fat accumulation and inflammation, or preventing same, are provided.

### BRIEF DESCRIPTION OF THE FIGURES

FIG. 1 is an x-ray powder diffraction (XRPD) diffractogram of crystalline 25HC3S choline.

FIG. 2 is a peak-picked XRPD diffractogram of crystalline 25HC3S choline.

FIG. 3 is an XRPD diffractogram overlay of crystalline 25HC3S choline before and after a dynamic vapor sorption (DVS) experiment.

FIG. 4 is indexing results for crystalline 25HC3S choline.

FIG. 5 is a differential scanning calorimetry (DSC) thermogram and a thermogravimetric analysis (TGA) thermogram for crystalline 25HC3S choline.

FIG. 6 is a H-NMR spectrum of 25HC3S choline in solution.

FIGS. 7A and 7B is a DVS isotherm of crystalline 25HC3S choline.

FIG. 8 is a DVS isotherm of crystalline 25HC3S sodium.

### DETAILED DESCRIPTION

### Crystalline 25-hydroxy-3β-cholesten-5-en-3-sulfate (25HC3S) choline

As described herein, the compound 25-hydroxy-3β-cholesten-5-en-3-sulfate (25HC3S) refers to [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl] sulfate, the compound of Formula I:

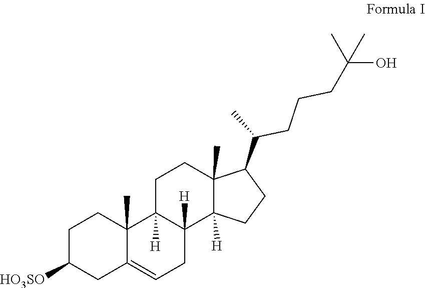

The term “25HC3S choline” means the choline salt of 25HC3S. The term “crystalline 25HC3S choline” means the crystalline choline salt of 25HC3S, i.e. the choline salt of 25HC3S in crystalline form. Choline is a quaternary ammonium compound. It is often available as choline hydroxide. 25HC3S choline has the following structure:

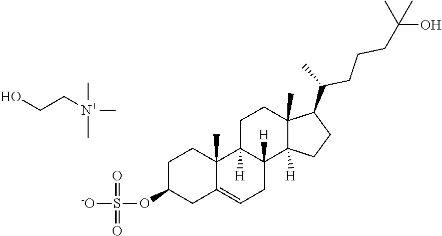

Choline is an essential nutrient that is naturally present in some foods and available as a dietary supplement. Choline is a source of methyl groups needed for many steps in metabolism. The body needs choline to synthesize phosphatidyicholine and sphingomyelin, two phospholipids associated with cell membranes, and to produce the neurotransmitter acetylcholine. Choline deficiency is associated with undesirable clinical indications, including the manifestation of the conditions described herein.

Crystalline 25HC3S choline is readily analyzed or characterized by x-ray powder diffraction. An x-ray powder diffraction pattern is an x-y graph with °2θ(diffraction angle) on the x-axis and intensity on the y-axis. The x-axis can also be in the form of d-spacings which is related to the diffraction angle via the Bragg's law whereby 2dsinθ=nλ where d is the d-spacing and k is the wavelength of the incident x-ray wave. The pattern contains peaks which may be used to characterize crystalline 25HC3S choline. Unless otherwise specified, peaks are referred to by their position on the x-axis and not their y-axis intensity. It can also occur that due to sample orientation, a peak that is present in one sample on one instrument may not be present in another sample taken on a different instrument due to the orientation of the sample with respect to the instrument.

The data from x-ray powder diffraction may be used in multiple ways to characterize crystalline forms. For example, the entire x-ray powder diffraction pattern output from a diffractometer may be used to characterize crystalline 25HC3S choline. A smaller subset of such data, however, may also be, and typically is, suitable for characterizing crystalline 25HC3S choline. For example, a collection of one or more peaks from such a pattern may be used to characterize crystalline 25HC3S choline. In the present application, all reported peak values are in °2θ with Cu-Kα radiation, e.g., as set forth in Example 24 and Example 25. Indeed, often even a single x-ray powder diffraction peak may be used to characterize such a crystalline form. When crystalline 25HC3S choline herein is characterized by “one or more peaks” of an x-ray powder diffraction pattern and such peaks are listed, what is generally meant is that any combination of the peaks listed may be used to characterize crystalline 25HC3S choline. Further, the fact that other peaks are present in the x-ray powder diffraction pattern, generally does not negate or otherwise limit that characterization.

In addition to the variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, orientation and size, particle size, moisture content, solvent content, instrument and experimental parameters, data analysis software). For example, samples of the same crystalline material prepared under different conditions may yield slightly different diffractograms, and different x-ray instruments may operate using different parameters, and these may lead to slightly different diffraction patterns from the same crystalline solid.

Due to such sources of variability, it is common to recite x-ray diffraction peaks using the word “about” prior to the peak value in °2θ. For purposes of data reported herein, that value is generally ±0.2° 2θ. This generally means that on a well-maintained instrument one would expect the variability in peak measurement to be ±0.2° 2θ on the same instrument. Unless specified otherwise, x-ray powder diffraction peaks cited herein are generally reported with this variability of 0.2° 2θ and are generally intended to be reported with such a variability whenever disclosed herein whether the word “about” is present or not, however, variability may, in some instances, be higher depending on instrumentation conditions. Furthermore, in additional embodiments of the invention, the variability in a quoted peak value or grouping of quoted peak values in °2θ is +0.1°2θ, or even ±0.05° 2θ, rather than ±0.2° 2θ.

The x-ray powder diffraction data from crystalline 25HC3S choline may be used to index the corresponding unit cell. “Indexing,” as used herein, generally refers to the process of determining the size and shape of the crystallographic unit cell given the peak positions in a diffraction pattern. The term gets its name from the assignment of Miller index labels to individual peaks. For example, if all of the peaks in a pattern are indexed by a single unit cell, this can be strong evidence that the sample contains a single crystalline phase. Given the indexing solution, the unit cell volume may be calculated directly and can be useful to determine their solvation states. Indexing may also be a description of a crystalline form and provides a concise summary of all available peak positions for that phase at a particular thermodynamic state point.

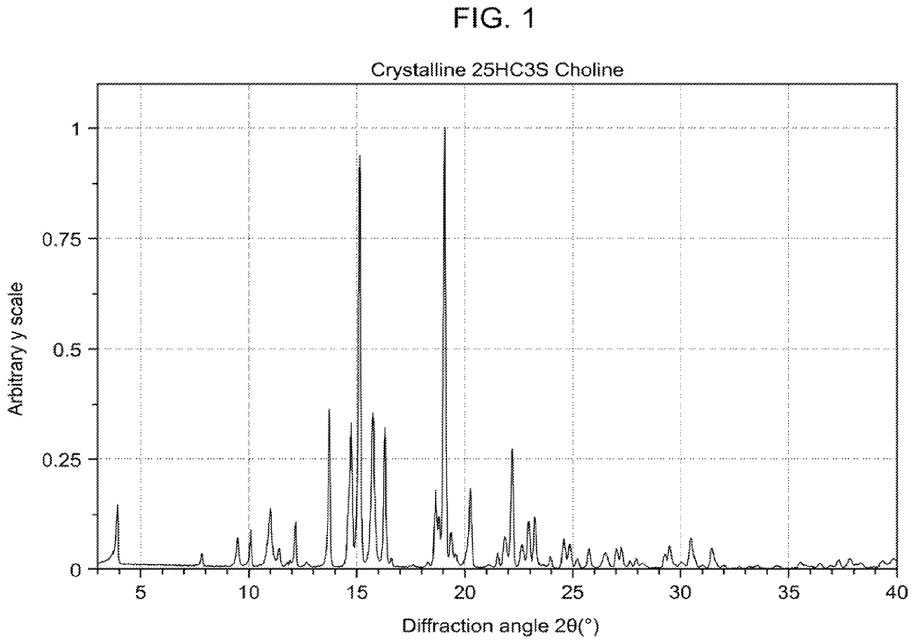

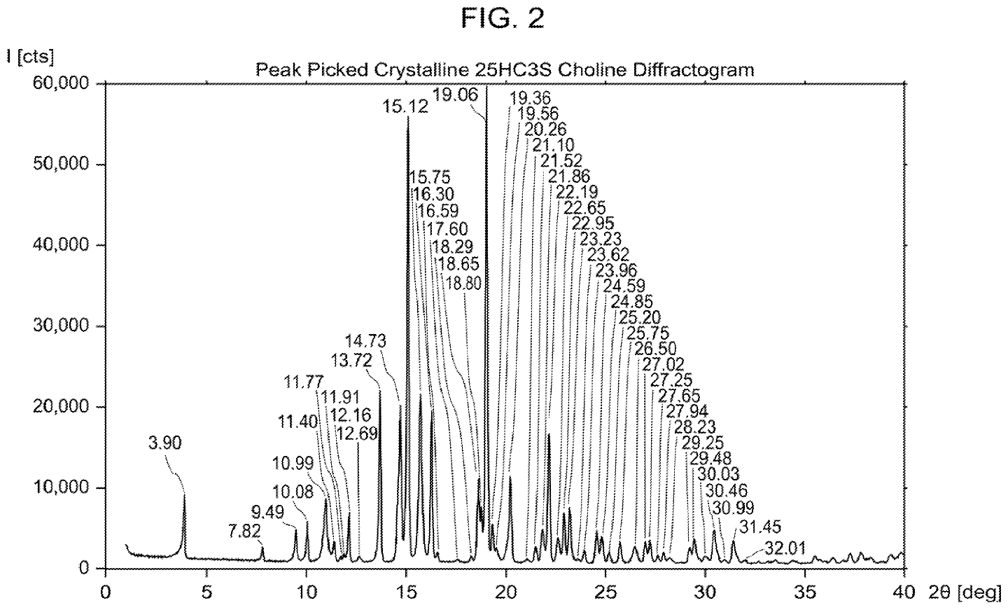

25HC3S choline, including crystalline 25HC3S choline, may be prepared as set forth in Example 28. An x-ray powder diffraction pattern of crystalline 25HC3S choline can be found in FIG. 1 and a peak-picked version in FIG. 2. Table 1 shows picked peaks from FIG. 2.

TABLE 1
Peaks of Crystalline 25HC3S Choline of FIG. 2

| °2θ | d space (Å) | Intensity (%) |
|---|---|---|
| 3.90 ± 0.20 | 22.638 ± 1.160 | 15 |
| 7.82 ± 0.20 | 11.296 ± 0.288 | 4 |
| 9.49 ± 0.20 | 9.312 ± 0.196 | 8 |
| 10.08 ± 0.20 | 8.768 ± 0.174 | 10 |
| 10.99 ± 0.20 | 8.044 ± 0.146 | 15 |
| 11.40 ± 0.20 | 7.756 ± 0.136 | 5 |
| 11.77 ± 0.20 | 7.513 ± 0.127 | 2 |
| 11.91 ± 0.20 | 7.425 ± 0.124 | 3 |
| 12.16 ± 0.20 | 7.273 ± 0.119 | 11 |
| 12.69 ± 0.20 | 6.970 ± 0.109 | 2 |
| 13.72 ± 0.20 | 6.449 ± 0.094 | 37 |
| 14.73 ± 0.20 | 6.009 ± 0.081 | 33 |
| 15.12 ± 0.20 | 5.855 ± 0.077 | 93 |
| 15.75 ± 0.20 | 5.622 ± 0.071 | 36 |
| 16.30 ± 0.20 | 5.434 ± 0.066 | 33 |
| 16.59 ± 0.20 | 5.339 ± 0.064 | 3 |
| 17.60 ± 0.20 | 5.035 ± 0.057 | 2 |
| 18.29 ± 0.20 | 4.847 ± 0.053 | 2 |
| 18.65 ± 0.20 | 4.754 ± 0.051 | 19 |
| 18.80 ± 0.20 | 4.716 ± 0.050 | 13 |
| 19.06 ± 0.20 | 4.653 ± 0.048 | 100 |
| 19.36 ± 0.20 | 4.581 ± 0.047 | 9 |
| 19.56 ± 0.20 | 4.535 ± 0.046 | 4 |
| 20.26 ± 0.20 | 4.380 ± 0.043 | 19 |
| 21.10 ± 0.20 | 4.207 ± 0.039 | 2 |
| 21.52 ± 0.20 | 4.126 ± 0.038 | 4 |
| 21.86 ± 0.20 | 4.063 ± 0.037 | 8 |
| 22.19 ± 0.20 | 4.003 ± 0.036 | 28 |
| 22.65 ± 0.20 | 3.923 ± 0.034 | 6 |
| 22.95 ± 0.20 | 3.872 ± 0.033 | 12 |
| 23.23 ± 0.20 | 3.826 ± 0.032 | 13 |
| 23.62 ± 0.20 | 3.764 ± 0.031 | 2 |
| 23.96 ± 0.20 | 3.711 ± 0.031 | 4 |
| 24.59 ± 0.20 | 3.617 ± 0.029 | 8 |
| 24.85 ± 0.20 | 3.580 ± 0.028 | 6 |
| 25.20 ± 0.20 | 3.531 ± 0.028 | 3 |
| 25.75 ± 0.20 | 3.457 ± 0.026 | 5 |
| 26.50 ± 0.20 | 3.361 ± 0.025 | 4 |
| 27.02 ± 0.20 | 3.297 ± 0.024 | 5 |
| 27.25 ± 0.20 | 3.270 ± 0.024 | 6 |
| 27.65 ± 0.20 | 3.224 ± 0.023 | 3 |
| 27.94 ± 0.20 | 3.191 ± 0.022 | 3 |
| 28.23 ± 0.20 | 3.159 ± 0.022 | 2 |
| 29.25 ± 0.20 | 3.051 ± 0.020 | 4 |
| 29.48 ± 0.20 | 3.028 ± 0.020 | 6 |
| 30.03 ± 0.20 | 2.973 ± 0.019 | 2 |
| 30.46 ± 0.20 | 2.932 ± 0.019 | 8 |
| 30.99 ± 0.20 | 2.883 ± 0.018 | 2 |
| 31.45 ± 0.20 | 2.842 ± 0.018 | 6 |
| 32.01 ± 0.20 | 2.794 ± 0.017 | 2 |

Crystalline 25HC3S choline may be characterized by various analytical techniques, including by x-ray powder diffraction. The x-ray powder diffraction pattern of crystalline 25HC3S choline or portions thereof, may be used to identify crystalline 25HC3S choline. Crystalline 25HC3S choline contains various x-ray powder diffraction peaks which alone or together may help identify the presence of crystalline 25HC3S choline.

In some cases, crystalline 25HC3S choline may be characterized by an x-ray powder diffraction pattern having a peak at about 3.9°2θ. In these and other cases, crystalline 25HC3S choline may be further characterized by one or more peaks at about 7.8° 2θ, about 9.5° 2θ, about 10.1°2θ, about 11.0020, about 12.2° 2θ, about 13.7° 2θ, about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having a peak at about 7.8° 2θ. In these and other cases, crystalline 25HC3S choline may be further characterized by one or more peaks at about 3.9° 2θ, about 9.5° 2θ, about 10.1°2θ, about 11.0020, about 12.2° 2θ, about 13.7° 2θ, about 14.7° 2θ, about 15.1° 2θ, about 15.8° 2θ, about 16.3° 2θ, and about 19.1°2θ.

In some cases, crystalline 25HC3S choline may be characterized by an x-ray powder diffraction pattern having a peak at about 9.5° 2θ. In these and other cases, crystalline 25HC3S choline may be further characterized by one or more peaks at about 3.9° 2θ, about 7.8° 2θ, about 10.1°2θ, about 11.0020, about 12.2° 2θ, about 13.7° 2θ, about 14.7° 2θ, about 15.1° 2θ, about 15.8° 2θ, about 16.3° 2θ, and about 19.1°2θ.

In some cases, crystalline 25HC3S choline may be characterized by an x-ray powder diffraction pattern having a peak at about 10.1°2θ. In these and other cases, crystalline 25HC3S choline may be further characterized by one or more peaks at about 3.9° 2θ, about 7.8° 2θ, about 9.5° 2θ, about 11.0020, about 12.2°2θ, about 13.7°2θ, about 14.7°2θ, about 15.1°2θ, about 15.8° 2θ, about 16.3°2θ, and about 19.1° 2θ.

In some cases, crystalline 25HC3S choline may be characterized by an x-ray powder diffraction pattern having a peak at about 11.0°2θ. In these and other cases, crystalline 25HC3S choline may be further characterized by an x-ray powder diffraction pattern having one or more peaks at about 3.9° 2θ, about 7.8°2θ, about 9.5°2θ, about 10.1°2θ, about 12.2°2θ, about 13.7° 2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 12.2°2θ. In these and other cases, crystalline 25HC3S choline may be further characterized by an x-ray powder diffraction pattern having one or more peaks at about 3.9° 2θ, about 7.8°2θ, about 9.5°2θ, about 10.1°2θ, about 11.0°2θ, about 13.7° 2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 13.7°2θ. In these and other cases, crystalline 25HC3S choline may be further characterized by an x-ray powder diffraction pattern having one or more peaks at about 3.9° 2θ, about 7.8°2θ, about 9.5°2θ, about 10.1°2θ, about 11.0°2θ, about 12.2° 2θ, about 14.7°2θ, about 15.1°2θ, about 15.8° 2θ, about 16.3° 2θ, and about 19.1°2θ.

In some cases, crystalline 25HC3S choline may be characterized by an x-ray powder diffraction pattern having a peak at about 14.7°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2° 2θ, about 13.7°2θ, about 15.1°2θ, about 15.8° 2θ, about 16.3° 2θ, and about 19.1°2θ.

In some cases, crystalline 25HC3S choline may be characterized by an x-ray powder diffraction pattern having a peak at about 15.1°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2° 2θ, about 13.7°2θ, about 14.7°2θ, about 15.8° 2θ, about 16.3° 2θ, and about 19.1°2θ.

In some cases, crystalline 25HC3S choline may be characterized by an x-ray powder diffraction pattern having a peak at about 15.8° 2θ. In these and other cases, crystalline 25HC3S choline may be further characterized by an x-ray powder diffraction pattern having one or more peaks at about 3.9° 2θ, about 7.8° 2θ, about 9.5°2θ, about 10.1°2θ, about 11.0°2θ, about 12.2° 2θ, about 13.7°2θ, about 14.7°2θ, about 15.1°2θ, about 16.3° 2θ, and about 19.1° 2θ.

In some cases, crystalline 25HC3S choline may be characterized by an x-ray powder diffraction pattern having a peak at about 16.3°2θ. In these and other cases, crystalline 25HC3S choline may be further characterized by an x-ray powder diffraction pattern having one or more peaks at about 3.9°2θ, about 7.8° 2θ, about 9.5°2θ, about 10.1°2θ, about 11.0°2θ, about 12.2° 2θ, about 13.7°2θ, about 14.7°2θ, about 15.1°2θ, about 15.8° 2θ, and about 19.1°2θ.

In some cases, crystalline 25HC3S choline may be characterized by an x-ray powder diffraction pattern having a peak at about 19.1°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2° 2θ, about 13.7°2θ, about 14.7°2θ, about 15.1° 2θ, about 15.8° 2θ, and about 16.3° 2θ.

In some cases, crystalline 25HC3S choline may be characterized by an x-ray powder diffraction pattern having peaks at about 3.9° 2θ and about 7.8°2θ. In these and other cases, crystalline 25HC3S choline may be further characterized by an x-ray powder diffraction pattern having one or more peaks at about 9.5°2θ, about 10.1° 2θ, about 11,0°2θ, about 12.2°2θ, about 13.7° 2θ, about 14.7°2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, and about 9.5° 2θ. In these and other cases, crystalline 25HC3S choline may be further characterized by an x-ray powder diffraction pattern having one or more peaks at about 10.1° 2θ, about 11.0°2θ, about 12.2° 2θ, about 13.7° 2θ, about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, about 9.5° 2θ, and about 10.1°2θ. In these and other cases, crystalline 25HC3S choline may be further characterized by an x-ray powder diffraction pattern having one or more peaks at about 11.0° 2θ, about 12.2° 2θ, about 13.7° 2θ, about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, about 9.5° 2θ, about 10.1°2θ, and about 11.0020. In these and other cases, crystalline 25HC3S choline may be further characterized by an x-ray powder diffraction pattern having one or more peaks at about 12.2° 2θ, about 13.7° 2θ, about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, about 9.5° 2θ, about 10.1°2θ, about 11.0020, and about 12.2° 2θ. In these and other cases, crystalline 25HC3S choline may be further characterized by an x-ray powder diffraction pattern having one or more peaks at about 13.7° 2θ, about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, about 9.5° 2θ, about 10.1°2θ, about 11.0020, about 12.2° 2θ, and about 13.7° 2θ. In these and other cases, crystalline 25HC3S choline may be further characterized by an x-ray powder diffraction pattern having one or more peaks at about 14.7° 2θ, about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9° 2θ, about 7.8° 2θ, about 9.5° 2θ, about 10.1°2θ, about 11.0020, about 12.2° 2θ, about 13.7° 2θ, and about 14.7° 2θ. In these and other cases, crystalline 25HC3S choline may be further characterized by an x-ray powder diffraction pattern having one or more peaks at about 15.1°2θ, about 15.8° 2θ, about 16.3° 2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0020, about 12.2° 2θ, about 13.7° 2θ, about 14.7° 2θ, and about 15.1°2θ. In these and other cases, crystalline 25HC3 S choline may be further characterized by an x-ray powder diffraction pattern having one or more peaks at about 15.8° 2θ, about 16.3° 2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0020, about 12.2° 2θ, about 13.7° 2θ, about 14.7° 2θ, about 15.1° 2θ, and about 15.8° 2θ. In these and other cases, crystalline 25HC3S choline may be further characterized by an x-ray powder diffraction pattern having one or more peaks at about 16.3° 2θ and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0020, about 12.2° 2θ, about 13.7° 2θ, about 14.7° 2θ, about 15.1°2θ, about 15.8° 2θ, and about 16.3° 2θ. In these and other cases, crystalline 25HC3S choline may be further characterized by an x-ray powder diffraction pattern having a peak at about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0° 2θ, about 12.2° 2θ, about 13.7° 2θ, about 14.7° 2θ, about 15.1° 2θ, about 15.8° 2θ, about 16.3° 2θ, and about 19.1°2θ.

In some cases, crystalline 25HC3S choline may be characterized by an x-ray powder diffraction pattern having one or more peaks at about 7.8°2θ, about 9.5° 2θ, about 10.1°2θ, about 11.0020, about 12.2° 2θ, about 13.7° 2θ, about 14.7° 2θ, about 15.1° 2θ, about 15.8° 2θ, about 16.3° 2θ, and about 19.1°2θ.

In some cases, crystalline 25HC3S choline may be characterized by an x-ray powder diffraction pattern having one or more peaks at about 9.5°2θ, about 10.1° 2θ, about 11.0°2θ, about 12.2° 2θ, about 13.7°2θ, about 14.7°2θ, about 15.1°2θ, about 15.8°2θ, about 16.3°2θ, and about 19.1° 2θ.

In some cases, crystalline 25HC3S choline may be characterized by an x-ray powder diffraction pattern having one or more peaks at about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 11.0°2θ, about 12.2°2θ, about 13.7° 2θ, about 14.7°2θ, about 15.1°2θ, about 15.8° 2θ, about 16.3° 2θ, and about 19.1°2θ.

In some cases, crystalline 25HC3S choline may be characterized by an x-ray powder diffraction pattern having one or more peaks at about 12.2°2θ, about 13.7°2θ, about 14.7°2θ, about 15.1°2θ, about 15.8° 2θ, about 16.3° 2θ, and about 19.1° 2θ.

In some cases, crystalline 25HC3S choline may be characterized by an x-ray powder diffraction pattern having one or more peaks at about 13.7°2θ, about 14.7° 2θ, about 15.1° 2θ, about 15.8° 2θ, about 16.3°2θ, and about 19.1°2θ.

In some cases, crystalline 25HC3S choline may be characterized by an x-ray powder diffraction pattern having one or more peaks at about 14.7° 2θ, about 15.1°2θ, about 15.8° 2θ, about 16.3°2θ, and about 19.1°2θ.

In some cases, crystalline 25HC3S choline may be characterized by an x-ray powder diffraction pattern having one or more peaks at about 15.1°2θ, about 15.8° 2θ, about 16.3° 2θ, and about 19.1°2θ.

In some cases, crystalline 25HC3S choline may be characterized by an x-ray powder diffraction pattern having one or more peaks at about 15.8° 2θ, about 16.3° 2θ, and about 19.1°2θ.

In some cases, crystalline 25HC3S choline may be characterized by an x-ray powder diffraction pattern having one or more peaks at 16.3° 2θ and about 19.1°2θ.

In some cases, crystalline 25HC3S choline may be characterized by an x-ray powder diffraction pattern having a peak at about 19.1°2θ.

In some cases, crystalline 25HC3S choline may be characterized by an x-ray powder diffraction pattern having substantially the same pattern as that found in FIG. 1.

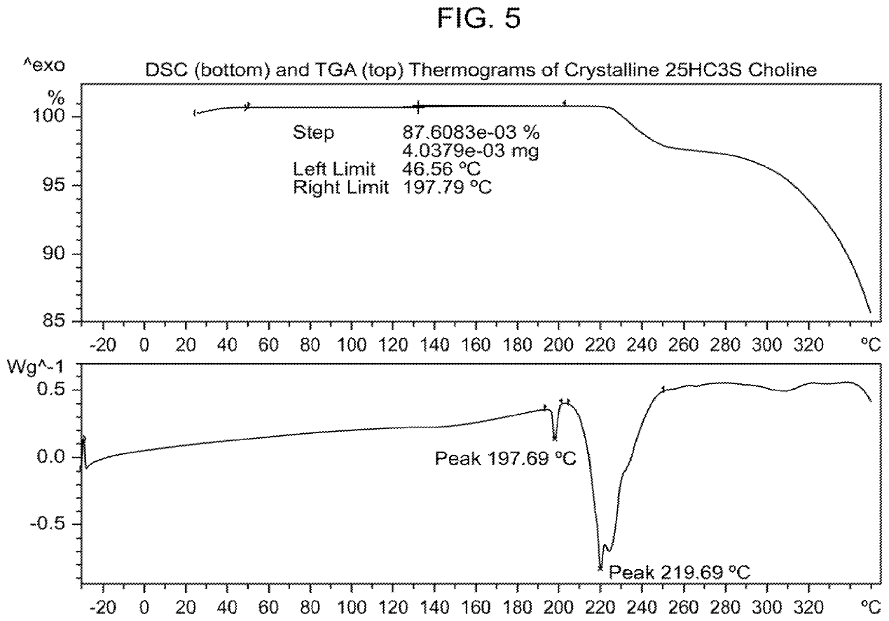

A DSC thermogram of crystalline 25HC3S choline indicated endothermic peaks at about 198° C. and about 220° C. The TGA thermogram of FIG. 5 indicates that there is negligible weight loss up to 198° C. Exemplary means for measuring any characterizing DSC endothermic peaks, and any characterizing DSC thermograms, are set out in Examples 21 and 23. In the context of DSC measurements, there is also variability and the term “about” means±1° C., and such variability is to be understood whether a DSC measurement is prefaced by “about” or not unless specified otherwise.

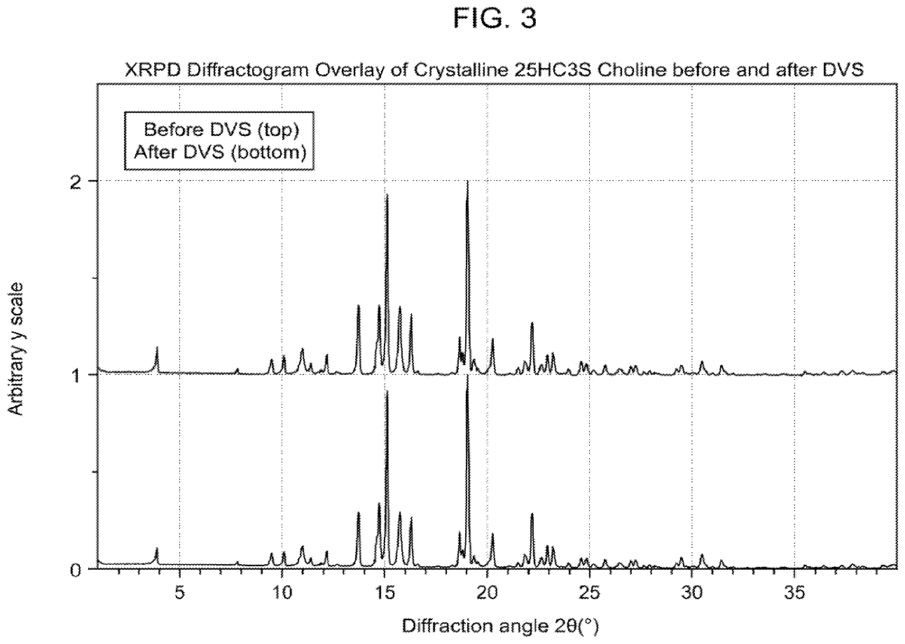

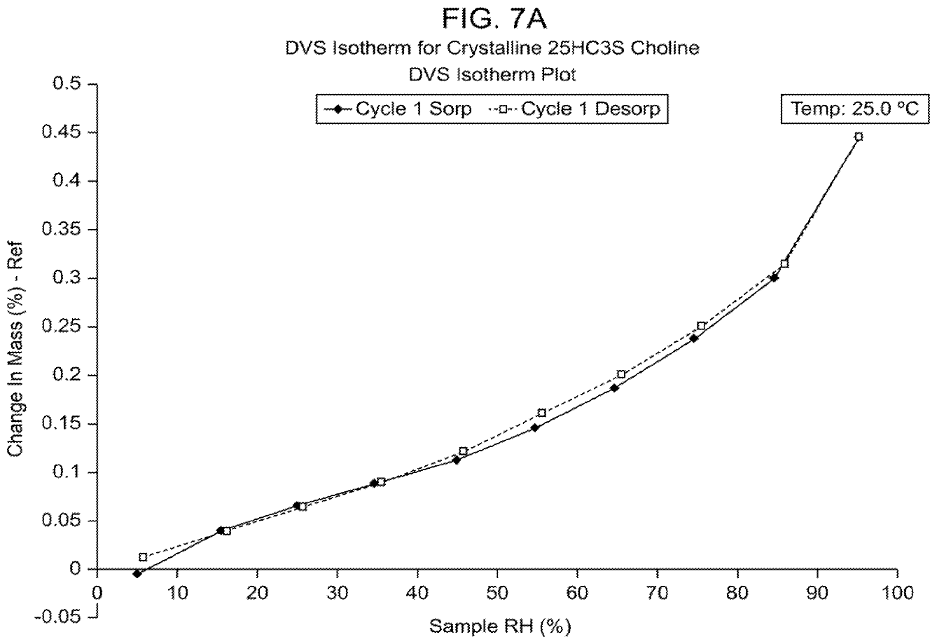

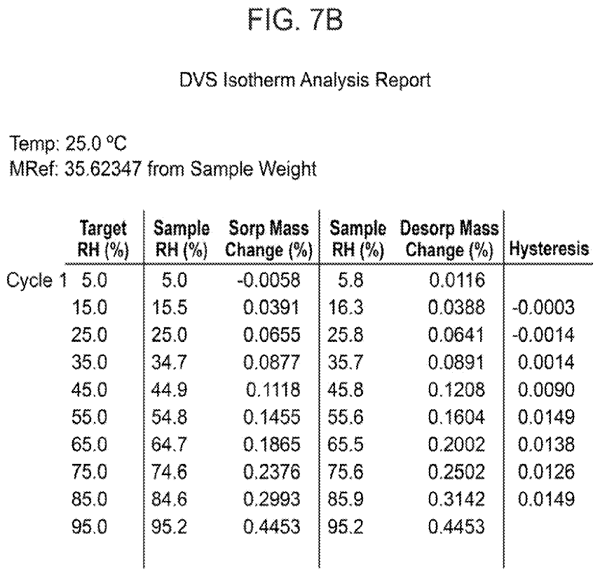

Without being bound by theory, it is believed that crystalline 25HC3S choline is an anhydrate, meaning that there is no water of crystallization in the unit cell. This does not preclude the possibility of other water being present in a solid comprising crystalline 25HC3S choline. In addition, crystalline 25HC3S choline is not appreciably hygroscopic up to about 95% relative humidity, increasing in weight by only about 0.5% up to this relative humidity as evidenced by a dynamic vapor sorption experiment according to Example 26 and whose results are shown in FIG. 7. Further, the x-ray powder diffraction pattern of crystalline 25HC3S choline does not appreciably change after DVS as shown in FIG. 3. Only a 0.5% weight gain was observed on going from 5% to 95% relative humidity and a 0.5% weight loss was observed on the return to 5% relative humidity indicating no hysteresis.

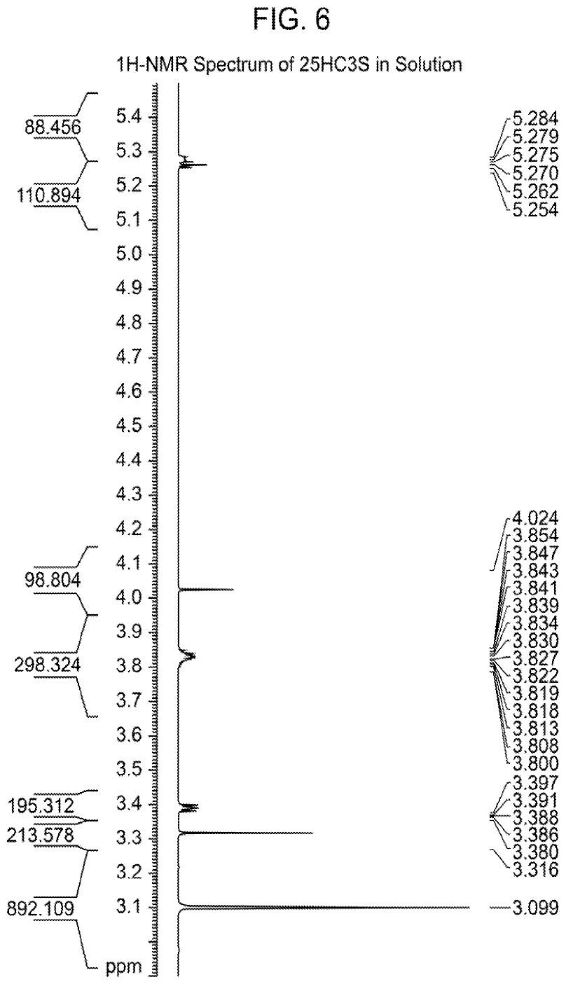

Such low hygroscopicity indicates good stability under such stresses, which, as further discussed elsewhere herein, may make it suitably stable for pharmaceutical processing. Indeed, the present disclosure thus further includes stable crystalline 25HC3S choline. Such stability includes, for example, sufficiently stable crystalline 25HC3S choline to be formulated for patient delivery. The ¹H-NMR spectrum is, other than a peak at 5.3 ppm, consistent with structure as seen in FIG. 6.

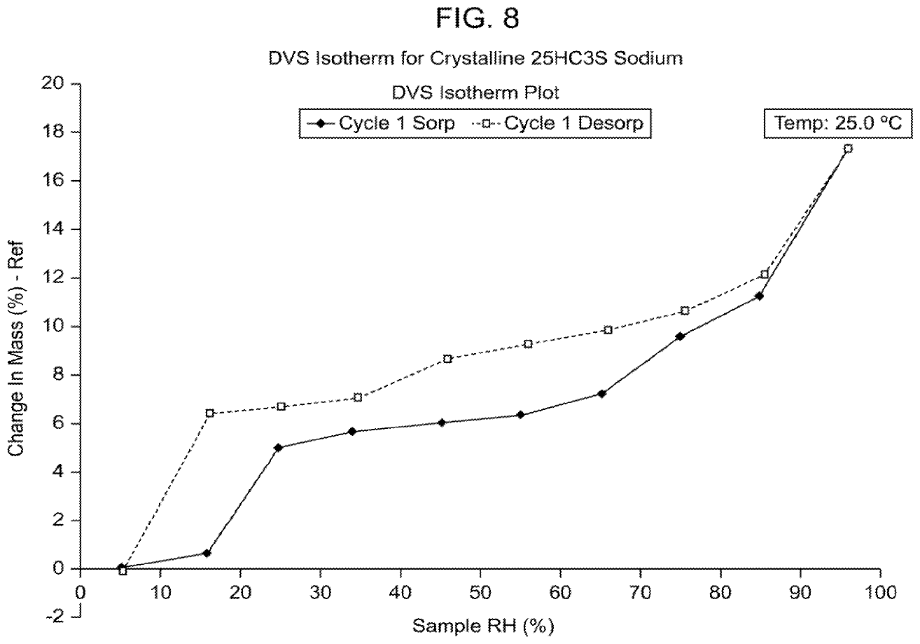

The choline salt has the additional advantage over the sodium salt and some other salts of 25HC3S in that the choline counterion has additional beneficial properties. For example, choline is an essential nutrient and lack of choline has been indicated as a cause of fat and cholesterol build up in the liver. Further, 25HC3S choline forms crystals of better quality and diffraction than those of the prior art. Lastly, crystalline 25HC3S choline is less hygroscopic, and thus more physically stable than, for example, crystalline 25HC3S sodium. Crystalline 25HC3S sodium stabilizes as a hydrate when exposed to humid conditions. In particular, monohydrates, dihydrates, and variable hydrates of crystalline 25HC3S sodium have been prepared. Form I, a hydrate, has been found to be hygroscopic and may form a liquid crystal at high water activities (e.g., above 0.73). Another hydrate, Form II, is stable at relative humidities between about 21% and about 30%. FIG. 8 shows a DVS isotherm of a manufactured batch of crystalline 25HC3S sodium containing both Form I and Form II and shows significant water uptake until about 95% relative humidity. By comparison, under conditions going up to about 95% relative humidity, only about 0.5% water by weight is absorbed indicating crystalline 25HC3S choline is stable as an anhydrate.

Substantially pure crystalline 25HC3S choline is further disclosed. “Substantially pure,” as described herein, generally refers to a form herein that is present without any appreciable amounts, other than potentially trace levels of other forms of 25HC3S choline. Examples of trace levels include not more than about 10%, 5%, 2%, 1.5%, 1%, 0.5%, 0.25%, 0.1%, or less in total relative to the total amount (based on weight) of 25HC3S choline present.

Processes of preparing 25HC3S choline are further described herein. In some cases, one may first prepare a sodium salt of 25HC3S. Examples of such preparation are set forth herein. The sodium salt of 25HC3S, which may be crystalline, may be converted into, for example, a triethylammonium salt as described in Example 27. The triethylammonium salt may then be used to create 25HC3S choline as set forth in Example 28.

The preparation of the triethylammonium salt of 25HC3S may be accomplished, for example, by passing a mixture of triethylammonium chloride and triethylamine through a column and treating with a solvent such as an alcohol until neutral pH. Separately, crystalline 25HC3S sodium may be dissolved in a solvent such as an alcohol. The solution may then be passed through the same column previously exposed to triethylamine and combined with the triethylammonium solution. Isolating resulting solids such as under vacuum or by drying may then provide crystalline 25HC3S triethylammonium salt which may be homogenized, for example, with a mortar and pestle. A suitable alcohol for this process includes methanol.

25HC3S choline, including crystalline 25HC3S choline, may be prepared by starting with 25HC3S sodium, converting to a second salt of 25HC3S such as the triethylammonium salt, and then converting that second salt of 25HC3S to 25HC3S choline, including crystalline 25HC3S choline. The preparation of crystalline 25HC3S choline may be accomplished by preparing a suspension of a triethylammonium salt of 25HC3S in a suitable solvent such as acetonitrile and treating with a choline source such as aqueous choline hydroxide to form 25HC3S choline including crystalline 25HC3S choline. The 25HC3S choline may be purified such as by rinsing with a suitable solvent. Additional processing such as drying under vacuum or otherwise may also be performed. The disclosure further includes crystalline 25HC3S choline made by the processes described herein.

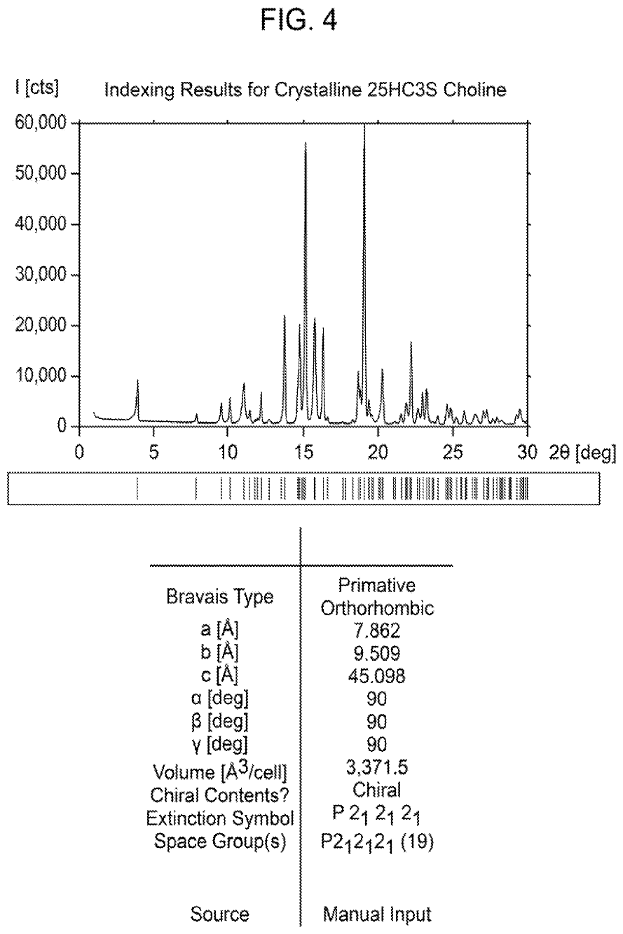

The x-ray powder diffraction pattern of crystalline 25HC3S choline was successfully indexed, indicating the pattern represents a single crystalline phase with the results set forth in FIG. 4. The indexing result reveals crystalline 25HC3S choline to have an orthorhombic cell with a cell volume of 3371.5 Å³, consistent with an anhydrous form. Several cell parameters are set forth in Table 2 below.

TABLE 2
Indexing Summary for Crystalline 25HC3S Choline

| | Primitive |
|---|---|
| Bravais Type | Orthorhombic |
| a[Å] | 7.862 |
| b[Å] | 9.509 |
| c[Å] | 45.098 |
| α | 90 |
| β | 90 |
| γ | 90 |
| Volume [Å³/cell] | 3,371.5 |
| Chiral Contents? | Chiral |
| Extinction Symbol | P 2₁ 2₁ 2₁ |
| Space Group(s) | P2₁2₁2₁ (19) |
| Source | Manual Input |

The present disclosure also relates to pharmaceutical compositions containing 25HC3S choline, including crystalline 25HC3S choline, as disclosed herein. Such pharmaceutical compositions are comprised of one or more pharmaceutically acceptable excipients and 25HC3S choline, including crystalline 25HC3S choline. Such pharmaceutical compositions may be administered orally or configured to be delivered as any effective conventional dosage forms, including, for example, immediate, slow and timed-release oral preparations, parenterally, topically, nasally, ophthalmically, optically, sublingually, rectally, vaginally, and the like.

As discussed elsewhere herein, and demonstrated in the Examples, the 25HC3S choline of the disclosure has surprisingly low hygroscopicity, including in comparison to other salt forms of 25HC3 S. The 25HC3 S choline can therefore advantageously manufactured and may be utilized in the preparation of pharmaceutical formulations, and particularly in the preparation of dosage forms for oral administration (e.g. solid dosage forms, such as tablets, capsules (each of which includes immediate release, sustained release or timed release formulations), pills, powders, or granules.

Still further, the 25HC3S choline of the disclosure also advantageously provides supplementary choline to patients suffering from the conditions targeted by the 25HC3S. As discussed elsewhere herein, choline deficiency can contribute to these conditions and it may be advantageous to provide choline alongside the 25HC3S in the course of therapy. Hence, the 25HC3S choline surprisingly and beneficially combines advantageous salt form properties, contributing for instance to the preparation of oral dosage forms particularly well suited for treating certain conditions, along with intrinsic ability beneficially to provide choline supplementation in course of conducting methods of treatment using the said oral dosage forms.

The present disclosure further includes methods and uses for treating and/or preventing diseases (e.g., in humans) such as one or more of hypercholesterolemia, hypertriglyceridemia, and conditions related to fat accumulation and inflammation (e.g., non-alcoholic fatty liver disease (NAFLD), non-alcoholic steatohepatitis (NASH), alcoholic hepatitis, acute kidney injury (AKI), psoriasis, and atherosclerosis) with effective amounts 25HC3S choline, including crystalline 25HC3S choline and/or pharmaceutical compositions comprising crystalline 25HC3S choline of the present disclosure.

### Methods for preparing 25-hydroxy-cholesten-5-en-3-sulfate (25HC3S)

Various methods for preparing 25-hydroxy-cholesten-5-en-3-sulfate, such as 25-hydroxy-3β-cholesten-5-en-3-sulfate (25HC3S), are described herein. There are also other methods of making 25HC3S not described herein. Although many of the teachings herein involve a sulfate in the 3p position, the teachings of the present disclosure are also generally applicable to a sulfate in the 3a position. The components used in each step of the subject methods for preparing 25-hydroxy-3β-cholesten-5-en-3-sulfate described herein may be a purified composition or a crude composition as desired. The term “purified” is used in its conventional sense to refer to a composition where at least some isolation or purification process has been conducted, such as for example, filtration or aqueous workup of a reaction mixture. In certain instances, purification includes at least one of liquid chromatography, recrystallization, distillation (e.g., azeotropic distillation) and other type of compound purification. For example, compounds as described herein may be purified by chromatographic means, such as high performance liquid chromatography (HPLC), supercritical fluid chromatography (SFC), thin layer chromatography, flash column chromatography and ion exchange chromatography. Any suitable stationary phase can be used, including normal and reversed phases as well as ionic resins. Mobile phases may be chosen from polar solvents and non-polar solvents. In some cases, the mobile phase includes a polar solvent. In some cases, the polar solvent is chosen from chloroform, dichloromethane, tetrahydrofuran, dichloroethane, acetone, dioxane, ethyl acetate, dimethylsulfoxide, aniline, diethylamine, nitromethane, acetonitrile, pyridine, isopropanol, ethanol, methanol, ethylene glycol, acetic acid and water. In some cases, the mobile phase includes a non-polar solvent. In some cases, the non-polar solvent is chosen from diethyl ether, toluene, benzene, pentane, hexanes, cyclohexane, petroleum ether and carbon tetrachloride. See, e.g., Introduction to Modern Liquid Chromatography, 2nd Edition, ed. L. R. Snyder and J. J. Kirkland, John Wiley and Sons, 1979; and Thin Layer Chromatography, ed. E. Stahl, Springer-Verlag, New York, 1969.

In some cases, a reaction mixture is used in a subsequent step in the methods described herein as a crude mixture where no purification or other workup of the reaction mixture has been conducted. In certain instances, the crude mixture includes the compound of interest in sufficient purity such as where the reaction mixture includes the compound of interest in a purity of 70% or greater, such as 75% or greater, such as 80% or greater, such as 85% or greater, such as 90% or greater, such as 95% or greater, such as 97% or greater, such as 99% or greater, such as 99.5% or greater, such as 99.9% or greater, such as 99.99% or greater and including 99.999% or greater, relative to the crude reaction mixture (apart from solvent when present), as determined by chromatography (e.g., HPLC or SFC), nuclear magnetic resonance spectroscopy (e.g., ¹H NMR or ¹³C NMR) or a combination thereof. In some cases, the compound of interest is present in the reaction mixture in an amount that is 30% by weight or greater relative to the crude reaction mixture (apart from solvent when present), such as 40% by weight or greater, such as 50% by weight or greater, such as 60% by weight or greater, such as 70% by weight or greater, such as 75% by weight or greater, such as by 80% by weight or greater, such as 85% by weight or greater, such as 90% by weight or greater, such as 95% by weight or greater, such as 97% by weight or greater, such as 99% by weight or greater, such as 99.5% by weight or greater, such as 99.9% by weight or greater, such as 99.99% by weight or greater and including 99.999% by weight or greater relative to the crude reaction mixture, and may range from 5% by weight to 99.999% by weight, such as 30% by weight to 99.99% by weight, 40% by weight to 99.9% by weight, 50% by weight to 99% by weight, 70% by weight to 95% by weight, 75% by weight to 90% by weight, 80% by weight to 99% by weight, or 80% by weight to 95% by weight. In some cases, the compound of interest is present at 30 mol % or greater in the crude reaction mixture (apart from solvent when present), such as 40 mol % or greater, such as 50 mol % or greater, such as 60 mol % or greater, such as 70 mol % or greater, such as 75 mol % or greater, such as by 80 mol % or greater, such as 85 mol % or greater, such as 90 mol % or greater, such as 95 mol % or greater, such as 97 mol % or greater, such as 99 mol % or greater, such as 99.5 mol % or greater, such as 99.9 mol % or greater, such as 99.99 mol % or greater and including 99.999 mol % or greater relative to the crude reaction mixture, and may range from 30 mol % to 99.999 mol %, such as 50 mol % to 99 mol %, 70 mol % to 95 mol %, 75 mol % to 90 mol %, 80 mol % to 99 mol %, or 80 mol % to 95 mol %.

Methods for preparing a metal salt of 25-hydroxy-3β-cholesten-5-en-3-sulfate ([(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl] sulfate metal salt) according to the present disclosure include contacting 25-hydroxy-(3β)-cholest-5-en-3-ol with a sulfating agent to produce a 25-hydroxy-(3β)-cholest-5-en-3-sulfate organic cationic salt; and contacting the 25-hydroxy-(33)-cholest-5-en-3-sulfate organic cationic salt with at least one metal salt to produce the 5-cholesten-3β,25-diol 3-sulfate metal salt can be achieved such as in (Scheme Ia).

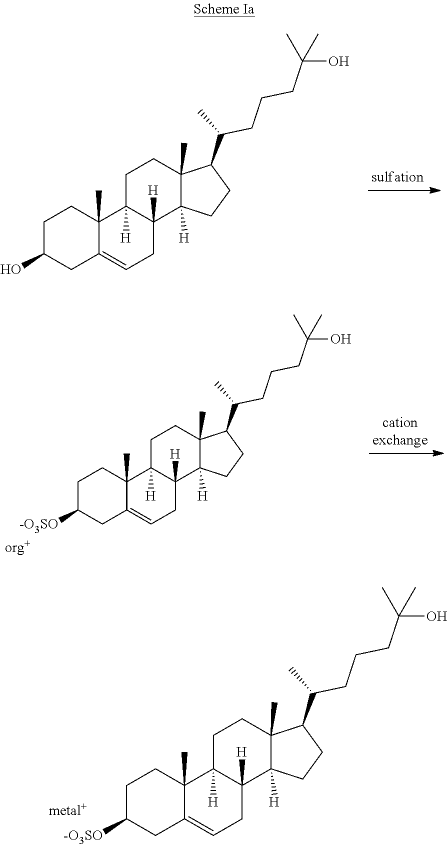

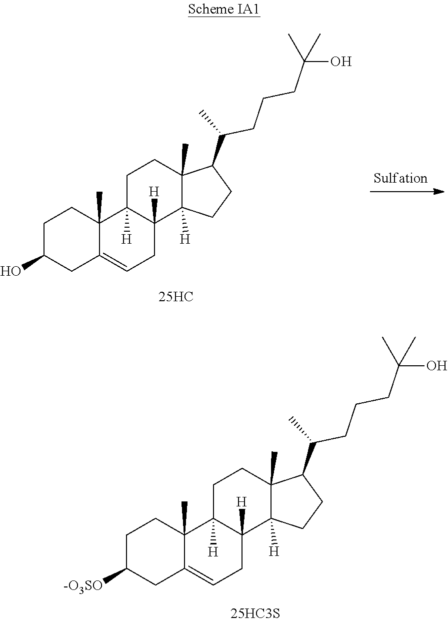

The 25-hydroxy-(3β)-cholest-5-en-3-ol may be sulfated by contacting with a sulfating agent (Scheme IA1). In some cases, the sulfating reagent is chosen from sulfur trioxide complexes, sulfuric acid compounds, sulfonic acid compounds, and sulfonate compounds. In some cases, the sulfating reagent is chosen from sulfur trioxide dimethyl formamide, sulfur trioxide triethylamine, and sulfur trioxide trimethylamine. In some cases, the sulfating reagent includes from sulfuric acid and acetic anhydride and pyridine. In some cases, the sulfating reagent includes sulfur trioxide triethylamine and pyridine. In some cases, the sulfating reagent is chosen from 1) chlorosulfonic acid and pyridine and 2) chlorosulfonic acid and 2,6-lutidine. In some cases, the sulfating reagent is ethyl chlorosulfonate.

The 25-hydroxy-(3β)-cholest-5-en-3-ol may be sulfated at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C., and including from 0° C. to 20° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours and including from 1 hour to 15 hours. The amount of sulfating agent used relative to the 25-hydroxy-(3β)-cholest-5-en-3-ol may vary and may be 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 5 equivalents, 0.5 equivalents to 10 equivalents, 0.5 equivalents to 8 equivalents, 0.5 equivalents to 5 equivalents, 0.9 equivalents to 10 equivalents, 0.9 equivalents to 8 equivalents, 0.9 equivalents to 5 equivalents, 1.3 equivalents to 10 equivalents, 1.3 equivalents to 8 equivalents, 1.3 equivalents to 5 equivalents, 1.5 equivalents to 10 equivalents, 1.5 equivalents to 8 equivalents, 1.5 equivalents to 5 equivalents, 2 equivalents to 10 equivalents, 2 equivalents to 8 equivalents, 2 equivalents to 5 equivalents, or 1 equivalent to 2 equivalents, 1 equivalents to 1.5 equivalents, or 1.1 to 1.2 equivalents, relative to the 25-hydroxy-(3β)-cholest-5-en-3-ol.

In some cases, methods include sulfating the 25-hydroxy-(3β)-cholest-5-en-3-ol in at least one solvent where the 25-hydroxy-(3β)-cholest-5-en-3-sulfate product exhibits low solubility. In some cases, the 25-hydroxy-(3β)-cholest-5-en-3-ol is sulfated in at least one solvent where the 25-hydroxy-(3β)-cholest-5-en-3-sulfate product exhibits a solubility of 100 mmol/L or less, such as 90 mmol/L or less, such as 80 mmol/L or less, such as 70 mmol/L or less, such as 60 mmol/L or less, such as 50 mmol/L or less, such as 40 mmol/L or less, such as 30 mmol/L or less, such as 20 mmol/L or less, such as 10 mmol/L or less, and including sulfating the 25-hydroxy-(3β)-cholest-5-en-3-ol in at least one solvent where the 25-hydroxy-(3β)-cholest-5-en-3-sulfate product exhibits a solubility of 5 mmol/L or less. In some cases, the 25-hydroxy-(3β)-cholest-5-en-3-ol is sulfated in at least one solvent where 25-hydroxy-(3β)-cholest-5-en-3-sulfate product precipitates after formation. In some cases, the at least one solvent is chosen from chloroform, methylene chloride, acetone, acetonitrile, toluene, tetrahydrofuran, and methyltetrahydrofuran.

In some cases, methods include sulfating the 25-hydroxy-(3β)-cholest-5-en-3-ol in a manner sufficient to reduce or eliminate bis-sulfation of the 25-hydroxy-(3β)-cholest-5-en-3-ol. In some instances, the 25-hydroxy-(3β)-cholest-5-en-3-ol is sulfated and a bis-sulfate product (i.e., 5-cholesten-3β-25-diol-disulfate, Structure IA) is formed in an amount that is 10% by weight or less of the reaction product formed by contacting the 25-hydroxy-(3β)-cholest-5-en-3-ol with the sulfating agent, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including where the 25-hydroxy-(3β)-cholest-5-en-3-ol is sulfated and the bis-sulfate product is formed in an amount that is 0.0001% by weight or less, and may range from 10% by weight to 0.001% by weight, such as 10% by weight to 0.1% by weight, 10% by weight to 1% by weight, 10% by weight to 2% by weight, 8% by weight to 0.001% by weight, 8% by weight to 0.1% by weight, 8% by weight to 1% by weight, 8% by weight to 2% by weight, 6% by weight to 0.001% by weight, 6% by weight to 0.1% by weight, 6% by weight to 1% by weight, 6% by weight to 2% by weight, 4% by weight to 0.001% by weight, 4% by weight to 0.1% by weight, 4% by weight to 1% by weight, 4% by weight to 2% by weight, 3% by weight to 0.001% by weight, 3% by weight to 0.1% by weight, 3% by weight to 1% by weight, 2% by weight to 0.001% by weight, 2% by weight to 0.1% by weight, or 2% by weight to 1% by weight.

In some cases, the ratio by weight of the 25-hydroxy-(3β)-cholest-5-en-3-sulfate to the 5-cholesten-3β-25-diol-disulfate formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the 5-cholesten-3β-25-diol-disulfate formed is 109:1 or more, and may range from a ratio by weight of 10:1 to a ratio by weight of 109.1, such as a ratio of weight of 10:1 to a ratio of weight of 10⁶:1, a ratio of weight of 10:1 to a ratio of weight of 103:1, a ratio of weight of 10:1 to a ratio of weight of 100:1, a ratio of weight of 100:1 to a ratio of weight of 109:1, a ratio of weight of 100:1 to a ratio of weight of 10⁶0.1, a ratio of weight of 100:1 to a ratio of weight of 10³:1, a ratio of weight of 250:1 to a ratio of weight of 10⁹:1, a ratio of weight of 250:1 to a ratio of weight of 10⁶:1, a ratio of weight of 250:1 to a ratio of weight of 10³:1, a ratio of weight of 500:1 to a ratio of weight of 10⁹:1, a ratio of weight of 500:1 to a ratio of weight of 10⁶:1, a ratio of weight of 500:1 to a ratio of weight of 10³:1, a ratio of weight of 10³:1 to a ratio of weight of 10⁹:1, a ratio of weight of 10³:1 to a ratio of weight of 10⁶:1, or a ratio of weight of 250:1 to a ratio of weight of 10³:1.

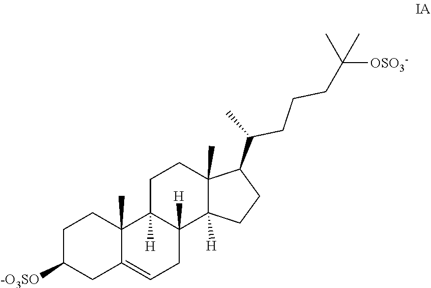

In some cases, the 5-cholesten-3β-25-diol-disulfate formed when sulfating 25-hydroxy-(3β)-cholest-5-en-3-ol remains solubilized in the at least one solvent. In some cases, the 5-cholesten-3β-25-diol-disulfate has high solubility in the at least one solvent. In some instances, the 5-cholesten-3β-25-diol-disulfate exhibits a solubility of 500 mmol/L or more in the at least one solvent, such as 600 mmol/L or more, such as 700 mmol/L or more, such as 800 mmol/L or more, such as 900 mmol/L, or more and including a solubility of 1 mol/L or more in the at least one solvent.

In certain cases, methods further include separating the 25-hydroxy-(3β)-cholest-5-en-3-sulfate product from the bis-sulfate product (i.e., 5-cholesten-3β-25-diol-disulfate). In some cases, the 25-hydroxy-(3β)-cholest-5-en-3-sulfate product is separated from the bis-sulfate product by vacuum filtration. In some cases, the 25-hydroxy-(3β)-cholest-5-en-3-sulfate product is separated from the bis-sulfate product by recrystallization of the 25-hydroxy-(3β)-cholest-5-en-3-sulfate product. In some cases, the 25-hydroxy-(3β)-cholest-5-en-3-sulfate product is separated from the bis-sulfate product by chromatography (e.g., silica column).

In some cases, the 25-hydroxy-(3β)-cholest-5-en-3-ol is sulfated in a reaction mixture having a pH that ranges from 5.0 to 8.0, such as a pH from 5.1 to 7.9, such as a pH from 5.2 to 7.8, such as a pH from 5.3 to 7.7, such as a pH from 5.4 to 7.6, such as a pH from 5.5 to 7.5, such as a pH from 5.6 to 7.4, such as a pH from 5.7 to 7.3, such as a pH from 5.8 to 7.2, such as a pH from 5.9 to 7.1, and including sulfating the 25-hydroxy-(3β)-cholest-5-en-3-ol in a reaction mixture having a pH of from 6.0 to 7.0.

In some cases, 25-hydroxy-(3β)-cholest-5-en-3-ol is sulfated in the presence of a 25-hydroxy-(3β)-cholest-5-en-3-sulfate organic cationic salt. In certain cases, the 25-hydroxy-(3β)-cholest-5-en-3-sulfate organic cationic salt is present as particles (e.g., seed crystals of 25-hydroxy-(3β)-cholest-5-en-3-sulfate organic cationic salt produced in a previous reaction or purified reaction batch). In some cases, sulfating 25-hydroxy-(3β)-cholest-5-en-3-ol in the presence of 25-hydroxy-(3β)-cholest-5-en-3-sulfate organic cationic salt (e.g., as particles) is sufficient to reduce the solubility of 25-hydroxy-(3β)-cholest-5-en-3-sulfate organic cationic salt produced by reaction of the sulfating agent with 25-hydroxy-(3β)-cholest-5-en-3-ol as compared to the solubility when the 25-hydroxy-(3β)-cholest-5-en-3-sulfate organic cationic salt is not present. In certain cases, the solubility of 25-hydroxy-(3′))-cholest-5-en-3-sulfate organic cationic salt produced in the reaction mixture is reduced as compared to the solubility when the added 25-hydroxy-(3β)-cholest-5-en-3-sulfate organic cationic salt is not present by 5% or more, such as by 10% or more, such as by 25% or more, such as by 50% or more, such as by 75% or more, such as by 90% or more and including by reducing the solubility of the produced 25-hydroxy-(3β)-cholest-5-en-3-sulfate organic cationic salt by 99% or more. The size of the particles of 25-hydroxy-(3β)-cholest-5-en-3-sulfate organic cationic salt added to the reaction mixture may vary and may have a dimension (e.g., length, width or diameter) of 0.01 mm or more, such as 0.025 mm or more, such as 0.05 mm or more, such as 0.075 mm or more, such as 0.1 mm or more, such as 0.25 mm or more, such as 0.5 mm or more, such as 0.75 mm or more, such as 1 mm or more, such as 2 mm or more, such as 3 mm or more, such as 4 mm or more and including 5 mm or more. In some cases, the particles of 25-hydroxy-(3β)-cholest-5-en-3-sulfate organic cationic salt are added to the reaction mixture immediately after contacting the sulfating agent with the 25-hydroxy-(3β)-cholest-5-en-3-ol. In some cases, the particles of 25-hydroxy-(3β)-cholest-5-en-3-sulfate organic cationic salt are added to the reaction mixture 1 minute or more after contacting the sulfating agent with the 25-hydroxy-(3β)-cholest-5-en-3-ol, such as 5 minutes or more, such as 10 minutes or more, such as 15 minutes or more, such as 20 minutes or more, such as 30 minutes or more, such as 40 minutes or more, such as 50 minutes or more and including adding the particles of 25-hydroxy-(3β)-cholest-5-en-3-sulfate organic cationic salt to the reaction mixture 60 minutes or more after contacting the sulfating agent with the 25-hydroxy-(3β)-cholest-5-en-3-ol.

In certain cases, the sulfating agent is characterized prior to contacting with the 25-hydroxy-(3β)-cholest-5-en-3-ol. In some cases, characterizing the sulfating agent includes determining the extent of degradation of the sulfating agent prior to contacting with the 25-hydroxy-(3β)-cholest-5-en-3-ol. In certain cases, determining the extent of degradation of the sulfating reagent includes determining the amount of impurity in the sulfating reagent prior to contacting with the 25-hydroxy-(3$p)-cholest-5-en-3-ol.

In some instances, the degradation of the sulfating agent is determined by proton nuclear magnetic resonance spectroscopy (¹H-NMR). Proton NMR spectroscopy of the sulfating agent may be conducted in at least one deuterated solvent. In certain cases, the at least one deuterated solvent is deuterated acetone ((CD₃)₂CO). In certain cases, the at least one deuterated solvent is not deuterated benzene (C₆D₆). In certain cases, the at least one deuterated solvent is not deuterated acetonitrile (CD₃CN). In certain cases, the at least one deuterated solvent is not deuterated chloroform (CD₃Cl).

In some instances, methods for determining the extent of degradation include integrating one or more peaks in the ¹H-NMR spectrum at a chemical shift of from 9.2 ppm to 9.3 ppm and calculating the impurity level of the sulfating agent based on the integrated peaks. In certain instances, methods for determining the extent of degradation include integrating one or more peaks in the ¹H-NMR spectrum at a chemical shift of about 9.25 ppm and calculating the impurity level of the sulfating agent based on the integrated peaks. In some cases, the sulfating agent is contacted with the 25-hydroxy-(3β)-cholest-5-en-3-ol when the impurity level of the sulfating agent is below a predetermined threshold, such as where the impurity level is 25% or less as determined by integrating one or more peaks in the proton NMR spectrum at a chemical shift of from 9.2 ppm to 9.3 ppm, such as 24% or less, such as 23% or less, such as 22% or less, such as 21% or less, such as 20% or less, such as 19% or less, such as 18% or less, such as 17% or less, such as 16% or less, such as 15% or less, such as 14% or less such as 13% or less, such as 12% or less, such as 11% or less, such as 10% or less, such as 9% or less, such as 8% or less, such as 7% or less, such as 6% or less, such as 5% or less, such as 4% or less, such as 3% or less such as 2% or less and including where the impurity level is 1% or less as determined by integrating one or more peaks in the proton NMR spectrum at a chemical shift of from 9.2 ppm to 9.3 ppm. In some cases, the sulfating agent is not contacted with the 25-hydroxy-(3β)-cholest-5-en-3-ol when the impurity level is above a predetermined threshold, such as where the impurity level is 25% or more as determined by integrating one or more peaks in the proton NMR spectrum at a chemical shift of from 9.2 ppm to 9.3 ppm, such as 26% or more, such as 27% or more, such as 28% or more, such as 29% or more, such as 30% or more, such as 31% or more, such as 32% or more, such as 33% or more, such as 34% or more and including where the impurity level is 35% or more as determined by integrating one or more peaks in the proton NMR spectrum at a chemical shift of from 9.2 ppm to 9.3 ppm.

In certain cases, the generated 25-hydroxy-(3β)-cholest-5-en-3-sulfate product includes one or more byproducts. In some cases, the byproduct is 5-cholesten-3β-25-diol-disulfate. In some cases, 5-cholesten-3β-25-diol-disulfate byproduct is present in the composition produced by sulfation of 25-hydroxy-(3β)-cholest-5-en-3-ol in an amount relative to the 25-hydroxy-(3β)-cholest-5-en-3-sulfate of 10% by weight or less, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including where 5-cholesten-3β-25-diol-disulfate byproduct is present in the composition produced by sulfation of 25-hydroxy-(3β)-cholest-5-en-3-ol in an amount of 0.001% by weight or less, and may range from 0.1% by weight to 50% by weight, such as 0.5% by weight to 20% by weight or 1% by weight to 12% by weight. In some cases, the ratio by weight of the 25-hydroxy-(3β)-cholest-5-en-3-sulfate to the 5-cholesten-3β-25-diol-disulfate byproduct formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 107:1 or more, such as 10⁸:1 or more, and including where the ratio by weight of the 25-hydroxy-(3β)-cholest-5-en-3-sulfate to the 5-cholesten-3β-25-diol-disulfate formed is 10⁹:1 or more. In some cases, the ratio by weight of the 25-hydroxy-(3β)-cholest-5-en-3-sulfate and the 5-cholesten-3β-25-diol-disulfate formed ranges from 10:1 to 10⁹:1, such as from 100:1 to 10⁸0.1, such as from 1000:1 to 107:1, and including from 10000:1 to 10⁶:1.

Aspects of the present disclosure also include compositions having 25-hydroxy-(3β)-cholest-5-en-3-sulfate and 5-cholesten-3β-25-diol-disulfate that is present in the composition in an amount relative to the 25-hydroxy-(3β)-cholest-5-en-3-sulfate of 10% by weight or less, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including 0.001% by weight or less, and may range from 10% by weight to 0.001% by weight, such as 10% by weight to 0.1% by weight, 10% by weight to 1% by weight, 10% by weight to 2% by weight, 8% by weight to 0.001% by weight, 8% by weight to 0.1% by weight, 8% by weight to 1% by weight, 8% by weight to 2% by weight, 6% by weight to 0.001% by weight, 6% by weight to 0.1% by weight, 6% by weight to 1% by weight, 6% by weight to 2% by weight, 4% by weight to 0.001% by weight, 4% by weight to 0.1% by weight, 4% by weight to 1% by weight, 4% by weight to 2% by weight, 3% by weight to 0.001% by weight, 3% by weight to 0.1% by weight, 3% by weight to 1% by weight, 2% by weight to 0.001% by weight, 2% by weight to 0.1% by weight, or 2% by weight to 1% by weight.

In some cases, compositions include a ratio by weight of the 25-hydroxy-(3β)-cholest-5-en-3-sulfate and the 5-cholesten-3β-25-diol-disulfate of 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 107:1 or more, such as 108:1 or more, and including where the ratio by weight of the 25-hydroxy-(33)-cholest-5-en-3-sulfate to the 5-cholesten-3β-25-diol-disulfate in the composition is 10⁹:1 or more. In some cases, compositions include a ratio by weight of the 25-hydroxy-(3β)-cholest-5-en-3-sulfate and the 5-cholesten-3β-25-diol-disulfate that ranges from 10:1 to 10⁹:1, such as from 100:1 to 10⁸:1, such as from 1000:1 to 10⁷:1, and including from 10000:1 to 10⁶:1.

In some cases, the byproduct is sulfated desmosterol (Structure IB).

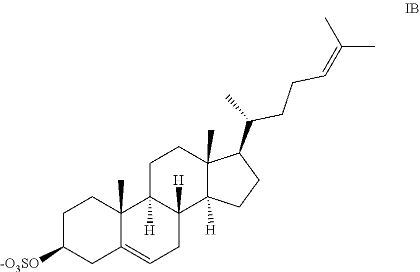

In some cases, sulfated desmosterol ([(3S,8S,9S,10R,13R,14S,17R)-17-[(1R)-1,5-dimethylhex-4-enyl]-10,13-dimethyl-2,3,4,7,8,9,11,12,14,15,16,17-dodecahydro-1H-cyclopenta[a]phenanthren-3-yl] sulfate) is present in the composition produced by sulfation of 25-hydroxy-(3β)-cholest-5-en-3-ol in an amount relative to the 25-hydroxy-(3β)-cholest-5-en-3-sulfate of 10% by weight or less, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including where sulfated desmosterol is present in the composition produced by sulfation of 25-hydroxy-(3β)-cholest-5-en-3-ol in an amount relative to the 25-hydroxy-(3β)-cholest-5-en-3-sulfate of 0.001% by weight or less, and may range from 0.1% by weight to 10% by weight, such as 0.2% by weight to 5% by weight or 0.3% by weight to 3% by weight. In some cases, the ratio by weight of the 25-hydroxy-(33)-cholest-5-en-3-sulfate to the sulfated desmosterol formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(33)-cholest-5-en-3-sulfate to the sulfated desmosterol formed is 10⁹:1 or more. In some cases, the ratio by weight of the 25-hydroxy-(3β)-cholest-5-en-3-sulfate and the sulfated desmosterol formed ranges from 10:1 to 10⁹:1, such as from 100:1 to 10⁸:1, such as from 1000:1 to 10⁷:1 and including from 10000:1 to 10⁶:1.

Aspects of the present disclosure also include compositions having 25-hydroxy-(3β)-cholest-5-en-3-sulfate and sulfated desmosterol that is present in the composition in an amount relative to the 25-hydroxy-(3β)-cholest-5-en-3-sulfate of 10% by weight or less, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including 0.001% w/w or less relative to the 25-hydroxy-(3β)-cholest-5-en-3-sulfate, and may range from 10% by weight to 0.001% by weight, such as 10% by weight to 0.1% by weight, 10% by weight to 1% by weight, 10% by weight to 2% by weight, 8% by weight to 0.001% by weight, 8% by weight to 0.1% by weight, 8% by weight to 1% by weight, 8% by weight to 2% by weight, 6% by weight to 0.001% by weight, 6% by weight to 0.1% by weight, 6% by weight to 1% by weight, 6% by weight to 2% by weight, 4% by weight to 0.001% by weight, 4% by weight to 0.1% by weight, 4% by weight to 1% by weight, 4% by weight to 2% by weight, 3% by weight to 0.001% by weight, 3% by weight to 0.1% by weight, 3% by weight to 1% by weight, 2% by weight to 0.001% by weight, 2% by weight to 0.1% by weight, or 2% by weight to 1% by weight.

In some cases, compositions include a ratio by weight of the 25-hydroxy-(3β)-cholest-5-en-3-sulfate and the sulfated desmosterol of 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 107:1 or more, such as 10⁸:1 or more, and including where the ratio by weight of the 25-hydroxy-(3β)-cholest-5-en-3-sulfate to the sulfated desmosterol in the composition is 10⁹:1 or more. In some cases, compositions include a ratio by weight of the 25-hydroxy-(3β)-cholest-5-en-3-sulfate and the sulfated desmosterol that ranges from 10:1 to 10⁹:1, such as from 100:1 to 10⁸:1, such as from 1000:1 to 10′:1 and including from 10000:1 to 10⁶:1.

In some cases, the byproduct of sulfating the 25-hydroxy-(3β)-cholest-5-en-3-ol that is present in the 25-hydroxy-(3β)-cholest-5-en-3-sulfate composition is a thermal degradation product. In some cases, the byproduct is identified by relative retention time when the components of the 25-hydroxy-(3β)-cholest-5-en-3-sulfate composition are separated by liquid chromatography (e.g., HPLC). In certain cases, the byproduct is sulfated desmosterol, a compound having a retention time of about 18.3 minutes when the components of the 25-hydroxy-(3β)-cholest-5-en-3-sulfate composition are separated by HPLC operating at about 45° C. with a C₈ stationary phase and separates the components of the composition with a first mobile phase comprising a buffer (e.g., an aqueous buffer of sodium phosphate) and a second mobile phase comprising one or more organic solvents (see e.g., Tables 3 and 4 below). In some cases, the first mobile phase is an aqueous buffer. In certain cases, the first mobile phase includes sodium phosphate. In some cases, the second mobile phase is chosen from one or more of methoxypropyl acetate, acetonitrile and methanol. In some cases, the flow rate of the first mobile phase is about 1.0 mL/minute. In some cases, the flow rate of the second mobile phase is about 1.0 mL/minute or more. In some cases, 25-hydroxy-(3β)-cholest-5-en-3-sulfate has a retention time of about 7.7 minutes under the same HPLC conditions. In some cases, the byproduct is a compound having a retention time of about 37.7 minutes when the components of the 25-hydroxy-(3β)-cholest-5-en-3-sulfate composition are separated by HPLC operating at about 45° C. with a C₈ stationary phase and separates the components of the composition with a first mobile phase comprising a buffer (e.g., an aqueous buffer of sodium phosphate) and a second mobile phase comprising one or more organic solvents (see e.g., Tables 3 and 4 below). While not wishing to be bound by theory, it is believed that the compound having a retention time of about 37.7 minutes is desmosterol. In some cases, the first mobile phase is an aqueous buffer. In certain cases, the first mobile phase includes sodium phosphate. In some cases, the second mobile phase is chosen from one or more of methoxypropyl acetate, acetonitrile and methanol. In some cases, the flow rate of the first mobile phase is about 1.0 mL/minute. In some cases, the flow rate of the second mobile phase is about 1.0 mL/minute or more. In some cases, and 25-hydroxy-(3β)-cholest-5-en-3-sulfate has a retention time of about 7.7 minutes under the same HPLC conditions such that sulfated desmosterol has a relative retention time of about 2.4 (=18.3/7.7) and the compound believed to be desmosterol has a relative retention time of about 4.9 (=37.7/7.7).

Aspects of the present disclosure also include compositions having 25-hydroxy-(3β)-cholest-5-en-3-sulfate and one or more byproducts of sulfating the 25-hydroxy-(3β)-cholest-5-en-3-ol. In some cases, the one or more byproducts are present in the composition in an amount relative to the 25-hydroxy-(3β)-cholest-5-en-3-sulfate of 10% by weight or less, such as 9% by weight or less, such as 8% by weight or less, such as 7% by weight or less, such as 6% by weight or less, such as 5% by weight or less, such as 4% by weight or less, such as 3% by weight or less, such as 2% by weight or less, such as 1% by weight or less, such as 0.5% by weight or less, such as 0.1% by weight or less, such as 0.01% by weight or less, such as 0.001% by weight or less, and including 0.001% by weight or less, and may range from 0.1% by weight to 5% by weight, such as 0.2% by weight to 10% by weight or 0.3% by weight to 15% by weight. In some cases, compositions include 25-hydroxy-(3β)-cholest-5-en-3-sulfate and the one or more byproducts in an amount relative to the 25-hydroxy-(3β)-cholest-5-en-3-sulfate that ranges from 0.0001% by weight to 10% by weight, such as from 0.005% by weight to 9.5% by weight, such as from 0.001% to 9.0% by weight, such as from 0.05% by weight to 8.5% by weight, such as from 0.1% by weight to 8.0% by weight, such as from 0.5% by weight to 7.5% by weight, such as from 1% by weight to 7% by weight, such as from 1.5% by weight to 6.5% by weight, and including from 2% by weight to 6% by weight.

In some cases, the ratio by weight of the 25-hydroxy-(3β)-cholest-5-en-3-sulfate to the one or more byproducts formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 107:1 or more, such as 10⁸:1 or more, and including where the ratio by weight of the 25-hydroxy-(3β)-cholest-5-en-3-sulfate to the one or more byproducts formed is 10⁹:1 or more. In some cases, the ratio by weight of the 25-hydroxy-(3β)-cholest-5-en-3-sulfate and the one or more byproducts formed ranges from 10:1 to 10⁹:1, such as from 100:1 to 10⁸:1, such as from 1000:1 to 107:1, and including from 10000:1 to 10⁶:1.

In some cases, the 25-hydroxy-(3β)-cholest-5-en-3-sulfate organic cationic salt is a 25-hydroxy-(3β)-cholest-5-en-3-sulfate pyridinium salt (Scheme IA2).

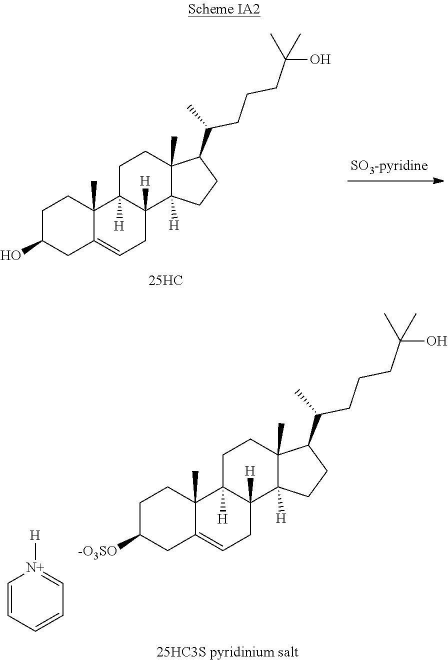

In certain cases, the sulfating agent is contacted with an anhydride prior to contacting with the 25-hydroxy-(3β)-cholest-5-en-3-ol. In some cases, the anhydride is chosen from acetic anhydride, trifluoroacetic anhydride and triflic anhydride. The amount of anhydride relative to the 25-hydroxy-(3β)-cholest-5-en-3-ol may vary and may be 0.001 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 5 equivalents, 0.5 equivalents to 10 equivalents, 0.5 equivalents to 8 equivalents, 0.5 equivalents to 5 equivalents, 0.9 equivalents to 10 equivalents, 0.9 equivalents to 8 equivalents, 0.9 equivalents to 5 equivalents, 1.3 equivalents to 10 equivalents, 1.3 equivalents to 8 equivalents, 1.3 equivalents to 5 equivalents, 1.5 equivalents to 10 equivalents, 1.5 equivalents to 8 equivalents, 1.5 equivalents to 5 equivalents, 2 equivalents to 10 equivalents, 2 equivalents to 8 equivalents, 2 equivalents to 5 equivalents, 0.1 equivalent to 1.5 equivalents, 0.5 equivalents to 1.1 equivalents, or 0.1 equivalent to 1 equivalent relative to the 25-hydroxy-(3β)-cholest-5-en-3-ol.

In some cases, methods include quenching (i.e., deactivating) unreacted sulfating agent after producing the 25-hydroxy-(3β)-cholest-5-en-3-sulfate organic cationic salt. In some cases, quenching the sulfating agent includes adding water to the reaction mixture. The amount of water added to the reaction mixture relative to the amount of sulfating agent contacted with the 25-hydroxy-(3f3)-cholest-5-en-3-ol may vary and may be 1 equivalent or more, such as 2 equivalents or more, such as 3 equivalents or more, such as 4 equivalents or more, such as 5 equivalents or more, such as 6 equivalents or more, such as 7 equivalents or more, such as 8 equivalents or more, such as 9 equivalents or more, such as 10 equivalents or more, such as 15 equivalents or more, such as 20 equivalents or more and including 25 equivalents or more.

In certain cases, quenching the reactivity of unreacted sulfating agent includes adding water to the reaction mixture followed by the addition of at least one base. In some cases, the at least one base is a trialkylamine, such as trimethylamine or triethylamine. In some cases, the at least one base is 2,6-lutidine. In certain cases, the at least one base is pyridine. The pyridine may be added to the reaction mixture 1 minute or more after adding the water, such as 5 minutes or more, such as 10 minutes or more, such as 15 minutes or more, such as 30 minutes or more, such as 45 minutes or more, such as 60 minutes or more, such as 90 minutes or more, such as 120 minutes or more, such as 150 minutes or more, such as 180 minutes or more, such as 210 minutes or more and including 240 minutes or more after adding the water to the reaction mixture. In certain cases, pyridine is added to the reaction mixture 60 minutes after adding the water. The amount of pyridine added to the reaction mixture relative to the amount of sulfating agent may vary and may be 0.001 equivalents or more, such as 0.005 equivalents or more, such as 0.01 equivalents or more, such as 0.05 equivalents or more, such as 0.1 equivalents or more, such as 0.5 equivalents or more, such as 1 equivalent or more, such as 2 equivalents or more, such as 3 equivalents or more, such as 4 equivalents or more, such as 5 equivalents or more, such as 6 equivalents or more and including 10 equivalents or more.

In some cases, the unreacted sulfating agent in the reaction mixture is quenched under slow agitation. In certain cases, quenching the unreacted sulfating agent under slow agitation includes stirring the reaction mixture in a manner sufficient to maintain agglomerates of the unreacted sulfating agent in the reaction mixture. In some cases, slow agitation of the reaction mixture is sufficient such that agglomerates of unreacted sulfating agent reduce in size during quenching by 10% or less, such as by 9% or less, such as by 8% or less, such as by 7% or less, such as by 6% or less, such as by 5% or less, such as by 4% or less, such as by 3% or less, such as by 2% or less, such as by 1% or less and including where the reaction mixture is slowly agitated such that agglomerates of unreacted sulfating agent reduce in size during quenching by 0.1% or less. In certain cases, slow agitation of the reaction mixture is sufficient such that agglomerates of unreacted sulfating agent remain at the bottom of the reaction flask during quenching. In certain cases, slow agitation of the reaction mixture is sufficient such that little to no agglomerates of unreacted sulfating agent is present in the stirring vortex of the agitated reaction mixture.

In some cases, methods include purifying the 25-hydroxy-(3$0)-cholest-5-en-3-sulfate organic cationic salt prior to contacting the 25-hydroxy-(3β)-cholest-5-en-3-sulfate organic cationic salt with the at least one metal salt. In some cases, the purified 25-hydroxy-(3β)-cholest-5-en-3-sulfate organic cationic salt has a purity of 97% or greater, such as a purity of 98% or greater, such as a purity of 99% or greater, such as purity of 99.5% or greater, such as purity of 99.7% or greater, such as a purity of 99.9% or greater and including a purity of 99.99% or greater. In certain cases, the purified 25-hydroxy-(3β)-cholest-5-en-3-sulfate organic cationic salt has one or more by-products of sulfation (e.g., by-products from sulfating the 25-hydroxy-(3β)-cholest-5-en-3-ol) where the one or more by-products is present in an amount of 5% w/w or less relative to the 25-hydroxy-(3β)-cholest-5-en-3-sulfate organic cationic salt, such as 4% w/w or less, such as 3% w/w or less, such as 2% w/w or less, such as 1% w/w or less, such as in an amount of 0.9% w/w or less, such as 0.8% w/w or less, such as 0.7% w/w or less, such as 0.6% w/w or less, such as 0.5% w/w or less, such as 0.4% w/w or less, such as 0.3% w/w or less, such as 0.2% w/w or less, such as 0.1% w/w or less, such as 0.05% w/w or less, such as 0.01% w/w or less and including being present in an amount of 0.001% w/w or less relative to the 25-hydroxy-(3β)-cholest-5-en-3-sulfate organic cationic salt. In some cases, the bis-sulfated product (i.e., 5-cholesten-3β-25-diol-disulfate) is present in the purified 25-hydroxy-(3β)-cholest-5-en-3-sulfate organic cationic salt composition in an amount of 1% w/w or less relative to the 25-hydroxy-(3β)-cholest-5-en-3-sulfate organic cationic salt, such as in an amount of 0.9% w/w or less, such as 0.8% w/w or less, such as 0.7% w/w or less, such as 0.6% w/w or less, such as 0.5% w/w or less, such as 0.4% w/w or less, such as 0.3% w/w or less, such as 0.2% w/w or less, such as 0.1% w/w or less, such as 0.05% w/w or less, such as 0.01% w/w or less and including being present in an amount of 0.001% w/w or less relative to the 25-hydroxy-(3β)-cholest-5-en-3-sulfate organic cationic salt.

In some cases, the 25-hydroxy-(3β)-cholest-5-en-3-sulfate organic cationic salt is purified by liquid chromatography. In some cases, purifying the 25-hydroxy-(3β)-cholest-5-en-3-sulfate organic cationic salt includes liquid chromatography using a silica gel stationary phase (e.g., a silica gel plug column, ≥5 mass equivalents). In some cases, the 25-hydroxy-(3β)-cholest-5-en-3-sulfate organic cationic salt is purified using the silica gel stationary phase and a mobile phase that includes pyridine. In certain cases, the mobile phase includes methylene chloride, methanol, and pyridine. In certain cases, the mobile phase includes a mixture of methylene chloride-methanol (85:15) and pyridine (1%).

In some cases, one or more fractions collected from the stationary phase may be combined. In some cases, the combined fractions may be concentrated. In certain cases, the combined fractions are concentrated by distillation. In certain cases, the combined fractions are concentrated under vacuum. In certain cases, the combined fractions are concentrated by distillation under vacuum.

In some cases, the combined fractions are contacted with one or more particles of the 25-hydroxy-(3β)-cholest-5-en-3-sulfate organic cationic salt (e.g., particles from a previously purified sample of the 25-hydroxy-(3β)-cholest-5-en-3-sulfate organic cationic salt). In some cases, contacting the particles of the 25-hydroxy-(3$P)-cholest-5-en-3-sulfate organic cationic salt with the combined fractions is sufficient to precipitate 25-hydroxy-(3β)-cholest-5-en-3-sulfate organic cationic salt in the combined fractions. In some cases, contacting particles of the 25-hydroxy-(3β)-cholest-5-en-3-sulfate organic cationic salt with the combined fractions includes adding the particles during distillation of the combined fractions. In some cases, the particles of 25-hydroxy-(3β)-cholest-5-en-3-sulfate organic cationic salt are added to the combined fractions before distilling the combined fractions. In some cases, the particles of 25-hydroxy-(3β)-cholest-5-en-3-sulfate organic cationic salt are added to the combined fractions while distilling the combined fractions, such as 1 minute or more after beginning the distillation, such as 5 minutes or more, such as 10 minutes or more, such as 15 minutes or more, such as 20 minutes or more, such as 30 minutes or more, such as 40 minutes or more, such as 50 minutes or more and including adding the particles of 25-hydroxy-(3β)-cholest-5-en-3-sulfate organic cationic salt to the combined fractions 60 minutes or more after beginning the distillation of the combined fractions. In certain cases, the combined fractions are distilled under constant pressure, such as where the pressure changes by 10% or less, such as by 9% or less, such as by 8% or less, such as by 7% or less, such as by 6% or less, such as by 5% or less, such as by 4% or less, such as by 3% or less, such as by 2% or less, such as by 1% or less and including by 0.1% or less. In some cases, the pressure during distillation changes by 10 inHg or less, such as by 9 inHg or less, such as by 8 inHg or less, such as by 7 inHg or less, such as by 6 inHg or less, such as by 5 inHg or less, such as by 4 inHg or less, such as by 3 inHg or less, such as by 2 inHg or less, such as by 1 inHg or less, such as by 0.5 inHg or less, such as by 0.1 inHg or less, such as by 0.05 inHg or less and including by 0.01 inHg or less. In some cases, the combined fractions are distilled under a reduced pressure wherein the pressure is maintained between 15 inHg to 30 inHg, such as from 17.5 inHg to 27.5 inHg, such as from 20 inHg to 25 inHg, such as from 21 inHg and 24 inHg and including maintained at a pressure of from 22 inHg to 23 inHg.

In some cases, the combined fractions are concentrated under vacuum and the concentrated combined fractions are contacted with a composition containing particles of the 25-hydroxy-(3β)-cholest-5-en-3-sulfate organic cationic salt. In certain cases, the concentrated combined fractions are contacted with a composition containing particles of the 25-hydroxy-(3β)-cholest-5-en-3-sulfate organic cationic salt and at least one solvent. In certain cases, the at least one solvent is chosen from tetrahydrofurans, such as 2-methyltetrahydrofuran. The concentrated combined fractions may be contacted with the composition containing the particles of the 25-hydroxy-(3β)-cholest-5-en-3-sulfate organic cationic salt over a duration of 0.001 minutes or more, such as over 0.005 minutes or more, such as over 0.01 minutes or more, such as over 0.05 minutes or more, such as over 0.1 minutes or more, such as over 0.5 minutes or more, such as over 1 minute or more, such as over 2 minutes or more, such as over 3 minutes or more, such as over 4 minutes or more, such as over 5 minutes or more, such as over 10 minutes or more, such as over 15 minutes or more, such as over 30 minutes or more, such as over 45 minutes or more and including over 60 minutes or more. In certain cases, the combined fractions are added dropwise to a composition containing 25-hydroxy-(3β)-cholest-5-en-3-sulfate organic cationic salt in 2-methyltetrahydrofuran.

In some cases, the 25-hydroxy-(3β)-cholest-5-en-3-sulfate organic cationic salt is contacted with a metal salt to produce the 25-hydroxy-(3β)-cholest-5-en-3-sulfate metal salt (Scheme IB1).

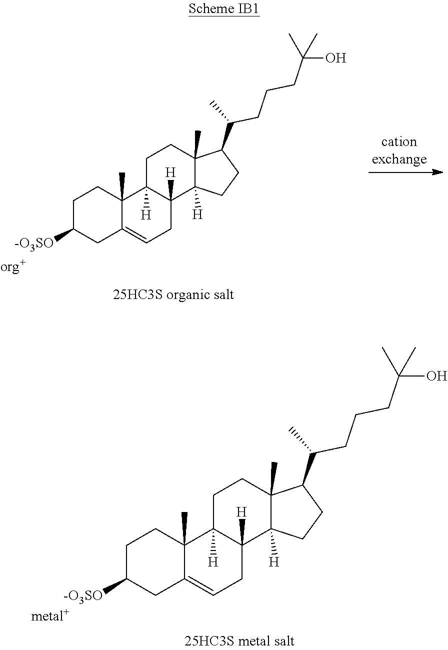

In some cases, methods to produce the 25-hydroxy-(3β)-cholest-5-en-3-sulfate metal salt includes contacting the 25-hydroxy-(3β)-cholest-5-en-3-sulfate organic cationic salt with at least one sodium salt. In some cases, the at least one sodium salt is chosen from sodium acetate, sodium iodide, sodium chloride, sodium hydroxide and sodium methoxide. The 25-hydroxy-(3β)-cholest-5-en-3-sulfate organic cationic salt may be contacted with the metal salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C., such as from 0° C. to 45° C., such as from 5° C. to 40° C., and including from 10° C. to 35° C.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours. The amount of metal salt used relative to the 25-hydroxy-(3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, or 2 equivalents to 3 equivalents, 1 equivalent to 100 equivalents, 1 equivalent to 5 equivalents, 1 equivalent to 2 equivalents.

In some cases, methods include contacting the 25-hydroxy-(3)-cholest-5-en-3-sulfate pyridinium salt with sodium iodide to produce a 25-hydroxy-(3β)-cholest-5-en-3-sulfate sodium salt (Scheme IB2).

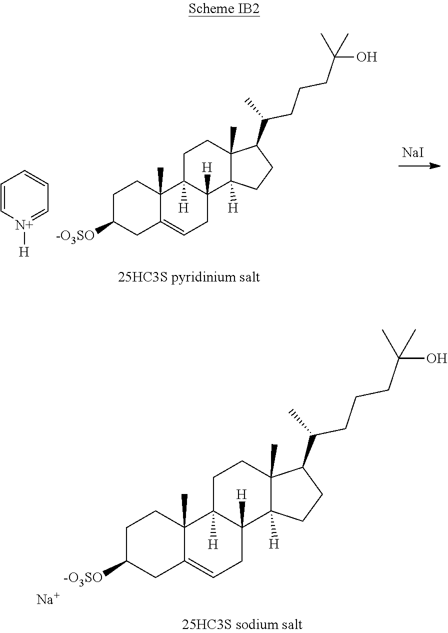

In some cases, methods for preparing 25-hydroxy-3β-cholesten-5-en-3-sulfate include contacting 25-hydroxy-(3β)-cholest-5-en-3-ol with a sulfur trioxide-pyridine complex to produce a 25-hydroxy-(3β)-cholest-5-en-3-sulfate pyridinium salt; and contacting the 25-hydroxy-(3β)-cholest-5-en-3-sulfate pyridinium salt with a sodium salt to produce the 5-cholesten-3β,25-diol 3-sulfate sodium salt (Scheme Ib).

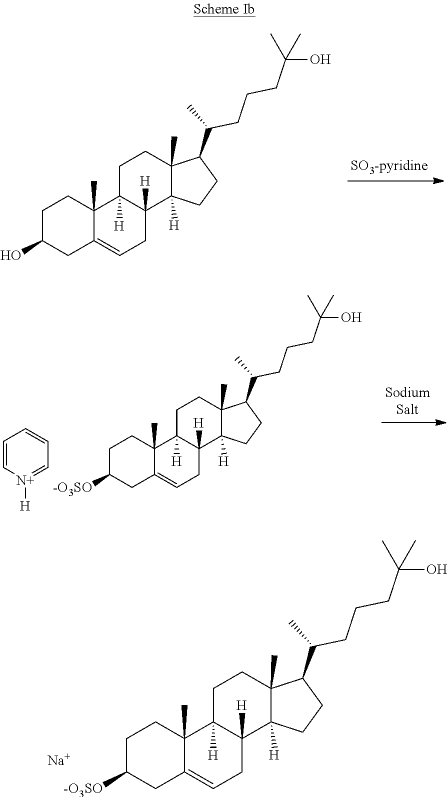

In some cases, methods for preparing 25-hydroxy-3β-cholesten-5-en-3-sulfate include contacting (3β)-cholest-5-en-3-ol with a sulfating agent to produce a first (3β)-cholest-5-en-3-sulfate organic cationic salt; contacting the first (3β)-cholest-5-en-3-sulfate organic cationic salt with an organic base to produce a second (3β)-cholest-5-en-3-sulfate organic cationic salt; oxidizing the second (3β)-cholest-5-en-3-sulfate organic cationic salt in the presence of at least one surfactant to produce a 25-hydroxy-(3β)-cholest-(5,6-epoxy)-3-sulfate organic cationic salt; generating a 25-hydroxy-(3β)-cholest-5-en-3-sulfate organic cationic salt from the 25-hydroxy-(3β)-cholest-(5,6-epoxy)-3-sulfate organic cationic salt by deoxygenation; and contacting the 25-hydroxy-(3β)-cholest-5-en-3-sulfate organic cationic salt with at least one metal salt to produce the 5-cholesten-30,25-diol 3-sulfate metal salt (Scheme IIa).

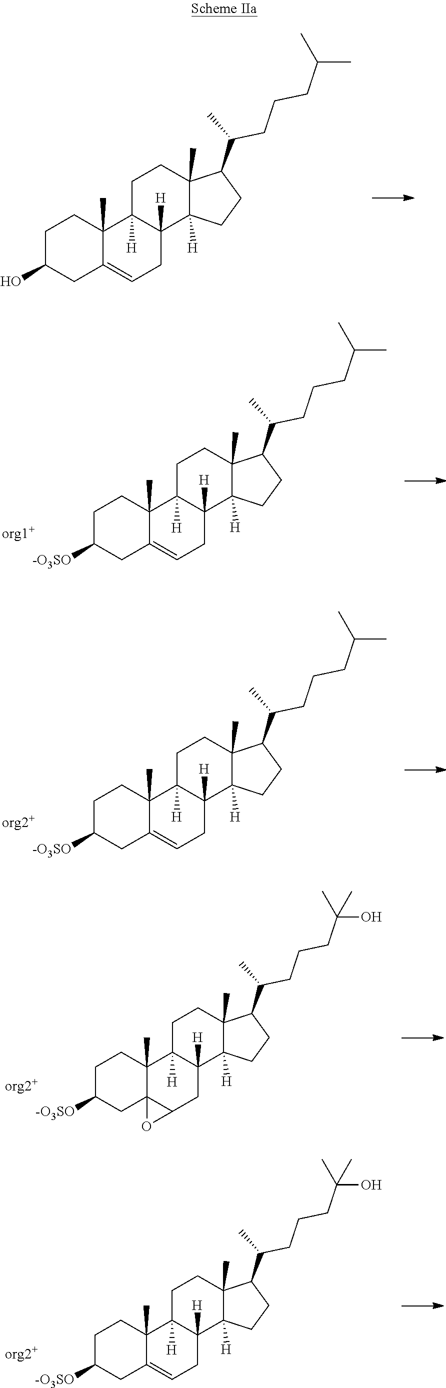

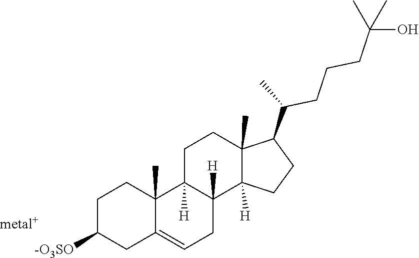

In some cases, cholesterol is sulfated with a sulfating agent (Scheme IIA1). In some cases, the sulfating agent is chosen from sulfur trioxide complexes, sulfuric acid compounds, sulfonic acid compounds, and sulfonate compounds. In some cases, the sulfating agent is a sulfur trioxide-pyridine complex. In some cases, the sulfating agent is chosen from sulfur trioxide dimethyl formamide, sulfur trioxide triethylamine, and sulfur trioxide trimethylamine. In some cases, the sulfating agent is sulfuric acid and acetic anhydride and pyridine. In some cases, the sulfating agent is chosen from chlorosulfonic acid and pyridine. In some cases, the sulfating agent is chosen from chlorosulfonic acid and 2,6-lutidine. In some cases, the sulfating agent is chosen from ethyl chlorosulfonate.

Cholesterol may be sulfated at a temperature that ranges from 0° C. to 100° C., such as from 5° C. to 95° C., such as from 10° C. to 90° C., such as from 15° C. to 85° C., such as from 20° C. to 80° C., such as from 25° C. to 75° C., and including from 30° C. to 70° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, and including from 0.7 hours to 18 hours. The amount of sulfating agent used relative to cholesterol may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 30 equivalents, 1 equivalent to 5 equivalents, or 1 equivalent to 2 equivalents.

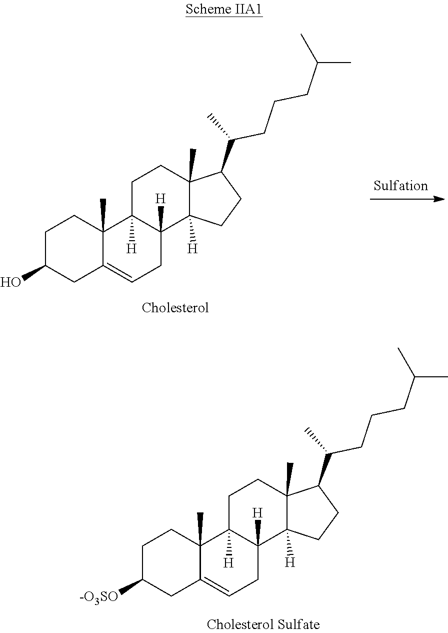

In some cases, the first (3β)-cholest-5-en-3-sulfate organic cationic salt is a (3β)-cholest-5-en-3-sulfate pyridinium salt (Scheme IIA2).

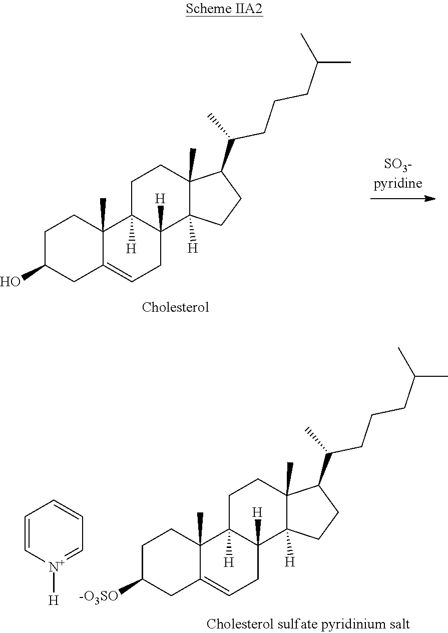

In some cases, the first (3β)-cholest-5-en-3-sulfate organic cationic salt (Structure IIA) is contacted with an organic base to produce a second (3β)-cholest-5-en-3-sulfate organic cationic salt (Structure IIB) (Scheme IIB1).

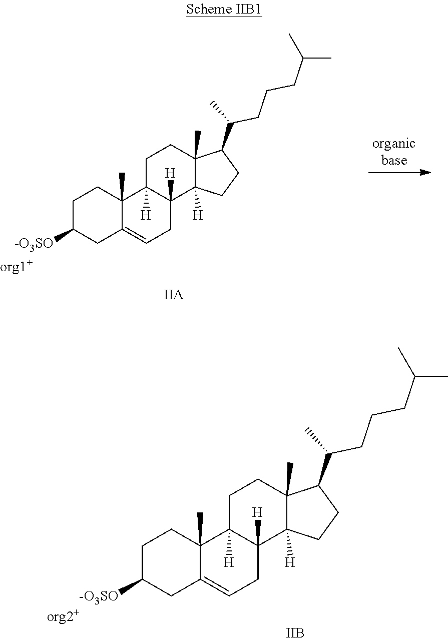

In some cases, the organic base contacted with the first (3β)-cholest-5-en-3-sulfate organic cationic salt is chosen from a hydroxide base. In some cases, the hydroxide base is chosen from tetraethylammonium hydroxide, tetrabutylammonium hydroxide, tetrapropylammonium hydroxide and tetramethylammonium hydroxide. In some cases, the second (3β)-cholest-5-en-3-sulfate organic cationic salt is chosen from a tetraethylammonium cationic salt, a tetrabutylammonium cationic salt, a tetrapropylammonium cationic salt and a tetramethylammonium cationic salt. In some cases, the organic base is contacted with the first (3β)-cholest-5-en-3-sulfate organic cationic salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C. and including from 0° C. to 15° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hour to 15 hours. The amount of the organic base used relative to the first (3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 10 equivalents, 1 equivalent to 5 equivalents, or 1 equivalent to 2 equivalents.

In certain cases, methods include contacting the first (3β)-cholest-5-en-3-sulfate organic cationic salt with tetrabutylammonium hydroxide to generate a (3β)-cholest-5-en-3-sulfate tetrabutylammonium cationic salt (Structure IIB1) (Scheme IIB2).

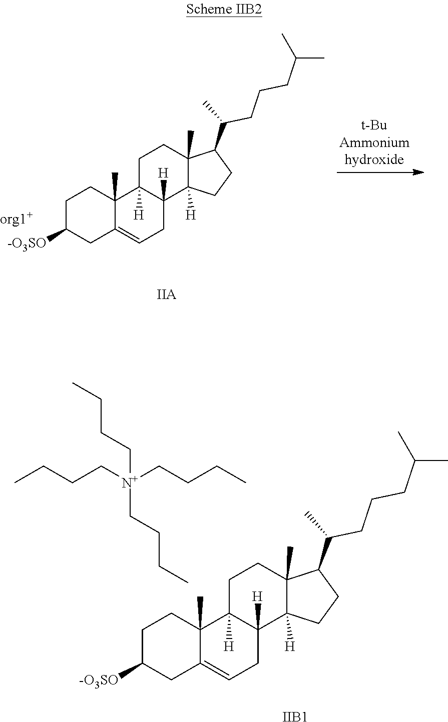

In some cases, the second (3β)-cholest-5-en-3-sulfate organic cationic salt is oxidized to produce a 25-hydroxy-(3$P)-cholest-(5,6-epoxy)-3-sulfate organic cationic salt (Structure IIC) (Scheme IIC1).

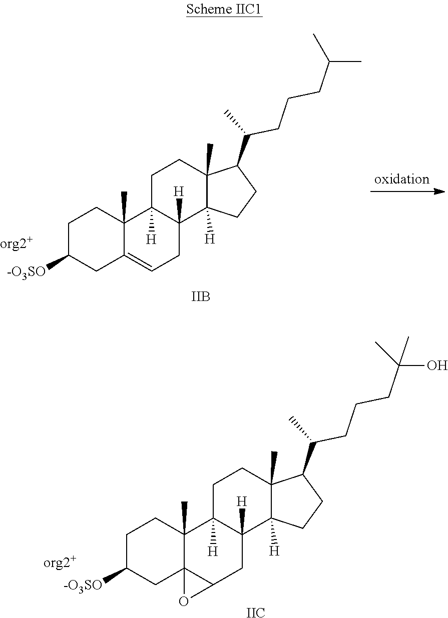

In some cases, oxidizing the second (3β)-cholest-5-en-3-sulfate organic cationic salt includes contacting the second (3β)-cholest-5-en-3-sulfate organic cationic salt with a composition having an oxidizing agent and at least one surfactant.

In some cases, the at least one surfactant is chosen from non-ionic surfactants, anionic surfactants, cationic surfactants and zwitterionic surfactants. Non-ionic surfactants may be chosen from polyoxyethylene glycol ethers (e.g., polyoxyethylene glycol octylphenol ether), polyoxyethylene glycol sorbitan alkyl esters, sorbitan alkyl esters, block copolymers of polyethylene glycol and polypropylene glycol, among other non-ionic surfactants. Anionic surfactants may be chosen from surfactants having an anionic functional head group, such as a sulfonate, phosphate, sulfate or carboxylate head group-containing surfactant. For example, anionic surfactants may be chosen from alkyl sulfates such as ammonium lauryl sulfate, dioctyl sodium sulfosuccinate, perfluorooctanesulfonate, perfluorononanoate, perfluorooctanoate, a linear alkylbenzene sulfonate, an alkyl-aryl ether phosphate, sodium lauryl ether sulfate, lignosulfonate or sodium stearate, among other anionic surfactants. Cationic surfactants may be chosen from surfactants having a cationic functional head group, such as a pyridinium or a quarternary ammonium head group. For example, cationic surfactants may be chosen from cetyltrimethylammonium hydrogen sulfate, tetrabutylammonium hydrogen sulfate, cetyltrimethylammonium bromide, tetrabutylammonium bromide, tetrabutylammonium iodide, tetrabutylphosphonium bromide, tetraoctylammonium bromide, tetraoctylammonium iodide, benzyltriethylammonium chloride, benzyltriethylammonium bromide, benzylcetyldimethylammonium chloride or benzylcetyldimethylammonium bromide. Zwitterionic surfactants include both cationic and anionic centers, such as a sultaine (e.g., 3-[(3-cholamidopropyl)dimethylammonio]-1-propanesulfonate) or a betaine (e.g., cocamidopropyl betaine). In certain cases, the at least one surfactant is an Extran laboratory soap, La Parisienne soap or DL-α-tocopherol methoxypolyethylene glycol succinate (e.g., TPGS-750-M-2).

The amount of surfactant used relative to the second (3β)-cholest-5-en-3-sulfate organic cationic salt may vary, where in some instances, 0.0001 equivalents or more of the surfactant is used, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more of the surfactant, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 0.1 equivalent to 5 equivalents, 0.15 equivalents to 1 equivalent, or 0.2 equivalents to 0.3 equivalents.

In some cases, oxidizing the second (3β)-cholest-5-en-3-sulfate organic cationic salt includes contacting the second (3β)-cholest-5-en-3-sulfate organic cationic salt with an oxidizing agent and at least one ketone in the presence of at least one surfactant.

In some instances, the at least one ketone is chosen from tetrahydrothiopyran-4-one 1,1-dioxide and halogenated ketones. In some cases, the halogenated ketones are chosen from 1,1,1-trifluoro-2-butanone, 4,4-difluorocyclohexanone, 2-2-2-4′-tetrafluoroacetophenone, and 1,1,1-trifluoroacetone. In certain cases, the at least one ketone is 1,1,1-trifluoro-2-butanone. The amount of ketone used relative to the oxidizing agent in the subject reaction may vary, and may be 1 equivalent or more, such as 2 equivalents or more, such as 3 equivalents or more, such as 4 equivalents or more, such as 5 equivalents or more, such as 6 equivalents or more, such as 7 equivalents or more, such as 8 equivalents or more, such as 9 equivalents or more, such as 10 equivalents or more, such as 15 equivalents or more, such as 20 equivalents or more, such as 25 equivalents or more, such as 30 equivalents or more, such as 35 equivalents or more, and including 50 equivalents or more of the ketone, and may range from 1 equivalent to 50 equivalents, such as 1 equivalent to 35 equivalents, 1 equivalent to 25 equivalents, 1 equivalent to 15 equivalents, 1 equivalent to 10 equivalents, 1 equivalent to 8 equivalents, 1 equivalent to 5 equivalents, 2 equivalent to 50 equivalents, 2 equivalent to 35 equivalents, 2 equivalent to 25 equivalents, 2 equivalent to 15 equivalents, 2 equivalent to 10 equivalents, 2 equivalent to 8 equivalents, 2 equivalent to 5 equivalents, 4 equivalent to 50 equivalents, 4 equivalent to 35 equivalents, 4 equivalent to 25 equivalents, 4 equivalent to 15 equivalents, 4 equivalent to 10 equivalents, 4 equivalent to 8 equivalents, 1 equivalent to 50 equivalents, 2 equivalent to 25 equivalents, or 5 equivalents to 10 equivalents.

In certain cases, the ketone is further purified before use. For example, the ketone may be purified by distillation prior to use. In some instances, the reactivity of the ketone is tested (e.g., tested for impurities by ¹H-NMR) in order to determine whether purification may be required.

In certain cases, oxidizing the second (3β)-cholest-5-en-3-sulfate organic cationic salt includes contacting the second (3β)-cholest-5-en-3-sulfate organic cationic salt with an oxidizing agent and at least one ketone in the presence of at least one surfactant and water. The amount of water present may vary, ranging from 0.0000001% w/v or more of the reaction mixture, such as 0.000001% w/v or more, such 0.00001% w/v or more, such as 0.0001% w/v or more, such as 0.001% w/v, such as 0.01% w/v or more, such as 0.1% w/v, such as 0.05% w/v or more, such as 0.1% w/v or more, such as 0.5% w/v or more, such as 1% w/v or more, such as 5% w/v or more, such as 10% w/v or more, such as 15% w/v or more, and including 25% w/v or more of the reaction mixture, and may range from 0.0000001% w/v to 25% w/v, such as 0.0000001% w/v to 15% w/v, 0.0000001% w/v to 10% w/v, 0.0000001% w/v to 5% w/v, 0.0000001% w/v to 1% w/v, 0.001% w/v to 25% w/v, 0.001% w/v to 15% w/v, 0.001% w/v to 10% w/v, 0.001% w/v to 5% w/v, 0.001% w/v to 1% w/v, 0.1% w/v to 25% w/v, 0.1% w/v to 15% w/v, 0.1% w/v to 10% w/v, 0.1% w/v to 5% w/v, 0.1% w/v to 1% w/v, 1% w/v to 25% w/v, 1% w/v to 15% w/v, 1% w/v to 10% w/v, 1% w/v to 5% w/v, 0.1% w/v to 50% w/v, 0.1% w/v to 10% w/v, or 0.5% w/v to 1% w/v.

The second (3β)-cholest-5-en-3-sulfate organic cationic salt may be oxidized at a temperature that ranges from −25° C. to 50° C., such as from −20° C. to 45° C., such as from −15° C. to 40° C., such as from −10° C. to 35° C., such as from −5° C. to 30° C., such as from −1° C. to 25° C., and including from 0° C. to 15° C. In certain cases, the second (3β)-cholest-5-en-3-sulfate organic cationic salt is oxidized at a temperature of from 0° C. to 5° C. Where the reaction mixture includes an amount of water, the reaction may be conducted at a temperature that is from −10° C. to 50° C., such as from −5° C. to 45° C., such as from 0° C. to 40° C., such as from 0° C. to 35° C., such as from 0° C. to 30° C., such as from 0° C. to 25° C., such as from 0° C. to 20° C., such as from 0° C. to 15° C., and including from 0° C. to 10° C.

The second (3β)-cholest-5-en-3-sulfate organic cationic salt may be oxidized at a pH that ranges from 5 to 7.5, such as a pH of from 5.5 to 7.0 and including a pH of from 5.5 to 6.5. In some cases, where the reaction mixture contains water (e.g., in a biphasic solvent system), the pH ranges from 5.0 to 6.0, such as a pH of from 5.0 to 5.9, such as a pH of from 5.0 to 5.8, such as a pH of from 5.0 to 5.7, such as a pH from 5.0 to 5.6, and including a pH of from 5.0 to 5.5.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours.

In some instances, the second (3β)-cholest-5-en-3-sulfate organic cationic salt is contacted in situ with a composition having potassium peroxymonosulfate and at least one ketone in the presence of at least one surfactant. In some cases, methods include contacting the potassium peroxymonosulfate with at least one ketone in the presence of at least one surfactant to form a separate oxidative reactive mixture and adding the oxidative reactive mixture to the second (3β)-cholest-5-en-3-sulfate organic cationic salt. In these cases, the potassium peroxymonosulfate may be contacted with the at least one ketone in the presence of the at least one surfactant for a duration of 0.1 minute or more before contacting the oxidative reactive mixture with the second (3β)-cholest-5-en-3-sulfate organic cationic salt, such as 1 minute or more, such as 2 minutes or more, such as 3 minutes or more, such as 5 minutes or more, and including 10 minutes or more, and the time may range from 2 minutes to 180 minutes, such as 3 minutes to 120 minutes or 4 minutes to 60 minutes. In certain instances, the potassium peroxymonosulfate may be contacted with the at least one ketone in the presence of the at least one surfactant to form a separate oxidative reactive mixture and immediately contacting the oxidative reactive mixture with the second (3β)-cholest-5-en-3-sulfate organic cationic salt. The oxidative reactive mixture may be formed at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C. and including from 0° C. to 15° C. Where the oxidative reactive mixture is not immediately contacted with the second (3β)-cholest-5-en-3-sulfate organic cationic salt, the oxidative reactive mixture may be maintained at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C., and including from 0° C. to 15° C.

In some cases, methods further include adding the oxidative reactive mixture to the second (3β)-cholest-5-en-3-sulfate organic cationic salt. In some instances, methods include adding dropwise the oxidative reactive mixture to the second (3β)-cholest-5-en-3-sulfate organic cationic salt. In some instances, the oxidative reactive mixture is added to the second (3β)-cholest-5-en-3-sulfate organic cationic salt in metered amounts. The metered amounts may be added continuously or at predetermined time intervals (e.g., every 30 seconds, 1 minute, 2 minutes, 3 minutes, 5 minutes, or some other interval). In some instances, the oxidative reactive mixture is added to the second (3β)-cholest-5-en-3-sulfate organic cationic salt by controlled addition, such as with a mechanically or computer controlled pump, e.g., syringe pump. In some cases, methods include generating the oxidative reactive mixture and adding a composition containing the second (3β)-cholest-5-en-3-sulfate organic cationic salt to the oxidative reactive mixture. In some instances, methods include adding dropwise the second (3β)-cholest-5-en-3-sulfate organic cationic salt to the oxidative reactive mixture. In some instances, the second (3β)-cholest-5-en-3-sulfate organic cationic salt is added to the oxidative reactive mixture in metered amounts. The metered amounts may be added continuously or at predetermined time intervals (e.g., every 30 seconds, 1 minute, 2 minutes, 3 minutes, 5 minutes, or some other interval). In some instances, the second (3β)-cholest-5-en-3-sulfate organic cationic salt is added to the oxidative reactive mixture by controlled addition, such as with a mechanically or computer-controlled pump, e.g., syringe pump.

In certain cases, oxidizing the second (3β)-cholest-5-en-3-sulfate organic cationic salt includes contacting the second (3β)-cholest-5-en-3-sulfate organic cationic salt with at least one oxidative species. In some instances, the at least one oxidative species is chosen from dioxiranes. In some instances, the dioxiranes are generated in situ in a composition having the second (3β)-cholest-5-en-3-sulfate organic cationic salt. In some instances, the dioxiranes are generated separately (e.g., in a separate reaction container, e.g., flask) and added to the composition having the second (3β)-cholest-5-en-3-sulfate organic cationic salt.

In certain cases, the second (3β)-cholest-5-en-3-sulfate organic cationic salt is oxidized in the presence of at least one base. In certain cases, the at least one base is chosen from weak bases. In some cases, the at least one base is chosen from potassium hydrogen carbonate, sodium hydrogen carbonate, potassium phenoxide, sodium citrate buffer, sodium phosphate buffer, potassium formate and potassium acetate. In certain cases, the at least one base is potassium hydrogen carbonate. In some cases, the at least one base may be added to the reaction mixture over time, such as in metered amounts where the base is added at predetermined time intervals (e.g., every 30 seconds, 1 minute, 2 minutes, 3 minutes, 5 minutes, or some other interval). In some cases, the at least one base may be a composition having water where the base present in the composition may be 0.0000001% w/v or more of the composition, such as 0.000001% w/v or more, such as 0.00001% w/v or more, such as 0.0001% w/v or more, such as 0.001% w/v or more, such as 0.01% w/v or more, such as 0.05% w/v or more, such as 0.1% w/v or more, such as 0.5% w/v or more, such as 1% w/v or more, such as 5% w/v or more, such as 10% w/v or more, such as 15% w/v or more, and including 25% w/v or more of the composition, and may range from 0.0000001% w/v to 25% w/v, such as 0.0000001% w/v to 15% w/v, 0.0000001% w/v to 10% w/v, 0.0000001% w/v to 5% w/v, 0.0000001% w/v to 1% w/v, 0.001% w/v to 25% w/v, 0.001% w/v to 15% w/v, 0.001% w/v to 10% w/v, 0.001% w/v to 5% w/v, 0.001% w/v to 1% w/v, 0.1% w/v to 25% w/v, 0.1% w/v to 15% w/v, 0.1% w/v to 10% w/v, 0.1% w/v to 5% w/v, 0.1% w/v to 1% w/v, 1% w/v to 25% w/v, 1% w/v to 15% w/v, 1% w/v to 10% w/v, 1% w/v to 5% w/v, 0.1% w/v to 20% w/v, 0.2% w/v to 15% w/v, or 0.3% w/v to 10% w/v. In certain cases, the at least one base may be an aqueous potassium hydrogen carbonate composition.

In certain cases, the second (3β)-cholest-5-en-3-sulfate organic cationic salt is oxidized by contacting with oxone in the presence of cetyltrimethylammonium hydrogen sulfate (CTAHS) followed by adding trifluorobutanone and potassium hydrogen sulfate to form 25-hydroxy-(3β)-cholest-(5,6-epoxy)-3-sulfate organic cationic salt (Scheme IIC2).

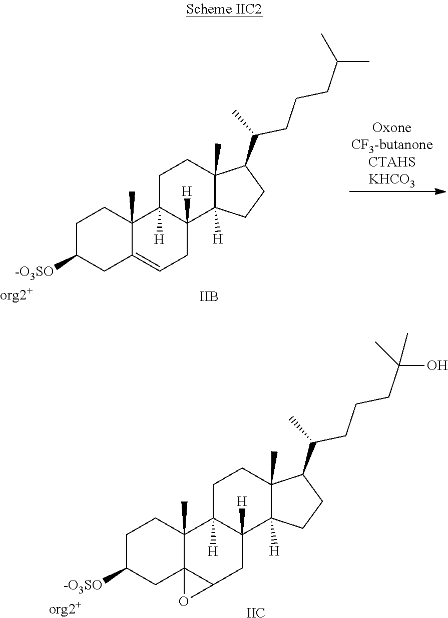

In certain instances, methods include forming an oxidative species in situ with the second (3β)-cholest-5-en-3-sulfate organic cationic salt, such as by contacting potassium peroxymonosulfate and trifluorobutanone in the presence of cetyltrimethylammonium hydrogen sulfate (CTAHS) in a reaction mixture with the second (3β)-cholest-5-en-3-sulfate organic cationic salt. In certain cases, forming an oxidative species in situ with the second (3β)-cholest-5-en-3-sulfate organic cationic salt includes forming a dioxirane in situ with the second (3β)-cholest-5-en-3-sulfate organic cationic salt.

In certain cases, methods include forming a dioxirane in a separate reaction and adding the dioxirane to the second (3β)-cholest-5-en-3-sulfate organic cationic salt. In these cases, the potassium peroxymonosulfate may be contacted with the trifluorobutanone in the presence of cetyltrimethylammonium hydrogen sulfate (CTAHS) for a duration of 0.1 minute or more before contacting the reactive composition with the second (3β)-cholest-5-en-3-sulfate organic cationic salt, such as 1 minute or more, such as 2 minutes or more, such as 3 minutes or more, such as 5 minutes or more, and including 10 minutes or more), and the time may range from 0.01 minutes to 120 minutes, such as 0.1 minutes to 90 minutes or 0.5 minutes to 60 minutes. In certain instances, the potassium peroxymonosulfate may be contacted with trifluorobutanone in the presence of cetyltrimethylammonium hydrogen sulfate (CTAHS) to form the oxidative reactive composition, which is immediately contacted with the second (3β)-cholest-5-en-3-sulfate organic cationic salt.

The 25-hydroxy-(3β)-cholest-(5,6-epoxy)-3-sulfate organic cationic salt may be deoxygenated to produce a 25-hydroxy-(3β)-cholest-5-en-3-sulfate organic cationic salt (Structure IID) (Scheme IID1).

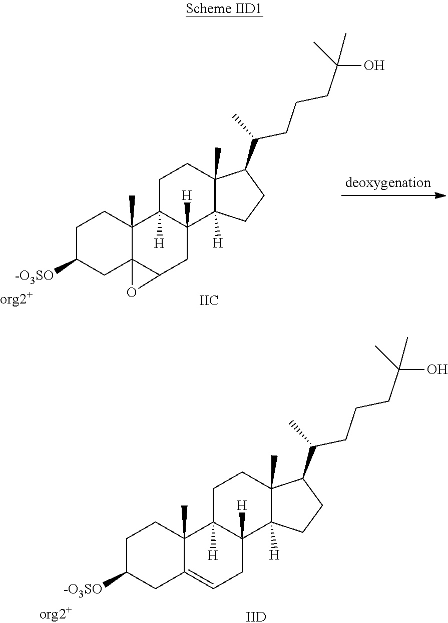

In some cases, generating 25-hydroxy-(3β)-cholest-5-en-3-sulfate organic cationic salt from the 25-hydroxy-(3β)-cholest-(5,6-epoxy)-3-sulfate organic cationic salt includes deoxygenation by contacting the 25-hydroxy-(3β)-cholest-(5,6-epoxy)-3-sulfate organic cationic salt with zinc. In certain instances, the 25-hydroxy-(3β)-cholest-(5,6-epoxy)-3-sulfate organic cationic salt is contacted with zinc in the presence of at least one halide and at least one acid. In some cases, the at least one halide is chosen from iodine and metal halides. In some cases, the metal halide is chosen from sodium iodide and lithium iodide. In some cases, the at least one acid is chosen from weak acids. In some cases, the at least one acid is chosen from acetic acid, hydrochloric acid, citric acid, para-toluene sulfonic acid, formic acid and methane sulfonic acid.

The amount of reagent used to deoxygenate the 25-hydroxy-(3β)-cholest-(5,6-epoxy)-3-sulfate organic cationic salt may vary, where in some instances, 0.0001 equivalents or more of reagent relative to the 25-hydroxy-(3β)-cholest-(5,6-epoxy)-3-sulfate organic cationic salt is used, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 20 equivalents, 1 equivalent to 10 equivalents, or 4 equivalents to 6 equivalents.

The 25-hydroxy-(3β)-cholest-(5,6-epoxy)-3-sulfate organic cationic salt may be deoxygenated at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C. and including from 0° C. to 25° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours.

In certain instances, methods include contacting the 25-hydroxy-(3β)-cholest-(5,6-epoxy)-3-sulfate organic cationic salt with zinc in the presence of iodine and acetic acid to generate the 25-hydroxy-(3β)-cholest-5-en-3-sulfate organic cationic salt (Scheme IID2).

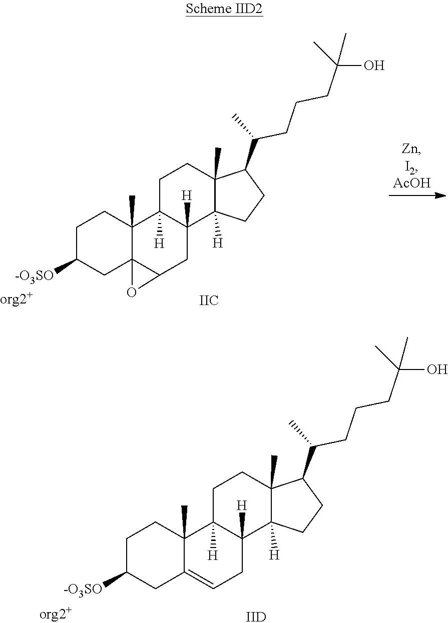

In some cases, the 25-hydroxy-(3β)-cholest-5-en-3-sulfate organic cationic salt (Structure IID) is contacted with a metal salt to produce the 25-hydroxy-(3β)-cholest-5-en-3-sulfate metal salt (Structure IIE) (Scheme IIE1).

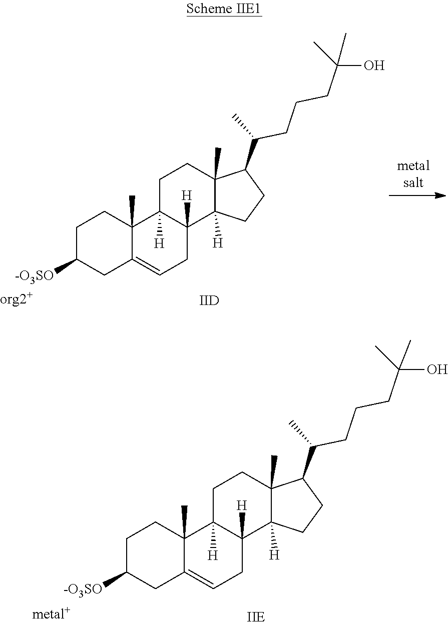

In some cases, methods to produce the 25-hydroxy-(3β)-cholest-5-en-3-sulfate metal salt include contacting the 25-hydroxy-(3β)-cholest-5-en-3-sulfate organic cationic salt with at least one sodium salt. In some cases, the at least one sodium salt is chosen from sodium acetate, sodium iodide, sodium chloride, sodium hydroxide and sodium methoxide. The 25-hydroxy-(3β)-cholest-5-en-3-sulfate organic cationic salt may be contacted with the metal salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C., such as from 0° C. to 45° C., such as from 5° C. to 40° C., and including from 10° C. to 35° C.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours. The amount of metal salt used relative to the 25-hydroxy-(3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 20 equivalents, 1 equivalent to 10 equivalents, or 1 equivalent to 7 equivalents.

In some cases, methods include contacting the 25-hydroxy-(3β)-cholest-5-en-3-sulfate pyridinium salt with sodium iodide to produce a 25-hydroxy-(3$P)-cholest-5-en-3-sulfate sodium salt (Scheme IIE2).

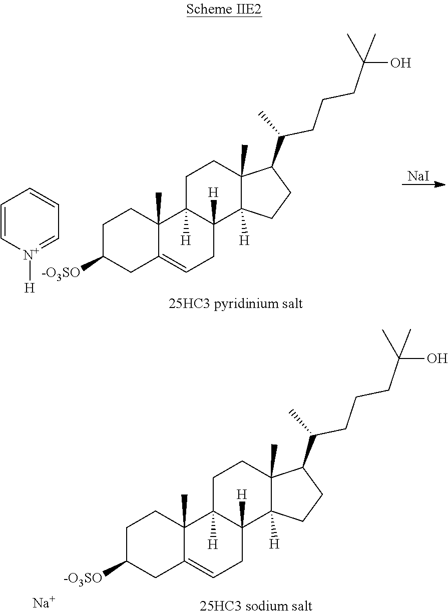

In some embodiments, the 25HC3S choline has relatively low solubility, which may be useful, e.g., in controlled release formulations such as injectable or oral controlled release formulations. As shown in the Examples, crystalline 25HC3S choline may be useful in controlled release formulations at least because of their low solubility in saline. Crystalline 25HC3S choline may also be useful in controlled release formulations at least because of its low solubility in Fasted State Simulated Gastric Fluid (FaSSGF).

In some embodiments, crystalline 25HC3S choline may be orally bioavailable. For example, salts of 25HC3S that have high solubility in Fasted State Simulated Intestinal Fluid (FaSSIF) may be orally bioavailable. As shown in the Examples, crystalline 25HC3S choline has relatively high solubility in FaSSIF. Salts of 25HC3 S that have high solubility in Fed State Simulated Intestinal Fluid (FeSSIF) may also be orally bioavailable. Crystalline 25HC3S choline has relatively high solubility in FeSSIF.

In some embodiments, salts of 25HC3S are non-hygroscopic, which facilitates handling of the drug substance at ambient conditions and avoids the need for special precautions, such as the need to handle in low humidity conditions, or handle in a dry environment, or keep in a tightly closed container. The manufacturing step of weighing these drug substance salts at ambient conditions is non-problematic, since there is no concern of the weighing changes on the balance due to moisture uptake. Also, the containers of these salts can be opened and closed multiple times at ambient conditions without the concerns of the powder changing composition due to water absorptions. The non-hygroscopic nature of these salts also allows for the preparation of wet granulations for oral tablet and capsule products, and minimizes the possibility for a polymorph or other solid-form conversion such as hydrate formation. For instance, as shown in the Examples, crystalline 25HC3S choline gains less than 0.5% water at 95% relative humidity. In addition, as shown in the DVS isotherms, when the crystalline 25HC3S choline gains small amounts of water as the relative humidity is increased to 95%, they reversibly lose all that water as the relative humidity is reduced to 5%.

In some embodiments, 25HC3 S choline is highly crystalline, which can be advantageous from a processing perspective, for example. Crystalline 25HC3S choline is highly crystalline. The XRPD patterns were successfully indexed by single unit cells and provide a robust description of the crystalline forms through tentative crystallographic unit cell parameters. The formula unit volumes from the indexing results are all consistent with anhydrous forms and the expected salt stoichiometry.

In some embodiments, the 25HC3S choline has a relatively high DSC (differential scanning calorimetry) endothermic transition (indicative of thermal degradation or solid state transformation). While not wishing to be bound by theory, this property may allow for dry heat sterilization (e.g., 160° for 2 hours) of the drug substance, to facilitate preparation of sterilized dosage forms. For instance, as shown in the Examples, the first significant endothermic transition for choline is near 198° C., indicating that it may be sterilized by dry heat processing.

In some embodiments, the 25HC3S choline has good temperature stability. As shown in the Examples, crystalline 25HC3S choline has good temperature stability.

In some embodiments, the choline counterion of 25HC3S choline may have beneficial effects in vivo. For instance, the choline salt may be beneficial because choline deficiency has also been implicated in such conditions related to fat accumulation and inflammation, with choline supplementation being suggested as potentially desirable in the treatment and/or management of such conditions (see, e.g., Zeisel et al. Nutr Rev. 2009 November; 67(11): 615-623, Corbin et al. Curr Opin Gastroenterol. 2012 March; 28(2): 159-165).

### CLAUSES

Clause 1. 25HC3S choline.
Clause 2. Crystalline 25HC3S choline.
Clause 3. The crystalline 25HC3S choline of clause 2, having an x-ray powder diffraction pattern comprising a peak at about 3.9° 2θ.
Clause 4. The crystalline 25HC3S choline of clause 2, having an x-ray powder diffraction pattern comprising a peak at about 7.8° 2θ.
Clause 5. The crystalline 25HC3S choline of clause 2, having an x-ray powder diffraction pattern comprising a peak at about 9.5° 2θ.
Clause 6. The crystalline 25HC3S choline of clause 2, having an x-ray powder diffraction pattern comprising a peak at about 10.1° 2θ.
Clause 7. The crystalline 25HC3S choline of clause 2, having an x-ray powder diffraction pattern comprising a peak at about 11.0° 2θ.
Clause 8. The crystalline 25HC3S choline of clause 2, having an x-ray powder diffraction pattern comprising a peak at about 12.2° 2θ.
Clause 9. The crystalline 25HC3S choline of clause 2, having an x-ray powder diffraction pattern comprising a peak at about 13.7° 2θ.
Clause 10. The crystalline 25HC3S choline of clause 2, having an x-ray powder diffraction pattern comprising a peak at about 14.7° 2θ.
Clause 11. The crystalline 25HC3S choline of clause 2, having an x-ray powder diffraction pattern comprising a peak at about 15.1° 2θ.
Clause 12. The crystalline 25HC3S choline of clause 2, having an x-ray powder diffraction pattern comprising a peak at about 15.8° 2θ.
Clause 13. The crystalline 25HC3S choline of clause 2, having an x-ray powder diffraction pattern comprising a peak at about 16.3° 2θ.
Clause 14. The crystalline 25HC3S choline of clause 2, having an x-ray powder diffraction pattern comprising a peak at about 19.1° 2θ.
Clause 15. The crystalline 25HC3S choline of clause 3, having an x-ray powder diffraction pattern further comprising a peak at about 7.8° 2θ.
Clause 16. The crystalline 25HC3S choline of clause 3, having an x-ray powder diffraction pattern further comprising a peak at about 9.5° 2θ.
Clause 17. The crystalline 25HC3S choline of clause 3, having an x-ray powder diffraction pattern further comprising a peak at about 10.1°2θ.
Clause 18. The crystalline 25HC3S choline of clause 3, having an x-ray powder diffraction pattern further comprising a peak at about 11.0020.
Clause 19. The crystalline 25HC3S choline of clause 3, having an x-ray powder diffraction pattern further comprising a peak at about 12.2° 2θ.
Clause 20. The crystalline 25HC3S choline of clause 3, having an x-ray powder diffraction pattern further comprising a peak at about 13.7° 2θ.
Clause 21. The crystalline 25HC3S choline of clause 3, having an x-ray powder diffraction pattern further comprising a peak at about 14.7° 2θ.
Clause 22. The crystalline 25HC3S choline of clause 3, having an x-ray powder diffraction pattern further comprising a peak at about 15.1°2θ.
Clause 23. The crystalline 25HC3S choline of clause 3, having an x-ray powder diffraction pattern further comprising a peak at about 15.8° 2θ.
Clause 24. The crystalline 25HC3S choline of clause 3, having an x-ray powder diffraction pattern further comprising a peak at about 16.3° 2θ.
Clause 25. The crystalline 25HC3S choline of clause 3, having an x-ray powder diffraction pattern further comprising a peak at about 19.1°2θ.
Clause 26. The crystalline 25HC3S choline of clause 15, having an x-ray powder diffraction pattern further comprising a peak at about 9.5° 2θ.
Clause 27. The crystalline 25HC3S choline of clause 15, having an x-ray powder diffraction pattern further comprising a peak at about 10.1°2θ.
Clause 28. The crystalline 25HC3S choline of clause 15, having an x-ray powder diffraction pattern further comprising a peak at about 11.0020.
Clause 29. The crystalline 25HC3S choline of clause 15, having an x-ray powder diffraction pattern further comprising a peak at about 12.2° 2θ.
Clause 30. The crystalline 25HC3S choline of clause 15, having an x-ray powder diffraction pattern further comprising a peak at about 13.7° 2θ.
Clause 31. The crystalline 25HC3S choline of clause 15, having an x-ray powder diffraction pattern further comprising a peak at about 14.7° 2θ.
Clause 32. The crystalline 25HC3S choline of clause 15, having an x-ray powder diffraction pattern further comprising a peak at about 15.1° 2θ.
Clause 33. The crystalline 25HC3S choline of clause 15, having an x-ray powder diffraction pattern further comprising a peak at about 15.8° 2θ.
Clause 34. The crystalline 25HC3S choline of clause 15, having an x-ray powder diffraction pattern further comprising a peak at about 16.3° 2θ.
Clause 35. The crystalline 25HC3S choline of clause 15, having an x-ray powder diffraction pattern further comprising a peak at about 19.1°2θ.
Clause 36. The crystalline 25HC3S choline of clause 26, having an x-ray powder diffraction pattern further comprising a peak at about 10.1°2θ.
Clause 37. The crystalline 25HC3S choline of clause 26, having an x-ray powder diffraction pattern further comprising a peak at about 11.0020.
Clause 38. The crystalline 25HC3S choline of clause 26, having an x-ray powder diffraction pattern further comprising a peak at about 12.2° 2θ.
Clause 39. The crystalline 25HC3S choline of clause 26, having an x-ray powder diffraction pattern further comprising a peak at about 13.7° 2θ.
Clause 40. The crystalline 25HC3S choline of clause 26, having an x-ray powder diffraction pattern further comprising a peak at about 14.7° 2θ.
Clause 41. The crystalline 25HC3S choline of clause 26, having an x-ray powder diffraction pattern further comprising a peak at about 15.1°2θ.
Clause 42. The crystalline 25HC3S choline of clause 26, having an x-ray powder diffraction pattern further comprising a peak at about 15.8° 2θ.
Clause 43. The crystalline 25HC3S choline of clause 26, having an x-ray powder diffraction pattern further comprising a peak at about 16.3° 2θ.
Clause 44. The crystalline 25HC3S choline of clause 26, having an x-ray powder diffraction pattern further comprising a peak at about 19.1°2θ.
Clause 45. The crystalline 25HC3S choline of clause 36, having an x-ray powder diffraction pattern further comprising a peak at about 11.0020.
Clause 46. The crystalline 25HC3S choline of clause 36, having an x-ray powder diffraction pattern further comprising a peak at about 12.2° 2θ.
Clause 47. The crystalline 25HC3S choline of clause 36, having an x-ray powder diffraction pattern further comprising a peak at about 13.7° 2θ.
Clause 48. The crystalline 25HC3S choline of clause 36, having an x-ray powder diffraction pattern further comprising a peak at about 14.7° 2θ.
Clause 49. The crystalline 25HC3S choline of clause 36, having an x-ray powder diffraction pattern further comprising a peak at about 15.1°2θ.
Clause 50. The crystalline 25HC3S choline of clause 36, having an x-ray powder diffraction pattern further comprising a peak at about 15.8° 2θ.
Clause 51. The crystalline 25HC3S choline of clause 36, having an x-ray powder diffraction pattern further comprising a peak at about 16.3° 2θ.
Clause 52. The crystalline 25HC3S choline of clause 36, having an x-ray powder diffraction pattern further comprising a peak at about 19.1°2θ.
Clause 53. The crystalline 25HC3S choline of clause 45, having an x-ray powder diffraction pattern further comprising a peak at about 12.2° 2θ.
Clause 54. The crystalline 25HC3S choline of clause 45, having an x-ray powder diffraction pattern further comprising a peak at about 13.7° 2θ.
Clause 55. The crystalline 25HC3S choline of clause 45, having an x-ray powder diffraction pattern further comprising a peak at about 14.7° 2θ.
Clause 56. The crystalline 25HC3S choline of clause 45, having an x-ray powder diffraction pattern further comprising a peak at about 15.1° 2θ.
Clause 57. The crystalline 25HC3S choline of clause 45, having an x-ray powder diffraction pattern further comprising a peak at about 15.8° 2θ.
Clause 58. The crystalline 25HC3S choline of clause 45, having an x-ray powder diffraction pattern further comprising a peak at about 16.3° 2θ.
Clause 59. The crystalline 25HC3S choline of clause 45, having an x-ray powder diffraction pattern further comprising a peak at about 19.1° 2θ.
Clause 60. The crystalline 25HC3S choline of clause 53, having an x-ray powder diffraction pattern further comprising a peak at about 13.7° 2θ.
Clause 61. The crystalline 25HC3S choline of clause 53, having an x-ray powder diffraction pattern further comprising a peak at about 14.7° 2θ.
Clause 62. The crystalline 25HC3S choline of clause 53, having an x-ray powder diffraction pattern further comprising a peak at about 15.1° 2θ.
Clause 63. The crystalline 25HC3S choline of clause 53, having an x-ray powder diffraction pattern further comprising a peak at about 15.8° 2θ.
Clause 64. The crystalline 25HC3S choline of clause 53, having an x-ray powder diffraction pattern further comprising a peak at about 16.3° 2θ.
Clause 65. The crystalline 25HC3S choline of clause 53, having an x-ray powder diffraction pattern further comprising a peak at about 19.1°2θ.
Clause 66. The crystalline 25HC3S choline of clause 60, having an x-ray powder diffraction pattern further comprising a peak at about 14.7° 2θ.
Clause 67. The crystalline 25HC3S choline of clause 60, having an x-ray powder diffraction pattern further comprising a peak at about 15.1°2θ.
Clause 68. The crystalline 25HC3S choline of clause 60, having an x-ray powder diffraction pattern further comprising a peak at about 15.8° 2θ.
Clause 69. The crystalline 25HC3S choline of clause 60, having an x-ray powder diffraction pattern further comprising a peak at about 16.3° 2θ.
Clause 70. The crystalline 25HC3S choline of clause 60, having an x-ray powder diffraction pattern further comprising a peak at about 19.1°2θ.
Clause 71. The crystalline 25HC3S choline of clause 66, having an x-ray powder diffraction pattern further comprising a peak at about 15.1°2θ.
Clause 72. The crystalline 25HC3S choline of clause 66, having an x-ray powder diffraction pattern further comprising a peak at about 15.8° 2θ.
Clause 73. The crystalline 25HC3S choline of clause 66, having an x-ray powder diffraction pattern further comprising a peak at about 16.3° 2θ.
Clause 74. The crystalline 25HC3S choline of clause 66, having an x-ray powder diffraction pattern further comprising a peak at about 19.1° 2θ.
Clause 75. The crystalline 25HC3S choline of clause 71, having an x-ray powder diffraction pattern further comprising a peak at about 15.8° 2θ.
Clause 76. The crystalline 25HC3S choline of clause 71, having an x-ray powder diffraction pattern further comprising a peak at about 16.3° 2θ.
Clause 77. The crystalline 25HC3S choline of clause 71, having an x-ray powder diffraction pattern further comprising a peak at about 19.1° 2θ.
Clause 78. The crystalline 25HC3S choline of clause 75, having an x-ray powder diffraction pattern further comprising a peak at about 16.3° 2θ.
Clause 79. The crystalline 25HC3S choline of clause 75, having an x-ray powder diffraction pattern further comprising a peak at about 19.1° 2θ.
Clause 80. The crystalline 25HC3S choline of clause 2, having an x-ray powder diffraction pattern comprising one or more of the following peaks at about 3.9° 2θ, about 7.8° 2θ, about 9.5° 2θ, about 10.1°2θ, about 11.0020, about 12.2° 2θ, about 13.7° 2θ, about 14.7° 2θ, about 15.1° 2θ, about 15.8° 2θ, about 16.3° 2θ, and about 19.1° 2θ.
Clause 81. The crystalline 25HC3S choline of clause 2, having an x-ray powder diffraction pattern comprising one or more of the following peaks at about 7.8° 2θ, about 9.5° 2θ, about 10.1° 2θ, about 11.0020, about 12.2° 2θ, about 13.7° 2θ, about 14.7° 2θ, about 15.1° 2θ, about 15.8° 2θ, about 16.3° 2θ, and about 19.1°2θ.
Clause 82. The crystalline 25HC3S choline of clause 2, having an x-ray powder diffraction pattern comprising one or more of the following peaks at about 9.5° 2θ, about 10.1°2θ, about 11.0020, about 12.2° 2θ, about 13.7° 2θ, about 14.7° 2θ, about 15.1°2θ, about 15.8° 2θ, about 16.3° 2θ, and about 19.1°2θ.
Clause 83. The crystalline 25HC3S choline of clause 2, having an x-ray powder diffraction pattern comprising one or more of the following peaks at about 10.1° 2θ, about 11.0020, about 12.2° 2θ, about 13.7° 2θ, about 14.7° 2θ, about 15.1°2θ, about 15.8° 2θ, about 16.3° 2θ, and about 19.1°2θ.
Clause 84. The crystalline 25HC3S choline of clause 2, having an x-ray powder diffraction pattern comprising one or more of the following peaks at about 11.0°2θ, about 12.2°2θ, about 13.7° 2θ, about 14.7° 2θ, about 15.1°2θ, about 15.8° 2θ, about 16.3° 2θ, and about 19.1°2θ.
Clause 85. The crystalline 25HC3S choline of clause 2, having an x-ray powder diffraction pattern comprising one or more of the following peaks at about 12.2°2θ, about 13.7°2θ, about 14.7° 2θ, about 15.1°2θ, about 15.8°2θ, about 16.3° 2θ, and about 19.1°2θ.
Clause 86. The crystalline 25HC3S choline of clause 2, having an x-ray powder diffraction pattern comprising one or more of the following peaks at about 13.7° 2θ, about 14.7° 2θ, about 15.1°2θ, about 15.8° 2θ, about 16.3° 2θ, and about 19.1°2θ.
Clause 87. The crystalline 25HC3S choline of clause 2, having an x-ray powder diffraction pattern comprising one or more of the following peaks at about 14.7° 2θ, about 15.1°2θ, about 15.8°2θ, about 16.3°2θ, and about 19.1°2θ.
Clause 88. The crystalline 25HC3S choline of clause 2, having an x-ray powder diffraction pattern comprising one or more of the following peaks at about 15.1°2θ, about 15.8° 2θ, about 16.3°2θ, and about 19.1°2θ.
Clause 89. The crystalline 25HC3S choline of clause 2, having an x-ray powder diffraction pattern comprising one or more of the following peaks at about 15.8°2θ, about 16.3°2θ, and about 19.1° 2θ.
Clause 90. The crystalline 25HC3S choline of clause 2, having an x-ray powder diffraction pattern comprising one or more of the following peaks at about 16.3°2θ and about 19.1°2θ.
Clause 91. The crystalline 25HC3S choline of clause 2, having an x-ray powder diffraction pattern comprising a peak at about 19.1° 2θ.
Clause 92. The crystalline 25HC3S choline of clause 2, having an x-ray powder diffraction pattern substantially the same as that found in FIG. 1.
Clause 93. The crystalline 25HC3S choline of clauses 2-92, having an orthorhombic unit cell.
Clause 94. The crystalline 25HC3S choline of clauses 2-93, having a unit cell with lengths of about 7.9 Å, about 9.5 Å, and about 45.1 Å.
Clause 95. The crystalline 25HC3S choline of clauses 2-94, wherein the water uptake by the crystalline choline salt is less than 0.5% by weight between a relative humidity range of about 5% to about 95%.
Clause 96. The crystalline 25HC3S choline of clause 2, having an x-ray powder diffraction pattern comprising peaks at about 3.9° 2θ, about 7.8° 2θ, about 9.5°2θ, about 10.1° 2θ, about 11.0° 2θ, about 12.2° 2θ, about 13.7° 2θ, about 14.7° 2θ, about 15.1° 2θ, about 15.8° 2θ, and about 16.3° 2θ.
Clause 97. The crystalline 25HC3S choline of clause 96, further comprising a peak at about 19.1°2θ.
Clause 98. The crystalline 25HC3S choline of clause 2, having an x-ray powder diffraction pattern comprising peaks at about 3.9° 2θ, about 7.8° 2θ, about 9.5° 2θ, about 10.120, about 11.0020, about 12.2° 2θ, about 13.7° 2θ, about 14.7° 2θ, about 15.1° 2θ, about 15.8° 2θ, about 16.3° 2θ, and about 19.1°2θ.
Clause 99. Substantially pure crystalline 25HC3S choline.
Clause 100. The substantially pure crystalline choline salt of clauses 2-98.
Clause 101. A process of preparing a salt of 25HC3S choline comprising the steps of preparing a solution of 25HC3S and treating the solution with a choline compound.
Clause 102. The process of clause 101, wherein the solution is an organic solution.
Clause 10³. The process of clause 102, wherein the organic solution comprises acetonitrile.
Clause 104. The process of clauses 101-10³, wherein the choline compound is choline hydroxide.
Clause 105. The process of clauses 100-104, wherein the 25HC3S solution is prepared by dissolving a salt of 25HC3S in an alcohol solvent.
Clause 106. The process of clause 105, wherein the salt of 25HC3S is a triethylammonium salt of 25HC3S.
Clause 107. The process of clause 106, wherein the triethylammonium salt of 25HC3S is made by the process comprising dissolving 25HC3S sodium in a suitable solvent and treating with a solution comprising triethylammonium hydrochloride and isolating a solid of 25HC3S triethylammonium salt.
Clause 108. The process of clause 107, wherein the suitable solvent comprises methanol.
Clause 109. The process of clauses 107-108, wherein the solution comprising triethylammonium hydrochloride further comprises triethylamine.
Clause 110. A crystalline 25HC3S choline made by the process of clauses 101-109.
Clause 111. A pharmaceutical composition comprising 25HC3S choline of any one of clauses 1-100 and 110, and at least one pharmaceutically acceptable excipient.
Clause 112. A method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, and conditions related to fat accumulation and inflammation, comprising administering to a patient in need thereof an effective amount of a compound of 25HC3S choline of any one of clauses 1-100 and 110-111.
Clause 113. The pharmaceutical composition of clause 111 configured for oral administration.
Clause 114. The method of clause 112, wherein the treatment administered.
Clause 115. 25HC3S choline of any one of any one of clauses 1-100, 110, 111, or 113 for use as a medicament.
Clause 116. 25HC3S choline of any one of any one of clauses 1-100, 110, 111, or 113 for use in a method as defined in clauses 112 or 114.
Clause 117. Use of 25HC3S choline of any one of clauses 1-100, 110, 111, or 113 in the manufacture of a medicament for use in a method as defined in any one of clauses 112 or 114.
Clause 118. The crystalline 25HC3S choline of any one of clauses 2-100, 110, or 115-116.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### General Synthetic Procedures for Preparing 25-hydroxy-(3β)-cholest-5-en-3-sulfate

25HC3S may be prepared by various methods. Enclosed herein are exemplary methods of making 25HC3 S. It should be noted that the methods deployed herein were not necessarily used during the synthesis of 25HC3S choline or crystalline 25HC3S choline described herein. However, they could be so used in such preparations.

All temperatures are in degrees Celsius (° C.) and are uncorrected. Reagent grade chemicals and anhydrous solvents were purchased from commercial sources and, unless otherwise mentioned, used without further purification. The names of the products were determined using the naming software included in Biovia electronic lab notebook. Silica gel chromatography was performed on Teledyne Isco instruments using pre-packaged disposable SiO₂ stationary phase columns with eluent flow rates of 15 to 200 mL/min. The analytical HPLC chromatograms were performed using an Agilent 1100 series instrument with DAD detector (190 nm to 300 nm). The mass spectra were recorded with a Waters Micromass ZQ detector at 130° C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX (5 μM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05), and EVO C₁₈ (5 μM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks which, in ¹H NMR, appear at 7.26 ppm for CDCl₃, 2.50 ppm for DMSO-d₆, and 3.31 ppm for CD₃OD.

### Example 1. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[alphenanthren-3-yl]sulfate

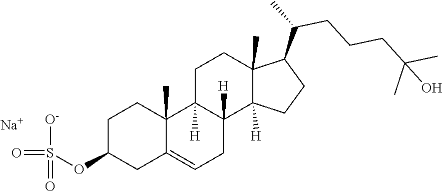

A dry 3-necked flask was charged with pyridine sulfur trioxide complex (12.45 g, 78 mmol), and the solid was suspended in toluene (1.5 L) and acetic anhydride (7.2 mL, 74.5 mmol). The mixture was stirred at 20° C. for 40 min, and pyridine (60 mL, 745 mmol) was added. The mixture was stirred at 20° C. for 20 min. (3S,8S,9S,10R,13R,14S,17R)-17-[(R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (30 g, 74.5 mmol) was added in a single portion as a solid. The mixture was stirred at 20° C. for 23 h. Aqueous sodium acetate solution (10 wt %, 123 mL, 149 mmol) was added dropwise with vigorous stirring over 5 min. The resultant mixture was stirred at 20° C. for 1 h. The solvent was pumped out of the reactor, collecting any solids onto a glass frit. ACN (700 mL) was added, and the slurry was stirred vigorously for 3 h. The slurry was pumped out of the reactor onto the same frit, and the remaining solids in the reactor were again suspended in ACN (700 mL) and stirred for 1 h before pumping out of the reactor to the glass frit. The solids in the frit were rinsed with diethyl ether (750 mL) and then suspended in DMF (800 mL). The mixture was stirred for 1 h at 20° C. The suspension was filtered, and the filtrate collected. To the filtrate, with stirring, was added diethyl ether (3.2 L). The resulting solids were collected by vacuum filtration, and the filter cake rinsed with diethyl ether (1 L). The solids were dried under reduced pressure to provide the title compound as a solid (15 g, 40%). ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES− [M]⁻ 481.3; LCMS (B05); t_{R}=1.18 m.

### Example 2. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[alphenanthren-3-yl]sulfate

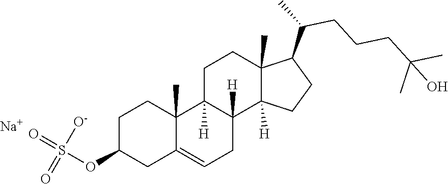

A dry 3-necked flask was charged with sulfur trioxide pyridine complex (4.74 g, 29.8 mmol). The solid was suspended in toluene (500 mL), and acetic anhydride (2.61 mL, 27.67 mmol) was added in a single portion. The resultant mixture stirred at 23° C. for 1 h. Pyridine (20 mL, 248.4 mmol) was added, and the mixture was stirred at 23° C. for 5 min. (3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (10 g, 24.83 mmol) was added in a single portion as a solid. The mixture stirred at 23° C. for 23 h. The reaction was diluted with MeOH (2.01 mL, 49.7 mmol) and stirred at 23° C. for 1 h. The suspension was filtered, and the solids washed with toluene (2×200 mL). The solids were collected and dried under high vacuum to provide a solid. The solids were partially dissolved in ACN (600 mL), and sodium iodide (14.9 g, 99.3 mmol) was added. The mixture was stirred at 23° C. for 10 min before being cooled to 0° C. with an ice-bath and stirred for 1.5 h. The suspension was filtered, and the solids washed with cold ACN (2×275 mL) and acetone (2×200 mL). The solids were collected and dried under high vacuum to provide the title compound as a solid (7.24 g, 57%). ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES− [M]⁻ 481.3; LCMS (B05); t_{R}=1.18 m.

### Example 3. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[alphenanthren-3-yl]sulfate

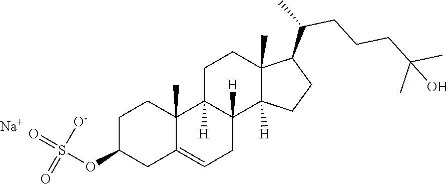

A 15 L jacketed reactor was heated to 60° C. and purged with nitrogen for 1.5 h. The jacket temperature was set to 30° C. and 2-MeTHF (7 L) was charged. (3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (495 g, 1.23 mol) was charged, and the manway/glassware was rinsed with 2-MeTHF (6 L). The solution was cooled to 25° C., additional 2-MeTHF (1 L) was added, and sulfur trioxide pyridine complex (234.8 g, 1.47 mol) was added. The mixture was stirred at 28° C. for 24 h. 2-MeTHF (2 L) was added, the mixture was stirred for a further 16 h, cooled to 20° C. and filtered. The solids were rinsed with 2-MeTHF (3.5 L). The solids were taken up in a solution of NaOH (118 g, 2.95 mmol) in MeOH (6 L). The mixture was stirred at 25° C. for 1 h and then filtered on a plug of Celite. The filtrate was concentrated to 3.5 L and diluted with diethyl ether (8 L). The suspension was chilled to 15° C. and filtered to provide the title compound as a solid (146.8 g, 24%). The filtrate was concentrated to 1 L and again mixed with diethyl ether (4 L). The solids were collected by vacuum filtration to provide the title compound as a solid (68.5 g, 11%). The Celite was extracted with MeOH (2 L), which was concentrated to 500 mL and diluted with diethyl ether (3 L) and the solids were collected by vacuum filtration to provide the title compound as a solid (53.3 g, 8.6%). A fourth crop was isolated from the filtrates (11.88 g, 2%). Total yield: 280.5 g, 45%. ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES− [M]⁻ 481.3; LCMS (B05); t_{R}=1.18 m.

### Example 4. Synthesis of Ammonium [(3S,10R,13R,17R)-17-[(1R)-5-hydroxy-1,5-dimethyl-hexyl]-10,13-dimethyl-2,3,4,7,8,9,11,12,14,15,16,17-dodecahdro-1H-cyclopenta[alphenanthren-3-yl] sulfate

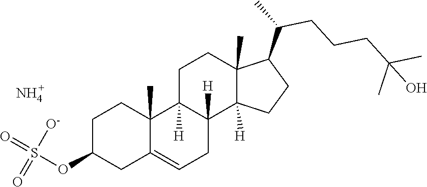

Sulfur trioxide dimethyl formamide complex (42 mg, 0.273 mmol) was added to a stirred solution of (3S,10R,13R,17R)-17-(5-hydroxy-1,5-dimethyl-hexyl)-10,13-dimethyl-2,3,4,7,8,9,11,12,14,15,16,17-dodecahydro-1H-cyclopenta[a]phenanthren-3-ol (100 mg, 0.25 mmol) in anhydrous DCM (20 mL) at 0° C. The mixture was stirred at 0° C. for 5 h, and then the reaction was warmed to 20° C. The mixture was concentrated under reduced pressure to afford a crude solid which was purified by column chromatography on silica gel (12 g cartridge) eluting with mixtures of DCM and MeOH (0-20%) to afford impure title compound. m/z: ES− [M−H]⁻ 481.

### Example 5. Synthesis of Pyridin-1-ium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[alphenanthren-3-yl] sulfate

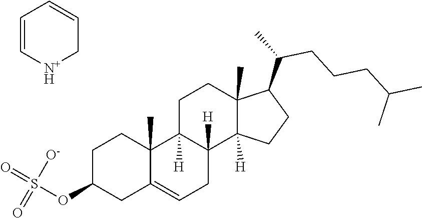

In an oven-dried round-bottom flask, sulfur trioxide pyridine complex (4.53 g, 28.5 mmol) was suspended in toluene (240 mL). Acetic anhydride (2.44 mL) was added, followed by pyridine (20.8 mL). The reaction was stirred at 23° C. for 1 h, and cholesterol (10 g, 25.9 mmol) was added in a single portion as a solid. The suspension was stirred at 23° C. for 18 h and, filtered on a glass frit, and the solids rinsed with toluene (100 mL) followed by hexanes (100 mL). The solids were suspended in chloroform (400 mL) and filtered on the same frit. The frit was rinsed with chloroform (200 mL) and the filtrate collected. The filtrate was diluted to 1.8 L with hexanes and refrigerated for 1 h. The suspension was filtered; the solids were rinsed with diethyl ether (100 mL) and dried under high vacuum to provide the title compound as a solid (10.06 g, 71%). ¹H NMR (500 MHz, MeOD) δ 8.89 (dd, J=6.6, 1.4 Hz, 2H), 8.79-8.61 (m, 1H), 8.27-8.05 (m, 2H), 5.38 (d, J=5.3 Hz, 1H), 4.13 (tt, J=11.5, 4.7 Hz, 1H), 2.53 (ddd, J=13.3, 5.0, 2.3 Hz, 1H), 2.43-2.28 (m, 1H), 2.12-2.02 (m, 2H), 2.01-1.94 (m, 1H), 1.94-1.80 (m, 2H), 1.70-0.83 (m, 20H), 1.03 (s, 3H), 0.95 (d, J=6.6 Hz, 3H), 0.88 (dd, J=6.6, 1.9 Hz, 6H), 0.72 (s, 3H); m/z: ES− [M]⁻ 465.3; LCMS (B05); t_{R}=1.40 m.

### Example 6. Synthesis of Pyridin-1-ium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[alphenanthren-3-yl] sulfate

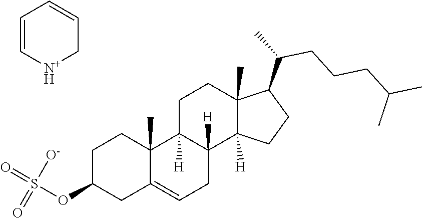

Cholesterol sulfate pyridinium salt was prepared by adding sulfur trioxide pyridine complex (4.53 g, 28.5 mmol) to a solution of cholesterol (10 g, 25.9 mmol) in 2-MeTHF (250 mL) at 30° C. and stirring the mixture for 16 h. The suspension was then filtered, and the solids rinsed with 2-MeTHF (50 mL) to afford the title compound.

### Example 7. Synthesis of Sodium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[alphenanthren-3-yl]sulfate

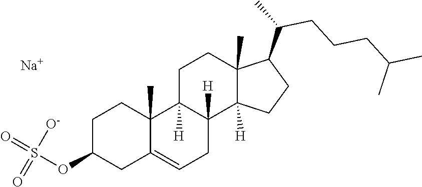

Chlorosulfonic acid (0.03 mL, 0.45 mmol) was added to a solution of 2,6-lutidine (0.08 mL, 0.69 mmol) in acetone (2.5 mL) over molecular sieves. The solution was stirred at 20° C. for 2 min before being cooled to 0° C. A solution of cholesterol (100 mg, 0.26 mmol) in acetone (5 mL), which was previously dried over molecular sieves, was added dropwise. The mixture stirred at 0° C. for 2 h before warming to 20° C. over 16 h. The mixture was filtered and the solid was collected. The solid was then suspended in acetone (10 mL) and aqueous sodium bicarbonate was added until bubbling subsided. The suspension was filtered and the solid triturated with MeOH (10 mL) and DCM (10 mL). The solvent was removed under reduced pressure to afford a solid. The solid was triturated with ACN (30 mL), filtered, and the filtrate was lyophilized to afford the title compound as a solid (7.3 mg, 5.8%). ¹H NMR (500 MHz, DMSO) δ 5.31-5.19 (m, 1H), 4.10 (s, 1H), 3.87-3.78 (m, 1H), 2.42-2.31 (m, 1H), 2.13 (dd, J=14.5, 7.6 Hz, 1H), 2.02-1.69 (m, 5H), 1.62-0.95 (m, 20H), 0.94 (s, 3H), 0.89 (d, J=6.5 Hz, 4H), 0.84 (dd, J=6.6, 2.5 Hz, 7H), 0.65 (s, 3H).

### Example 8. Synthesis of Ammonium [(3S,5S,8R,9S,10S,13R,14S,17R)-17-[(1R)-1,5-dimethylhexyl]-10,13-dimethyl-2,3,4,5,6,7,8,9,11,12,14,15,16,17-tetradecahydro-1H-cyclopenta[alphenanthren-3-yl]sulfate

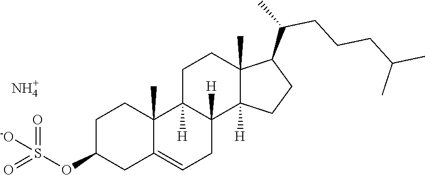

Sulfur trioxide pyridine complex (300 mg, 1.88 mmol) was added to a solution of cholestanol (300 mg, 0.772 mmol) in pyridine (5.00 mL), and the suspension was stirred at 20° C. for 16 h. The residue was purified by silica gel chromatography (24 g cartridge) with MeOH (5% NH₄OH) in DCM eluting with mixtures of DCM and MeOH (0-30%) to afford the title compound as a solid (314 mg, 84%). ¹H NMR (500 MHz, DMSO-d6) δ 7.08 (s, 4H), 3.97-3.86 (m, 1H), 1.91 (dd, J=12.5, 3.5 Hz, 1H), 1.86-1.71 (m, 2H), 1.69-1.55 (m, 3H), 1.55-1.41 (m, 3H), 1.38-1.25 (m, 5H), 1.25-0.90 (m, 15H), 0.88 (d, J=6.6 Hz, 4H), 0.84 (dd, J=6.6, 2.4 Hz, 7H), 0.74 (s, 3H), 0.62 (s, 3H); m/z: ES [M-NH₄]⁻ 467.3; HPLC (BEH Ambicarb/ACN 5-100%) t_{R}=7.48 min.

### Example 9. Synthesis of Ammonium [(3R,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[alphenanthren-3-yl] sulfate

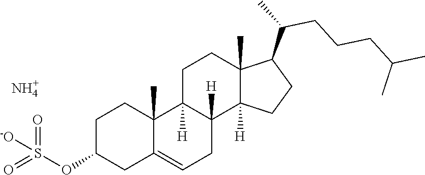

Sulfur trioxide pyridine complex (206 mg, 1.29 mmol) was added to a solution of (3R,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-ol (200 mg, 0.517 mmol) in pyridine (5.00 mL). The suspension was stirred at 20° C. for 16 h, then concentrated under reduced pressure. The residue was purified by silica gel chromatography (24.0 g cartridge) eluting with mixtures of DCM and 5% NH₄OH in MeOH (0-30%) to afford the title compound as a solid (160 mg, 64%). ¹H NMR (500 MHz, DMSO) δ 7.07 (s, 4H), 5.18-5.14 (m, 1H), 4.32-4.27 (m, 1H), 2.40-2.29 (m, 1H), 2.16 (dt, J=14.9, 2.4 Hz, 1H), 2.01-1.72 (m, 4H), 1.60-0.96 (m, 22H), 0.94 (s, 3H), 0.90 (d, J=6.5 Hz, 3H), 0.84 (dd, J=6.6, 2.4 Hz, 6H), 0.65 (s, 3H); m/z: ES [M-NH₄]⁻ 465.6; HPLC (BEH AmForm/ACN 5-100%) t_{R}=2.76 min.

### Example 10. Synthesis of Pyridinium [(3S,8S,9S,10R,13R,14S,17R)-17-[(1R)-5-hydroxy-1,5-dimethyl-hexyl]-10,13-dimethyl-2,3,4,7,8,9,11,12,14,15,16,17-dodecahydro-1H-cyclopenta[alphenanthren-3-yl]sulfate

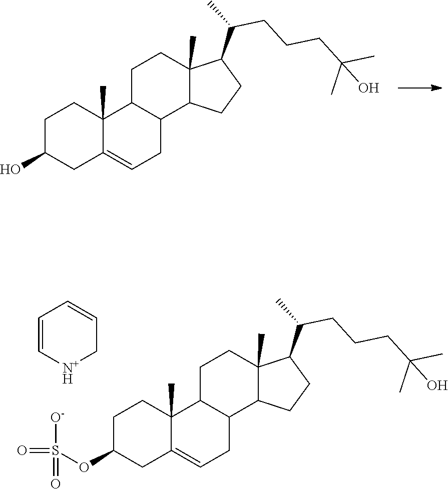

Acetic anhydride (0.0704 mL, 0.745 mmol) was added to a suspension of sulfur trioxide pyridine complex (125 mg, 0.782 mmol) in anhydrous toluene (15.0 mL). The suspension was stirred at 20° C. for 40 min, and pyridine (0.600 mL) was added. The suspension was stirred at 20° C. for 20 min. (3S,8S,9S,10R,13R,14S,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (300 mg, 0,745 mmol) was added in a single portion as a solid. The suspension was stirred at 20° C. for 20 h. The mixture was filtered on a glass frit to afford title compound as a solid (329 mg, 92% purity, 72% yield).

¹H NMR (500 MHz, DMSO-d₆) δ 8.99-8.88 (m, 2H), 8.65-8.53 (m, 1H), 8.13-7.97 (m, 2H), 5.30-5.20 (m, 1H), 3.93-3.71 (m, 1H), 2.41-2.32 (m, 1H), 2.18-2.08 (m, 1H), 2.02-1.71 (m, 5H), 1.59-0.95 (m, 20H), 1.05 (s, 6H), 0.94 (s, 3H), 0.90 (d, J=6.4 Hz, 3H), 0.65 (s, 3H); m/z ES⁺[M+H]⁺481.32; HPLC (DUR B) t_{R}=1.36 min.

### Example 11. Synthesis of 39-25-hydroxycholest-5-ene sulfate (1, as Sodium Salt)

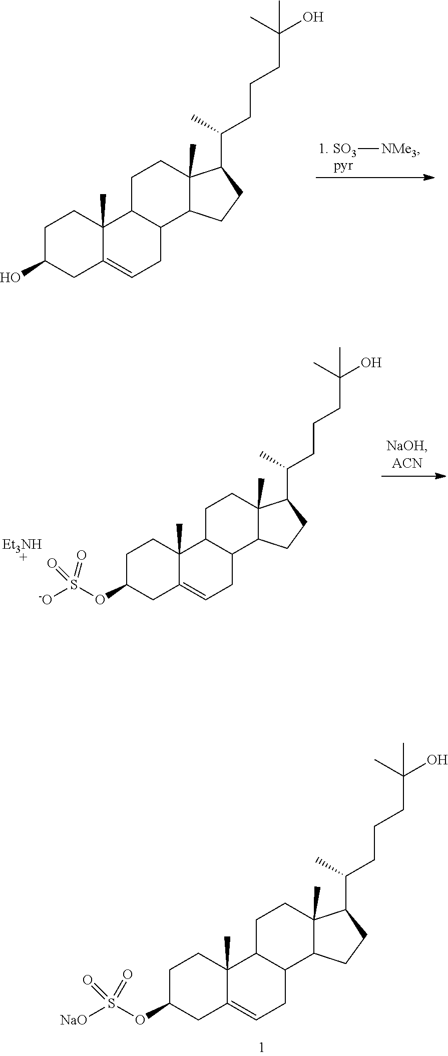

### Example 11A. Preparation of 3β-25-hydroxycholest-5-ene sulfate —Route 1

3β, 25-dihydroxycholest-5-ene (4.6 g, 0.011 mol) and triethylamine (1.7 ml, 0.023 mol) were suspended in pyridine (57 ml) and heated to 50° C. The sulfur trioxide trimethylamine complex (3.2 g, 0.023 mol) was added and the mixture agitated for 24 hours. A further charge of sulfur trioxide trimethylamine complex (0.77 g, 0.006 mol) was made and the mixture agitated for an additional 4 hours. With jacket at 50° C., the reaction mixture was distilled to−20% of initial volume. The residue was purified by silica gel chromatography (11 0 g), eluting with an ethyl acetate/methanol/triethylamine (90/9/1 v/v) mixture; fractions were analyzed by TLC (4:1 methylene chloride:methanol) using a phosphomolybdic acid stain. Fractions containing the 3- and 25-sulfate regioisomers were combined and evaporated (bath temp <35° C.). The residue (4.2 g, 0.0072 mol) was slurried in acetonitrile (25 g), treated with 1 N sodium hydroxide (7.2 ml, diluted from 30% sodium hydroxide solution) for 1 hour, and then filtered. Solids were rinsed through with aceotnitrile (25 g) and dried to a constant weight (2.77 g). The solids, containing a mixture of 3- and 25-sodium sulfate salts (2.77 g), were triturated with ethanol (27.7 g, 10S) at 50° C. for 1 hour and then filtered at 5° C. The isolated solids were dried to a constant weight (1.2 g). The solids (1.2 g) were suspended in 6:1 acetonitrile/water (10 S) at 30° C. for 30 minutes and then filtered. Filtration required about 40 minutes. Solids were dried to a constant weight (0.86 g) and analyzed.

### Example 11B. Preparation of 31-25-hydroxycholest-5-ene sulfate —Route 2—Excess sulfating agent

An excess of sulfur trioxide trimethylamine complex was used to drive the reaction toward formation of the disulfate. The 3-hydroxy group of 34, 25-dihydroxycholest-5-ene is about 6 times more reactive towards sulfation than the 25-hydroxyl. Providing excess sulfating agent and allowing the reaction to proceed to high conversion will provide monosulfate of higher regioisomeric purity. This result was observed during this synthesis. A solution of 3β, 25-dihydroxycholest-5-ene (4.1 g) in pyridine (75 ml) was vacuum distilled to reduce the volume to 50 ml. This was performed to remove isopropanol (from the recrystallization of the diol) and any moisture present. Triethylamine (2 equivalents) and then a total of 1.75 equivalents of sulfur trioxide trimethylamine complex was added in portions (1.0, 0.5 and 0.25 equivalents) to the reaction at 50° C. cover a period of 18 hours and the reaction was allowed run for a total of 43 hours. The reaction mixture was concentrated by vacuum distillation and the residue was absorbed onto SiO₂ (10 g). The loaded SiO₂ was placed on a SiO₂ column and eluted with 2-50% methanol/ethyl acetate/1% triethylamine. The appropriate fractions from the column were combined and evaporated to yield the disulfate (3.1 g, 39.7%) and the monosulfate (2.6 g, 44.7%). The monosulfate was obtained as a 22.1 mixture of the 3-sulfate and 25-sulfate. The solids were suspended in acetonitrile (25 g), treated with 1 N sodium hydroxide (4.44 ml), and then filtered. A thick gel formed, which was difficult to manipulate and was not filtered. The product was a suspension in acetonitrile/water. The solvents were removed by rotary evaporation at 40° C. and the residue was dried in a vacuum oven at 40° C. Trituration of the solid with acetone yielded a white solid: 1.27 g, 24.9%. This product showed only the 3β-sulfated product, but was contaminated with peaks at RRT 8.18 (unknown, 2.0%), RRT 15.17 (diol, 2.2%) and RRT 16.70 (unknown, 1.8%).

### Example 12. Preparatory Scale Synthesis of 3β-25-hydroxycholest-5-ene sulfate (1, as sodium salt

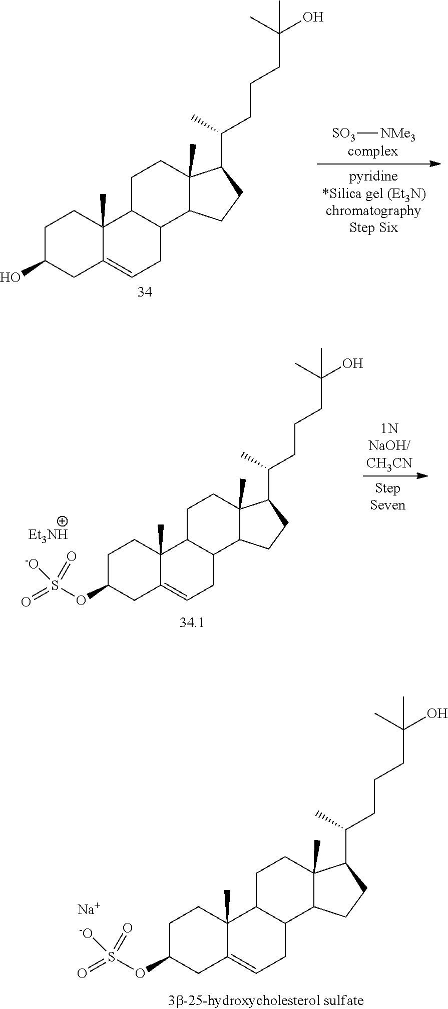

A 2 L, three-necked, round-bottomed flask with an overhead stirrer was charged with 3β, 25-dihydroxycholest-5-ene (34) (30 g, 74.5 mmol) and dry pyridine (500 mL, Sigma-Aldrich, cat. #270970-1L, lot #SHBC6287V). Sulfur trioxide-trimethylamine complex (12.2 g, 89.4 mmol, Sigma-Aldrich, cat. #135879-100G, lot #MKBH5585V) was added in one portion. The suspension was stirred at room temperature overnight. The reaction mixture was concentrated and the residue was purified by column chromatography to give 25.9 g (59%) of white solid as the triethylamine salt (HPLC: 98.6% purity). To a suspension of triethylamine salt 34.1 (64 g, 110.1 mmol) in ACN (1 L) was added 1 N NaOH (110 mL, 110.1 mmol, NaOH, Fisher, cat. #S318-3, lot #034906), and the mixture was stirred for 1 h at room temperature. The solid was filtered, washed with ACN (1 L), and dried under vacuum (P₂O₅) overnight, yield: 51.5 g, 93% (HPLC: 98.6% purity).

After overnight stirring, the reaction was a gel-like mixture. TLC showed the expected product as the major spot (TLC: 20% MeOH in DCM, R_{f}=0.4), with both the starting material (R_{f}>0.9) and 3b-25-hydroxycholesterol disulfate (R_{f}<0.1) as minor spots. Silica gel (1 kg, Sorbent Technologies, cat. #40930-2.5 kg) was packed to form a column of dimension 10 cm×42 cm. The column equilibration was accomplished with 1% triethylamine (Et₃N, Fisher, cat #04885-4, lot #062833) in DCM (2.8 L). The crude residue was dissolved in DCM (200 mL) and Et₃N (20 mL), which was directly loaded into the column. Triethylamine was used at this stage to avoid decomposition of the product and of the disulfate (which forms an olefin that is then very difficult to remove from the product). Initial elution was DCM (1% Et₃N) (2 L), followed by 1% MeOH in DCM (1% Et₃N)(1 L), 2% MeOH in DCM (1% Et₃N)((3 L), 5% MeOH in DCM (1% Et₃N)((1 L). The product began to elute in 2% MeOH in CH₂Cl₂ (1% Et₃N). The collected fractions were concentrated via rotary evaporation below 36° C. (if the temperature is higher than 45° C., decomposition of the product in the presence of MeOH is observed). Both TLC and NMR were checked for the selected fractions. HPLC (Zorbax SB-18, 4.6×150 mm, 5 μm, 202 nm, flow rate 0.8 mL/min): Solvent A: MeOH/5% ACN/7.4 mM NH₄OAc; Solvent B: H₂O/5% ACN/7.4 mM NH₄OAc. Gradient 75% A and 25% B to 100% A. Product: 98.6% purity; 1.4% (starting material 34). HPLC: Durashell C₁₈ (Agela Technologies, 4.6×50 mm, 3 mm, 100 Å); Solvent A: MeOH/5% ACN/7.4 mM NH₄OAc; Solvent B: H₂O/5% ACN/7.4 mM NH₄OAc. Product: 98.6% purity; 1.4% (starting material 34).

### Example 13. Large Scale Synthesis of 3β-25-hydroxycholest-5-ene sulfate (1, as Sodium Salt)

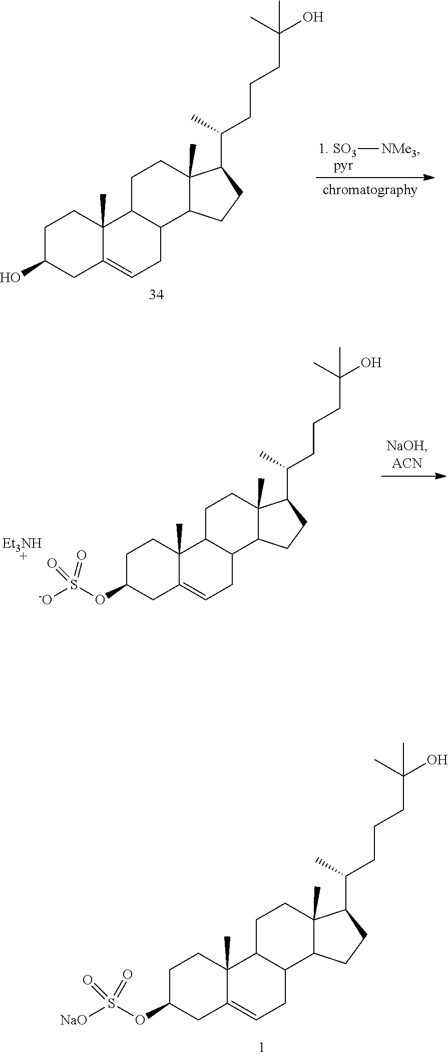

### Summary of Kilogram-Scale Preparation

3β, 25-dihydroxycholest-5-ene (34) (2.6 kg) and pyridine (39.2 kg) was combined and the mixture heated to 40° C. with agitation in two 50L reactors. Sulfur trioxide-trimethylamine (1.1 kg) was added to the mixture and stirred at 40° C. for 6-12 hours until the reaction was complete. The mixture was concentrated to minimum stir volume under vacuum distillation and then diluted with methylene chloride and triethylamine.

The crude reaction mixture in methylene chloride was loaded onto a 2.33 ft³ stainless steel column (C-105) packed with silican gel and eluted with methylene chloride (containing 1% methanol and 1% triethylamine). Fractions containing undesired product were collected in waste drums. Fractions containing desired product are collected and concentrated in the reactor.

Acetonitrile, water and sodium hydroxide were added to the reactor containing the desired product and the mixture was agitated until the reaction was deemed complete. The resulting slurry was cooled to 10-15° C. and filtered to isolate compound 1. The cake of isolated compound 1 was washed with acetonitrile and then dried at 40° C. under vacuum until a constant weight was achieved.

The solid was filtered, washed with acetonitrile (1 L), and dried under vacuum (P₂O₅) overnight, yield: 51.5 g, 93% (HPLC: 98.6% purity).

### DISCUSSION

Analysis of the reaction mixture by HPLC after 6 h showed 44.1% remaining starting material. The reaction was considered complete and distilled under vacuum to a minimum stir volume (Step 5.3). To the resulting thick residue were added methylene chloride and triethylamine, and the solution was transferred to a clean 5 gallon glass carboy. Thick solids precipitated in the glass carboy after holding the solution overnight. The solids were filtered away using the benchtop filter. Approximately ⅓ of the clear filtrate was charged to the top of a C-105 column. The silica in the C-105 column was previously flushed with ethyl acetate and methanol, and then equilibrated with 1% triethylamine in methylene chloride eluent.

Once the crude solution was loaded to the top of the column, eluent was charged to maintain a pressure of −10 psi. Eluent was sampled as it exited the column every 10-15 minutes. Pyridine and 3β, 25-dihydroxycholest-5-ene were present in the first two samples, but the 3β-triethylamine salt and 25-sulfate regioisomer were detected in the third sample in addition to pyridine and 3β, 25-dihydroxycholest-5-ene. Since minimal separation occurred, all remaining material was eluted from the column using the polar eluent (10% MeOH, 1% NEt3, and 98% DCM). The filtrate was concentrated and combined with the remaining two-thirds of the crude solution from the carboy. After distillation, the crude solution was transferred to a clean carboy. The eluent exiting the column was analyzed and contained 1.7% methanol (¹H NMR area %). The column was equilibrated with eluent (1% triethylamine in methylene chloride) and analyzed for methanol (0.25% methanol, ¹H NMR area %). Solids began to form in the carboy during this time. The slurry was filtered, and the filtrate was collected in a clean carboy.

Approximately one-third of the crude solution was loaded onto a second C-105 column. Eluent was charged to the column to maintain <5 psi. Analysis of the eluent leaving the column by thin layer chromatography (TLC) showed that separation was taking place. Once 3β, 25-dihydroxycholest-5-ene was no longer detected by TLC, the eluent was analyzed by ¹H NMR to ensure that the 3β-triethylamine salt was separated from the 25-sulfate regioisomer. A sample was removed from the eluent containing drum, and the purity of the 3β-triethylamine salt was 85% with 15% 25-sulfate regioisomer present (¹H NMR). An HPLC weight percentage assay showed that 127 g of 3β-triethylamine salt/25-sulfate regioisomer was collected in the drum (85% 3β-triethylamine salt). The purified material was set aside and the remaining two-thirds of the crude solution was purified by chromatography. The silica in the C-105 column was flushed with methanol and then equilibrated with 1% triethylamine in methylene chloride (0.2% methanol by ¹H NMR area % in the eluent after regeneration).

The solids that precipitated from the carboy were analyzed by ¹H NMR and identified as the quaternary ammonium salt produced from the reaction of methylene chloride with triethylamine (from the SO₃NMe₃ reagent) and methylene chloride with triethylamine. The methylene chloride-triethylamine complex was separated by filtration, while methylene chloride-triethylamine complex was formed in the chromatography. Formation of the salt occurred under ambient conditions and was rapid in certain instances in a pressurized environment. The establishment of an equilibrium in which the triethylammonium moiety of the 3β-triethylamine salt may be exchanging for the quaternary ammonium salt to give the quaternary ammonium complex and the triethylamine hydrochloride. Equilibrium favors the formation of the quaternary ammonium complex since there is more methylene chloride-triethylamine complex present. Triethylamine hydrochloride was isolated and characterized.

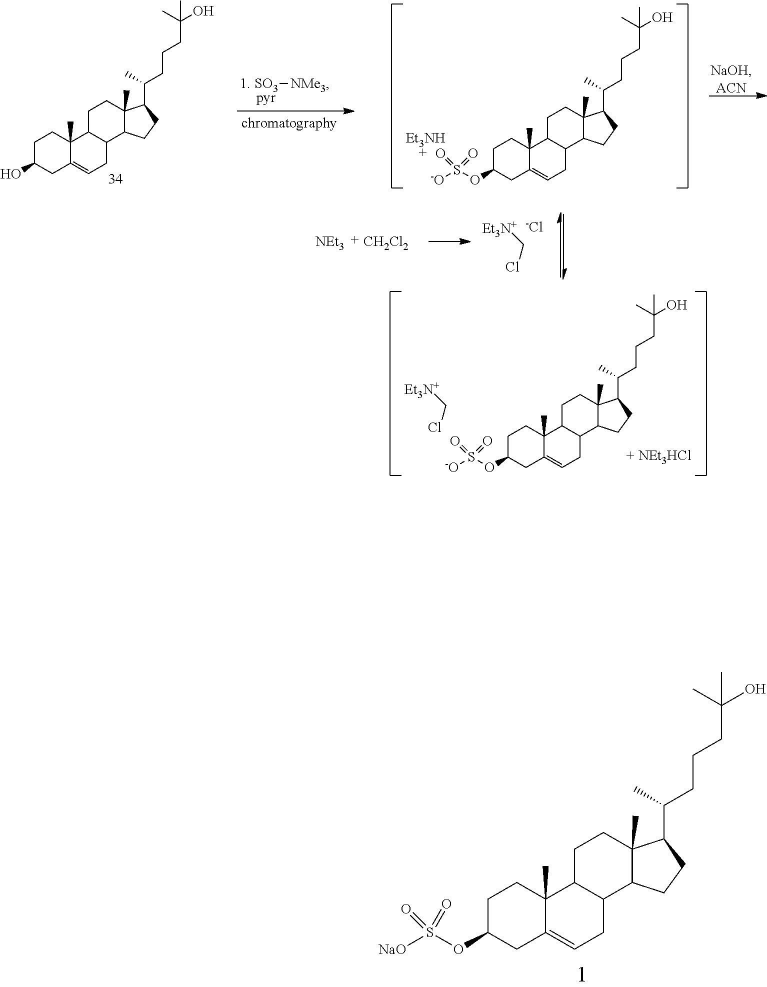

Of the remaining two-thirds of the crude mixture in the carboy, one-third was subjected to chromatography on a fourth column. Pressure was maintained at 0-1 psi during the entire purification. Separation of 3β-triethylamine salt from the 25-sulfate regioisomer was successful: The purity of the 3β-triethylamine salt in the drum was 99.79% by HPLC. Approximately 0.050 kg of 34.1 was isolated from the column (HPLC weight percent assay). The silica gel was cleaned with methanol and regenerated with 1% triethylamine in methylene chloride. The amount of methanol present after regeneration was 0.44% (H NMR area %). No separation occurred for this column. No further purifications were done with the material from the fourth column. Eluent from the third column (˜50 g of the 3β-triethylamine salt) was subjected to the cation exchange beginning with a solvent swap to acetonitrile. After the addition of acetonitrile, water, and 30% sodium hydroxide, the slurry was agitated and then held overnight. Solids were present in the reactor after the post-stir. The mixture was cooled and filtered using a new 8.5″ benchtop filter. The cake was washed with fresh acetonitrile and dried. A sample was analyzed by ¹H NMR and peaks consistent with a quaternary ammonium salt were present in the spectrum. The 25-sulfate regioisomer was present by HPLC.

Eluent from the second column was concentrated under vacuum and dried to a constant weight. Analysis of the yellow powder (540 g) by ¹H NMR showed a ratio (3:1) of the methylene chloride-triethylamine quaternary ammonium salt to the monosulfate compound. All of the crude material (540 g) was charged to a 3 L jacketed reactor. Acetonitrile (1400 g) was charged, and the slurry was heated to 50±5° C. for 30 min. The slurry was cooled to 26° C. and then filtered. The wet cake was analyzed and the ratio of 3)-triethylamine salt/25-sulfate regioisomer to quaternary ammonium salt was ˜1:1. The purified solids and fresh acetonitrile (1400 g) were charged back to the reactor. Water (200 g) was charged after 45 min, agitated for 15 min, and then filtered. The granular powder was dried in a vacuum oven at 40° C. overnight. The filtrate was concentrated to dryness, and the residue was combined with the dried material and both were charged to a 3 L reactor. Acetonitrile (1500 g), 1 N sodium hydroxide (600 g), and 30% sodium hydroxide (40 g) were sequentially charged to the reactor. The slurry was agitated for 48 hours and then filtered at ambient temperature. The cake was dried to a constant weight (173 g) and analyzed by HPLC.

### Example 13A. Purification to Separate the 25-Sulfate Regioisomer from Compound 1

Several solvents were explored to purge the 25-sulfate sodium salt from compound 1. No solids were recovered after dissolving impure compound 1 in polar solvents and then charging anti-solvents (Entries 1 and 2, Table 3). Minimal solids were formed after dissolving the material in methanol and then adding acetonitrile (Entry 3, Table 3). Using 2-propanol (Entry 4, Table 3) and a mixture of methanol and water resulted in a form change, which caused the material to become a thick paste that would not transfer or filter. Trituration of impure compound 1 with ethanol at 40-50° C. was sufficient to purge the majority of the 25-sulfate regioisomer (Entry 6, Table 3). A mixture of compound 1 (1 g) and ethanol (10 mL) was heated to reflux, cooled, and filtered. The isolated material (5500 recovery) was 99.600 pure with the 25-sulfate and 3β, 25-dihydroxycholest-5-ene products reduced to 0.1% and 0.3%, respectively.

TABLE 3
Trituration/recrystallization of Compound 1

| | | Unpurified | | | 3β,25- | |
|---|---|---|---|---|---|---|
| | | Compound 1 | Compound 1 | 25-sulfate | dihydroxycholest- | |
| Sample | Solvent | (g) | Purity | regioisomer | 5-ene | Comment |
| 1 | Ethyl | 0.5 | N/A | N/A | N/A | No Solids |
| | acetate/methanol | | | | | |
| 2 | Dimethyl | 0.5 | N/A | N/A | N/A | No Solid |
| | sulfoxide/iso- | | | | | |
| | propanol | | | | | |
| 3 | Methanol/ | 0.1 | N/A | N/A | N/A | Minimal |
| | acetonitrile | | | | | Solids |
| 4 | Isopropanol | 0.1 | N/A | N/A | N/A | Gel/paste |
| 5 | Methanol/water | 0.5 | 96.3 | 1.8 | 1.9 | Gel/paste |
| 6 | Ethanol | 0.1 | 97.0 | 0.4 | 2.6 | Trituration |
| 7 | Ethanol | 1.0 | 99.6 | 0.1 | 0.3 | 55% recovery |

### Example 13B. Purification of the 393-triethylamine salt

The 3β-triethylamine salt was purified to eliminate methylene chloride due to reactivity with trimethylamine and triethylamine. Purification was achieved using an isocratic solvent system that includes 90% ethyl acetate, 9%˜ methanol and 1% triethylamine.

### Example 13C. Optimization of SO₃NMe₃ Equivalents

The amount of added SO₃NMe₃ complex needed to either completely consume 3β, 25-dihydroxycholest-5-ene or arrive at a point at which the bis-sulfate and unreacted starting material byproducts were minimal was determined. A solution of 3β, 25-dihydroxycholest-5-ene (0.5 g, 1.0S) in pyridine (18.6S) containing triethyl amine (0.5S) was heated to 50′° C. A sample was removed from the reaction every 30 min, which was followed by the addition of the SO₃NMe₃ complex. Following the final charge of the SO₃NMe₃ complex, the vial was allowed to stir at 50° C. for a total of 24 h. (Table 4). Approximately 1.75 equivalents of SO₃NMe₃ complex were sufficient to consume 86.6% of starting material 3β, 25-dihydroxycholest-5-ene (Sample 7, Table 4). 3β, 25-dihydroxycholest-5-ene was completely consumed after 2.5 equivalents of SO₃NMe₃ complex were added. Formation of the bis-sulfate will out-compete mono-sulfation of 3β, 25-dihydroxycholest-5-ene as the reaction progresses. The 3β-tri ethyl amine salt was completely converted to the bis-sulfate after 24 hours.

TABLE 4
Equivalents of sulfur trioxide-trimethylamine complex

| | Equivalents of | 3β-triethylamine | 3β,25- | |
|---|---|---|---|---|
| Sample | SO₃NMe₃ | salt | dihydroxycholest-5-ene | Bis-sulfate |
| 1 | 0.25 | 18.0 | 75.9 | 6.1 |
| 2 | 0.5 | 28.6 | 71.4 | 0.0 |
| 3 | 0.75 | 39.8 | 60.2 | 0.0 |
| 4 | 1.00 | 48.6 | 46.7 | 4.7 |
| 5 | 1.25 | 53.1 | 39.4 | 7.4 |
| 6 | 1.50 | 58.4 | 28.6 | 12.9 |
| 7 | 1.75 | 68.1 | 13.4 | 18.5 |
| 8 | 2.00 | 60.9 | 13.1 | 26.0 |
| 9 | 2.30 | 58.3 | 7.2 | 34.4 |
| 10 | 2.60 | 49.6 | 1.0 | 49.4 |
| 11 | 2.90 | 46.6 | 0.0 | 53.4 |
| 12 | 3.20 | 37.7 | 0.0 | 62.3 |
| 13 | 3.50 | 32.3 | 0.0 | 67.7 |

### Example 14. One-hundred 2ram scale synthesis of 31-25-hydroxycholest-5-ene sulfate (1, as Sodium Salt)

A slurry of 3 Å 25-dihydroxycholest-5-ene (100 g, 1.0S) and triethylamine (0.5S) in pyridine (15.6S) was heated to 50° C. The SO₃NMe₃ complex (1.75 equivalents, 0.6S) was charged in one portion. The mixture was agitated for 5 hours and then analyzed for reaction completion by HPLC (Sample 1-3)₈-tri ethyl amine salt/25-sulfate regioisomer (67.1%); 3β, 25-dihydroxycholest-5-ene (12.2%); bis-sulfate (20.8%)). The jacket was set to 70° C. and the reaction was concentrated to <20% of the initial volume. A sample was removed and analyzed by HPLC for stability (Sample 2-3β-triethylamine salt/25-sulfate regioisomer (60.5%); 3β, 25-dihydroxycholest-5-ene (10.0%); bis-sulfate (29.5%)). The amount of monosulfate decreased from 67.1% to 60.5% during the distillation, while the amount of bisulfate increased ˜9%. The amount of 3β, 25-dihydroxycholest-5-ene did not decrease much during the distillation.

Solids were present in the reactor following a 48 h post stir, and the addition of methanol (0.55) did not dissolve the solids. The crude material (300 g) was subjected to purification by silica gel chromatography eluting with 90% ethyl acetate, 9% methanol, and 1% triethylamine. Silica gel (2.4 kg) was slurried in the eluent and packed to form a 5.25″×28″ column. The crude mixture was transferred to the column, and the purification was carried on over three days. The eluent was collected in 1 L fractions. Fractions 1-7 contained no material detected by TLC; Fractions 8-11 contained pyridine and 3β, 25-dihydroxycholest-5-ene; Fractions 12-20 contained no material detected by TLC; Fractions 21-22 contained an undetermined compound and Fractions 23-59 contained 3β-triethylamine salt/25-sulfate regioisomer.

Approximately 82 g of 3β-triethylamine salt/25-sulfate regioisomer (56.5% yield) was isolated after the column determined by weight percentage analysis. After chromatography, the eluent containing a mixture of 3β-triethylamine salt/25-sulfate regioisomer was concentrated to a slurry and transferred to a 2 liter reactor. The solvent was swapped to acetonitrile, the slurry was cooled to 10° C., and 1 N sodium hydroxide (1.8S, 1 equivalent based on 82 g of 3β-triethylamine salt/25-sulfate regioisomer) was charged over 10 minutes. The slurry was agitated for 1 hour and then filtered. The filtration was very fast, requiring <5 minutes. The solids were dried at 40° C. under vacuum to a constant weight (70 g, 99% yield for the cation exchange). A sample was analyzed by HPLC (Sample 1, Table 5) which indicated that the 25-sulfate regioisomer was present at 5.1%. The white powder (70 g) was transferred to a 2 liter reactor and slurried with ethanol (700 g) at 50° C. for 1 hour. A form change was observed after 30 minutes of stirring by the thickening of the slurry mixture. The slurry was cooled to 10° C., stirred for 1 hour, and then filtered at 10° C. The reactor was rinsed with ethanol (170 g), cooled to 10° C. and then transferred to the filter as a cake wash. The solids were dried to a constant weight (64.6 g, 92.3% recovery) and analyzed by HPLC (Sample 2, Table 5). After trituration, the purity of compound 1 improved to 97.4%, but the 25-sulfate regioisomer was 1.6%. Impure compound 1 (64.6 g, 1.0 S) was slurried in ethanol (581 g, 9S) at 55° C. for 1.5 hours. The slurry was cooled to 10° C. and then filtered. The reactor and cake were rinsed with ethanol (84 g) at 10° C., and the resulting solid was dried at 40° C. under vacuum to a constant weight (60.4 g, ethanol present at 5.9%, 87.9% recovery).

A sample of compound 1 following ethanol trituration was analyzed by HPLC (Sample 3, Table 5). The 25-sulfate regioisomer was purged, but the amount of unknown 1 increased to 0.9%. The purified material (56.8 g) was slurried in acetonitrile (5S) and water (0.9S) at 30° C. for 30 minutes in a 1 liter reactor. The slurry formed stiff peaks during this time, but the paste was easily transferred to the filtration setup using an FMI pump. The reactor and cake were rinsed with fresh acetonitrile (30 g), and the material was dried to a constant weight (54.5 g, 90.2% recovery). Analysis by ¹H NMR showed that ethanol was absent, but water was present at 1.2% by weight. The purity of the final material improved to >99% (Sample 4, Table 5). The unknown impurities at RRT 1.68 and 1.85 were present at 0.6% and 0.2%, respectively. Taking into account the residual water, the final isolated yield of compound 1 in the 100 g demonstration run was 43.2%.

TABLE 5
Purification of Crude Compound 1

| | 25-sulfate | | Unknown 1 | Unknown 2 | Unknown 3 |
|---|---|---|---|---|---|
| | regioisomer (%) | 3β-triethylamine | (%) RRT | (%) RRT | (%) RRT |
| Sample | RRT 0.78 | salt (%) RRT 1.00 | 1.68 | 1.85 | 1.99 |
| 1 | 5.1 | 93.1 | 0.9 | 0.6 | 0.3 |
| 2 | 1.6 | 97.4 | 0.6 | 0.4 | ND |
| 3 | ND | 98.9 | 0.9 | 0.2 | ND |
| 4 | ND | 99.2 | 0.6 | 0.2 | ND |

### Example 15. Azeotropic Removal of Water from 3β, 25-dihydroxycholest-5-ene

A slurry of 3p, 25-dihydroxycholest-5-ene (5 g, LOS) and pyridine (15.6S, 0.016% water, Entry 1, Table 6) was heated to 50° C. A sample of the reaction was removed for water content analysis (0.29%, Entry 2, Table 6). The reaction volume was reduced 50% and sampled for water content (0.042%, Entry 3, Table 6). The amount of pyridine that was collected in the distillate (39 g) was replaced with fresh pyridine in the reactor and sampled again for water (0.027%, Entry 4, Table 6). Once the internal temperature reached 50° C., triethylamine (0.5S) and SO₃NMe₃ (0.6S) were charged to the reactor. The thin white slurry became a clear solution within 15 minutes, and the reaction was agitated at 50° C. A sample was removed at 2 hours and 3 hours for IPC analysis (Entries 1 and 2, Table 7). Only 7.1% 3p, 25-dihydroxycholest-5-ene remained after 2 h. Azeotropically removing the water prior to the addition of SO₃NMe₃ improves the consumption of starting material.

TABLE 6
Water content analysis

| Sample | Comment | % Water |
|---|---|---|
| 1 | Pyridine solvent | 0.016 |
| 2 | Reaction solution before pyridine distillation | 0.285 |
| 3 | Reaction solution after pyridine distillation | 0.042 |
| 4 | Reaction solution after addition of pyridine | 0.027 |

TABLE 7
Reaction completion profile by HPLC

| | | | 3β,25- |
|---|---|---|---|
| | 25-sulfate | 3β-triethylamine salt/25- | dihydroxycholest-5-ene |
| Sample | regioisomer (%) | sulfate regioisomer (%) | (%) |
| 1 | 30.8 | 62.2 | 7.1 |
| 2 | 38.9 | 55.6 | 5.6 |

### Example 16. Ethanol trituration of Crude Compound 1

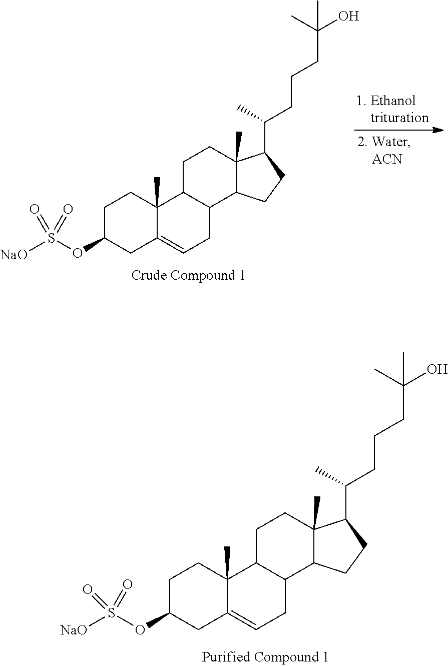

Crude compound 1 was suspended in ethanol and heated to 55° C. and stirred for 1 hour. The slurry mixture is cooled, filtered and washed with ethanol. The resulting cake is dried overnight at 50° C. The cake was charged back into the reactor and suspended in acetonitrile and water. The mixture is heated to 30° C. and stirred for 1 hour. The mixture is then cooled to 15° C., filtered and washed with acetonitrile and water (90:10). The resulting cake is dried for not longer than 24 hours at 50° C. until a constant weight is achieved. Impurity content in purified compound 1 was determined by HPLC. (RRT 0.67<0.05%; RRT 0.77<0.05%; RRT 0.79<0.05%; RRT 0.95<0.05%; RRT 1.13<0.05%; RRT 1.22<0.05%; RRT 1.31<0.05%; RRT 1.95=0.09%; RRT 2.09<0.05%; RRT 2.67<0.05%; RRT 2.75=0.05%; RRT 3.04<0.05%; RRT 3.23=0.09%; RRT 3.64=0.3%; RRT 5.00<0.05%; Total impurities=1.1%.

### Example 17. Identification of Byproducts from Sulfating 25-hydroxy-(3β)-cholest-5-en-3-ol to produce 25-hydroxy-(3)-cholest-5-en-3-sulfate

A composition of 25-hydroxy-(3β)-cholest-5-en-3-ol was sulfated with a sulfur-trioxide pyridine complex in toluene at 23° C. for 1 h to produce 25-hydroxy-(3β)-cholest-5-en-3-sulfate. Compounds formed in a reaction mixture when preparing the 25-hydroxy-(3)-cholest-5-en-3-sulfate product were analyzed by high performance liquid chromatography. Tables 8 and 9 provide the HPLC chromatography conditions. Table 10 lists retention times of compounds identified as being formed in the reaction mixture when sulfating 25-hydroxy-(3)-cholest-5-en-3-ol with a sulfur-trioxide pyridine complex.

TABLE 8
Chromatographic Conditions

| | |
|---|---|
| Column | Aglient Zorbax Eclipse XDB-C8, 3.5 μm |
| | (4.6 × 150) mm |
| Mobile Phase A | 2.5 mM sodium phosphate, pH 2.1 with 0.2% |
| | SDS |
| Mobile Phase B | 10:85:5 MPA:ACN:MeOH |
| Detection | Impurities and calculated purity: 205 nm; |
| | Assay: 220 nm |
| Column Temperature | 45° C. |
| Injection Volume | 25.0 μL |
| Flow Rate | 1.0 mL/min |
| Acquisition Time | 50.0 minutes (including 5.0 min. |
| | re-equilibration) |

TABLE 9
Chromatographic Conditions - Gradient

| Time (minutes) | % A | % B |
|---|---|---|
| 0.0 | 62 | 38 |
| 35 | 0 | 100 |
| 45 | 0 | 100 |
| 45.1 | 62 | 38 |
| 50.0 | 62 | 38 |

TABLE 10
Retention Times

| Compound | Retention Time (min) |
|---|---|
| Pyridine | 3.2 |
| 25-sulfated cholesterol | 6.6 |
| 25-hydroxy-(3β)-cholest-5-en-3-sulfate | 7.7 |
| Unknown Byproduct #1 | 18.3 |
| 25-hydroxy-(3β)-cholest-5-en-3-ol | 26.5 |
| Unknown Byproduct #2 | 37.7 |

### Example 18. Determining Purity of Sulfur Trioxide Pyridine Sulfating Anent

Proton nuclear magnetic resonance spectroscopy (¹H-NMR) was conducted on samples of sulfur trioxide pyridine in a deuterated solvent. Sulfur trioxide pyridine is a colorless solid that can degrade due to the presence of moisture, which can impact the overall yield and reproducibility of sulfating 25-hydroxy-(3β)-cholest-5-en-3-ol. A sample of sulfur trioxide pyridine from three lots (A-C) was dissolved in deuterated acetone ((CD₃)₂C₀) and proton NMR spectra were recorded using a 500 MHz Bruker spectrometer. The NMR spectrum of lot A exhibits a smaller set of peaks at 9.25 ppm than the NMR spectrum of lots. Based on the integrated peak at 9.25 ppm in each spectrum, an impurity level of 21% was calculated for the sulfating agent of lot A, an impurity level of 33% was calculated for the sulfating agent of lot B and an impurity level of 36% was calculated for the sulfating agent of lot C.

### Example 19. Process Parameters for Sulfating 25-hydroxy-(3β)-cholest-5-en-3-ol

A sulfation reaction study to minimize and control the formation of bis-sulfated product 5-cholesten-3β-25-diol-disulfate was conducted.

### Example 19A. Sulfation with particles of 25-hydroxy-(3/3)-cholest-5-en-3-Ol in Reaction Mixture

During the sulfation reaction, it was observed that the 25-hydroxy-(3β)-cholest-5-en-3-sulfate organic cationic salt precipitates as a gel-like solid during the reaction. Some of this colloidal material may be solubilized in the reaction mixture due to its particle size. To minimize this solubility effect, the addition of seed crystals of 25-hydroxy-(3β)-cholest-5-en-3-sulfate organic cationic salt to the reaction to modify the product crystal shape was tested. As the reaction proceeds by charging the sulfur trioxide-pyridine complex, the gel-like solids of 25-hydroxy-(3β)-cholest-5-en-3-sulfate organic cationic salt produced during the sulfation reaction turned into an amorphous slurry with a larger particle size. This allowed for control of the solubility of the generated 25-hydroxy-(33)-cholest-5-en-3-sulfate organic cationic salt in the reaction mixture. This also resulted in minimizing the formation of bis-sulfated product 5-cholesten-3β-25-diol-disulfate in the reaction mixture.

25-hydroxy-(3β)-cholest-5-en-3-ol was dissolved with 2-methyl tetrahydrofuran (30V); and heated to about 35-40° C. The solution was cooled to about 20±5° C. and seed crystals of 25-hydroxy-(3β)-cholest-5-en-3-sulfate organic cationic salt were added. The sulfating agent sulfur-trioxide-pyridine complex was added in four portions held 2 hours apart from each other. Water (2 equivalents) was added to the slurry and held for 1 hour. At this point, agitation was reduced to a minimum vortex deep. Pyridine (2 equivalents) in 2-methyl tetrahydrofuran was added and the slurry was held for 12 hours or longer. Crude 25-hydroxy-(3β)-cholest-5-en-3-sulfate organic cationic salt product was collected by filtration and washed with 2-methyl tetrahydrofuran-pyridine (5%). The presence of bis-sulfated product 5-cholesten-3β-25-diol-disulfate was estimated to be about 2-5% in the crude product.

### Example 19B. Quenching of unreacted Sulfur Trioxide-Pyridine Sulfation Reagent

Quenching excess unreacted sulfur-trioxide pyridine sulfation reagent was evaluated using two equivalents of water and pyridine to keep basic conditions and to avoid hydrolysis of the 25-hydroxy-(3β)-cholest-5-en-3-sulfate organic cationic salt product. In Items 1-3 of Table 11, water and pyridine were added simultaneously and held for one hour; then, the product was isolated by vacuum filtration. In item 4 of Table 11, the holding time was extended to mimic time expansion. To control the competition reactions between reagent hydrolysis and bis-sulfation, reagent hydrolysis was evaluated by adding water and holding it for one hour. This approach maximized excess hydrolysis. Pyridine was then added to minimize product hydrolysis (item5, Table 11). As summarized in Table 11, the addition of water for 1 hour followed by mixing with pyridine overnight afforded the highest yield of the 25-hydroxy-(3β)-cholest-5-en-3-sulfate organic cationic salt product and the lowest amount of bis-sulfated product and desmosterol impurity.

During quenching of excess unreacted sulfation reagent, it was determined that the agitation speed can play a role in competition between formation of bis-sulfated product 5-cholesten-3β-25-diol-disulfate and reagent quench. At high agitation speed, the unquenched sulfur trioxide-pyridine complex agglomerates brake apart, allowing further reaction with the 25-hydroxy-(3β)-cholest-5-en-3-sulfate organic cationic salt product. At slow agitation speed agglomerated complex remains at the bottom of the reactor minimizing this side reaction. Bis-sulfated product 5-cholesten-3β-25-diol-disulfate formation is observed under these reaction conditions in a range of 2-5%. Isolated crude 25-hydroxy-(3β)-cholest-5-en-3-sulfate organic cationic salt product was stable enough for further purification.

TABLE 11
Quenching of unreacted Sulfur Trioxide-Pyridine Sulfation Reagent

| | HPLC area (%) | | | | | |
|---|---|---|---|---|---|---|
| | Bis-sulfated | 25-hydroxy- | | | | |
| | product 5- | (3β)-cholest-5- | | | | |
| | cholesten-3β- | en-3-sulfate | | 25-hydroxy- | | |
| | 25-diol- | organic cationic | Desmosterol | (3β)-cholest- | | |
| Item | disulfate | salt | sulfate | 5-en-3-ol | Stage | Time |
| 1 | 4.4 | 92.9 | 1.329 | 1.26 | IPC | 1 h |
| | 5.0 | 92.0 | 1.46 | 1.54 | Solid | |
| 2 | 1.4 | 97.3 | 0.58 | 0.67 | IPC | 1 h |
| | 2.8 | 94.3 | 0.68 | 2.16 | Solid | |
| 3 | 1.9 | 96.6 | 0.79 | 0.61 | IPC | 1 h |
| | 2.6 | 95.8 | 0.46 | 1.11 | Solid | |
| 4 | 1.1 | 96.4 | 0.77 | 1.67 | IPC | overnight |
| | 3.5 | 95.4 | 0.27 | 0.86 | Solid | |
| 5 | 1.8 | 97.0 | 0.69 | 0.57 | IPC | Water |
| | | | | | | 1 h, |
| | | | | | | pyridine |
| | | | | | | overnight |
| | 2.6 | 96.1 | 0.62 | 0.59 | Solid | |

*IPC—in process control

### Example 19C. Liquid Chromatography and Recrystallization of 25-hydroxy-(3β)-cholest-5-en-3-sulfate organic cationic salt product

The 25-hydroxy-(3β)-cholest-5-en-3-sulfate organic cationic salt product was purified using a plug column employing a silica gel (≥mass equivalent) stationary phase and a mixture of methylene chloride-methanol (85:15) and pyridine (1%) mobile phase. The chromatographic column was prepared with silica gel (5mass-eq)/DCM-Pyridine (1M), with a 1:2 ratio diameter-silica gel. The column was carefully prepared to avoid disturbing the silica gel top layer. Crude 25-hydroxy-(3β)-cholest-5-en-3-sulfate organic cationic salt product was dissolved in methylene chloride-methanol (1:1)-pyridine (1%) (2.4V), the solution charged to the column, and rinsed with methylene chloride-methanol (15%)-Pyridine (1%) (2V). The column was eluted with methylene chloride-methanol (15%)-Pyridine (1%) (˜75V). Samples of about 10V were taken and monitored by thin layer chromatography (mobile phase methylene chloride-methanol 7:3 one drop pyridine and CAM stain). Fractions containing the 25-hydroxy-(3β)-cholest-5-en-3-sulfate organic cationic salt product were combined and the fractions containing bis-sulfated product were excluded.

The 25-hydroxy-(3β)-cholest-5-en-3-sulfate organic cationic salt product was isolated and purified from collected fractions by two different processes:

Isolation and Recrystallization Process (IP)-A. Fractions with the product from the plug column were concentrated under the constant volume technique. The 25-hydroxy-(3β)-cholest-5-en-3-sulfate organic cationic salt product solution was added to the initial constant volume (28V) mixture of 2-methyl tetrahydrofuran-heptane (1:2)—Particle seeds of 25-hydroxy-(30)-cholest-5-en-3-sulfate organic cationic salt product were added while synchronizing distillation and addition. Pressure was maintained between 20-25 in. Hg. Under these conditions, the product precipitated out immediately and remains as a slurry during the distillation. The slurry temperature is adjusted to 20-25° C. and held for a minimum of 1 hour. The product is collected by filtration and rinsed with 2-methyl tetrahydrofuran-heptane (1:2) followed by heptane. The collected material was dried at 30-35° C. under vacuum for 24 hours.

Isolation Process (IP)-B. Fractions with the product from the plug column are concentrated under vacuum to ˜7V. If the solution remained or turned cloudy or solids were observed, methylene chloride was added until a clear solution was obtained. This concentrated 25-hydroxy-(3β)-cholest-5-en-3-sulfate organic cationic salt product solution was added dropwise to a mixture of 2-methyl tetrahydrofuran-heptane (1:3) containing seeds of the 25-hydroxy-(3β)-cholest-5-en-3-sulfate organic cationic salt for about 1 hour to 1.5 hours. The product was rinsed in the container with methylene chloride-methanol (1:1) (0.5V) for 1 hour at 20-25° C. After aging the slurry, the product was collected by filtration and rinsed with 2-methyl tetrahydrofuran-heptane (1:3), followed by heptane. The solids were dried at 30-35° C. under vacuum for 24 hours.

The purity of 25-hydroxy-(3β)-cholest-5-en-3-sulfate organic cationic salt product obtained by each isolation and recrystallization process is summarized in Table 12.

TABLE 12
Purity 25-hydroxy-(3β)-cholest-5-en-3-sulfate organic
cationic salt product after Isolation and recrystallization

| | | HPLC area (%) | | | | | |
|---|---|---|---|---|---|---|---|
| | | Bis-sulfated | 25-hydroxy- | | | | |
| | | product 5- | (3β)-cholest- | | 25-hydroxy- | | |
| | | cholesten-3β- | 5-en-3-sulfate | | (3β)- | | |
| | Scale | 25-diol- | organic | Desmosterol | cholest-5- | Isolation | |
| Step | (g) | disulfate | cationic salt | sulfate | en-3-ol | Process | Yield |
| Sulfation | 430 | 3.89 | 95.23 | 0.40 | 0.43 | — | — |
| Purified | 540 | ND | 99.88 | 0.12 | ND | (IP)-A | 75.1 |
| Sulfation | 40 | 2.28 | 96.83 | 0.75 | 0.14 | — | — |
| Purified | 45 | ND | 99.45 | 0.51 | ND | (IP)-B | 84.0 |

### Example 20. XRPD Indexing

Indexing and structure refinement are computational studies. Within FIG. 4, agreement between the allowed peak positions, marked with bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below FIG. 4 and in Table 2. To confirm the tentative indexing solution, the molecular packing motifs within the crystallographic unit cells must be determined. No attempts at molecular packing were performed.

### Example 21. Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to sample analysis. Samples were analyzed from 30° C. to 350° C. or 30° C. to 250° C., both at 10° C./min.

### Example 22. Proton NMR Spectroscopy

The ¹H-NMR spectrum is, other than a peak at 5.3 ppm, consistent with structure as seen in FIG. 6. There is no evidence of residual solvent. An overlap of a methylene from chlorine at about 3.8 ppm appears to be 1 mol/mol choline. The solution NMR spectra were acquired with an Agilent DD2-400 spectrometer. Samples were prepared by dissolving approximately 2-10 mg of sample in DMSO-d₆ containing TMS. The data acquisition parameters are displayed below in Table 13.

TABLE 13
NMR Parameters
OS: CentOS Linux release 7.6.1810 (Core)
Comp: nmr600-1
Processed on: TopSpin 4.0.8
Acquired on: TopSpin 4.0.8
Current Data Parameters

| | |
|---|---|
| EXPNO | 1 |
| PROCNO | 1 |

F1 - Acquisition Parameters

| INSTRUM | AV4 NEO | |
|---|---|---|
| PROBHD | z149002_002 | |
| SOLVENT | DMSO | |
| TE | 298.1K | |
| RO | 0 | Hz |
| PULPROG | zg | |
| D1 | 10.000 | sec |
| P1 | 12.0 | usec |
| PLW1 | 25.093 | W |
| AQ | 5.000 | sec |
| SWH | 10000.000 | Hz |
| SW | 16.6629 | ppm |
| NS | 16 | |
| DS | 0 | |
| TD | 100000 | |
| NUC1 | 1H | |
| SFO1 | 600.1339636 | MHz |

F1 - Processing parameters

| S1 | 524288 | |
|---|---|---|
| WDW | EM | |
| SSB | 0 | |
| LB | 0.20 | Hz |

| | |
|---|---|
| GB | 0 |

### Example 23. Thermogravimetric Analysis (TGA)

TG analysis was performed using a Mettler-Toledo TGA/DSC3 analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Each sample was heated from ambient temperature to 350° C. at 10° C./min. Although thermograms are plotted by reference temperature (x-axis), results are reported according to sample temperatures.

### Example 24. Transmission XRPD

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 6401) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator)located 240 mm from the specimen and Data Collector software v. 5.5. All images have the instrument labeled as X'Pert PRO MPD regardless of the instrument used.

### Example 25. Reflection Geometry (Samples in Limited Quantity)

To the extent made, XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640f) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5.

### Example 26. Dynamic Vapor Sorption (DVS)

Dynamic vapor sorption data was collected on a Surface Measurement System DVS Intrinsic instrument. The samples were not dried prior to analysis. Sorption and desorption data were collected over a range from 5% to 95% RH in 10% RH increments under a nitrogen purge. The equilibrium criteria used for the analyses were 0.001 dm/dt weight change in 5 minutes with a minimum step time of 30 minutes and maximum equilibration time of 180 minutes with a 3 minute data logging interval. Data were not corrected for the initial moisture content of the sample.

### Example 27. Preparing 25HC3S Triethylammonium

A column containing ˜145 g “wet” Dowex 50WX8 (50-100 mesh, H+ form) was washed with distilled water until the eluate was colorless. A volume of 600 mL 1.5 M triethylammonium hydrochloride in H₂O/methanol (1/1), was treated with 12.6 mL triethylamine (pH now >8.5) and then passed through the column. The resin was then rinsed with methanol until the eluate was neutral (˜200 mL).

41.2 g of 25HC3S sodium was dissolved in 820 mL methanol, and, after sonication, the cloudy solution was filtered through Celite. The filtrate was then passed through the above column and collected in ˜ 200 mL fractions, Complete elution was achieved with an additional 600 mL MeOH. Drying under high vacuum overnight afforded 39 g 25HC3 S triethylammonium as a chunky white solid (81% recovery). Homogenation with a mortar/pestle provided the target as a fine, white powder.

Proton NMR analysis (CDCl₃, d6-DMSO) indicated a quantitative conversion to the triethylammonium salt.

### Example 28. Preparing Crystalline 25HC3S Choline

A suspension of the 25HC3S triethylammonium of Example 27 (4.0 g, 6.9 mmol, 1 eq.) in 70 mL of acetonitrile was treated with 1 eq. choline hydroxide (6.9 mL of a 1 M solution in H₂O) and stirred 2-3 hours at room temperature. The solid was collected and rinsed with acetonitrile, then dried under high vacuum to afford 3.08 g of 25HC3S choline as a white solid (76%).

Proton NMR analysis (d6-DMSO) indicated no residual triethylamine.

### Example A. Solubility of 25HC3S Choline at Room Temperature

### 1. Summary

The solubility of 25HC3S choline in aqueous and organic solvents was determined after one day at room temperature. The aqueous solvents were water, 5% dextrose, 0.9% sodium chloride, Fasted State Simulated Intestinal Fluid (FaSSIF), Fed State Simulated Intestinal Fluid (FeSSIF) and Fasted State Simulated Gastric Fluid (FaSSGF). The organic solvents were methanol, ethanol, isopropanol and acetonitrile.

### 2. Introduction and Purpose

The objective of this study was to determine the solubility of 25HC3S choline at room temperature in 4 organic solvents and 6 aqueous solutions.

### 3. Materials

Materials included the following.
3.1. 0.9% Sodium Chloride: Hospira
3.2. 5% dextrose: Hospira
3.3. Water: DURECT USP purified
3.4. Methanol: EMD
3.5. Ethanol: Pharmco
3.6. Acetonitrile: Honeywell
3.7. Isopropyl alcohol: Honeywell
3.8. FaSSIF, FeSSIF, FaSSGF powder: Biorelevant
3.9. FeSSIF buffer concentrate: Biorelevant
3.10. FaSSIF buffer concentrate: Biorelevant
3.11. FaSSGF buffer concentrate: Biorelevant
3.12. 25HC3S choline used in the study is listed in Table A1.

TABLE A1
25HC3S Choline in Solubility Study

| | Molecular |
|---|---|
| | Weight |
| Compound | (g/mol) |
| 25HC3S choline | 585.88 |

### 4. Study Experimental Procedures

### 4.1 Preparation of Simulated Intestinal and Gastric Fluids

These fluids were prepared per the instruction listed by Biorelevant (Biorelevant.com). Each buffer concentrate was diluted with water and mixed well. FaSSIF, FeSSIF, FaSSGF powder was added to each buffer and mixed to completely dissolve the powder. Buffers were stored at room temperature and used within 48 hours of preparation.

TABLE A2
Preparation of Simulated Intestinal and Gastric Fluids

| | | | Buffer | | | | FaSSIF, FeSSIF, FaSSGF | |
|---|---|---|---|---|---|---|---|---|
| Target | | | Concentrate, g | | Purified Water, g | | Powder, g | |
| Volume, | Simulated | Buffer | Target | Actual | Target | Actual | Target | Actual |
| mL | Fluid | Concentrate | Wt | Wt. | Wt. | Wt. | Wt. | Wt. |
| 450 | FaSSIF | FaSSIF | 18.74 | 18.76 | 432.5 | 432.51 | 1.008 | 1.009 |
| | FeSSIF | FeSSIF | 36.64 | 36.65 | 413.75 | 413.83 | 5.04 | 5.04 |
| | FaSSGF | FaSSGF | 16.55 | 16.58 | 432.85 | 432.86 | 0.027 | 0.028 |

### 4.2 Solubility Sample Preparation

25HC3S choline was weighed in 8 mL or 4 mL glass vials with Teflon coated screw caps. Aliquots of each solvent were added to each sample and vortexed for about one minute until excess solid remained. Samples were placed on shaker at room temperature with 210 motion per minute shaking.

After one day, samples were pulled and checked if excess solid was present. About 1.5 mL aliquot of each sample was centrifuged at 12000 rpm for 12 to 15 minutes. Some samples required 2× centrifugation. Aliquots of the top clear solution of each centrifuged sample in MeOH, EtOH, IPA and ACN were diluted with diluent (water:MeOH 95:5) and assayed by HPLC. Samples of 25HC3S choline in aqueous media were assayed by HPLC without dilution.

Note: some samples did not have excess solid present after 1 day, solubility of drug in these samples is reported as ≥concentration values.

### 4.3 Analytical Testing Procedures

All solubility sample solutions of 25HC3S choline were determined using HPLC. A linear standard curve was constructed using standards with concentration between 11 μg/mL to 1.5 mg/mL as free acid forcing the curve to origin. Solubilities are reported as mg/mL free acid 25HC3S. Detection limit (LOD) of the HPLC method was calculated as 0.0007 mg/mL.

### 5. Results and Discussion

Table A1 lists the relevant information regarding the 25HC3S choline used in the solubility study. Solubility was calculated and reported as the 25HC3S free acid. All solubilities were measured after one day at room temperature.

Table A3 shows solubility results of the 25HC3S choline. The final pH of the sample solutions in aqueous media is listed in Table A4.

TABLE A3
Solubility in Aqueous Solutions and Organic Solvents
at Room Temperature (mg/mL as Free Acid)

| Crystalline 25HC3S Salt | 25HC3S choline |
|---|---|
| Water | 0.107 |
| 0.9% Sodium Chloride | ND |
| 5% Dextrose | 0.093 |
| FaSSGF¹ | 0.008 |
| FaSSIF² | 0.152 |
| FeSSIF³ | 0.827 |
| MeOH⁴ | 17.5 |
| EtOH⁵ | 3.4 |
| IPA⁶ | 0.6 |
| ACN⁷ | 0.5 |

¹FaSSIF = Fasted State Simulated Intestinal Fluid,
²FeSSIF = Fed State Simulated Intestinal Fluid,
³FaSSGF = Fasted State Simulated Gastric Fluid,
⁴MeOH = methanol,
⁵EtOH = ethanol,
⁶IPA = isopropyl alcohol, and
⁷ACN = acetonitrile.
⁸ ND = Not Detected, Detection Limit = 0.7 μg/mL

TABLE A4
pH the Solubility Samples of Crystalline 25HC3S Choline in Aqueous Media¹

| | | 0.9% Sodium | 5% | | | |
|---|---|---|---|---|---|---|
| Compound | Water | Chloride | Dextrose | FaSSGF | FaSSIF | FeSSIF |
| Medium Blank | 6.3 | 5.8 | 4.7 | 1.5, 1.6* | 6.5, 6.5* | 4.9, 5.0* |
| 25HC3S | 8.8 | 8.2 | 7.3 | 1.6 | 6.5 | 5.0 |
| choline | | | | | | |

*Two values were from initial preparation of the simulated fluids and measured at the time of samples measurements.
¹FaSSIF = Fasted State Simulated Intestinal Fluid, FeSSIF = Fed State Simulated Intestinal Fluid, FaSSGF = Fasted State Simulated Gastric Fluid

### Example B. Physical and Chemical Solid-State Stability of 25HC3S Choline

### 1. Summary

The stability of 25HC3S choline was determined after 3 weeks and 5 weeks at 2-8° C. and 80° C.

### 2. Introduction and Purpose

The objective of this study was to determine the solid state physical and chemical stability of 25HC3S choline at 2-8° C. and 80° C.

### 3. Equipment and Materials

Materials are listed in the following.
3.1. Analytical Balance, Mettler MT5
3.2. Sonicator, Branson 8510
3.3. Centrifuge, Eppendorf 5417C
3.4. Refrigerator (2-8° C. storage)
3.5. Incubator (80° C. storage)
3.6. Incubator (60° C. storage)
3.7. HPLC, Agilent 1100
3.8. Methanol: EMD
3.9. Diluent (95% Methanol/5% Water)
3.10. 20 mL Glass Vials with Fluoropolymer Resin/Silicone Septa Lined Screw Caps
3.11. 25HC3S choline used in the study is listed in Table B1.

TABLE B1
25HC3S Choline in Stability Study

| | Molecular |
|---|---|
| | Weight |
| Compound Number / Name | (g/mol) |
| 25HC3S choline | 585.88 |

### 4. Study Experimental Procedures

### 4.1 Stability Sample Preparation for Stability Setup and Testing Schedule

Approximately 25 mg of 25HC3S choline was added to 20 mL glass vials and each powder weight was recorded. Four vials were prepared and stored at 2-8° C. or 80° C. according to Table B2. The samples were scheduled initially to pull for testing at 3 weeks and 6 weeks time points.

TABLE B2
Allocation of Stability Samples for 25HC3S Choline

| Temperature | Number of Vials |
|---|---|
| 80° C. | 2 |
| 2-8° C. | 2 |

### 4.2 Analytical Testing

### 4.2.1 Appearance

For appearance, each unopened vial was visually examined for color, clumping, and flowability.

### 4.2.2 Determination of Assay and Degradation Products

### Preparation of Standard Solutions

Based on the molecular weights, and expected UV response factors used in this study, the expected analytical sample concentrations were within the range of the 25HC3S sodium reference standards (0.75 mg/mL to 1.25 mg/mL as 25HC3S Sodium Salt).

### Preparation of 25HC3S Choline (as Free-Flowing Powder)

Samples were prepared by pipetting 20 mL of 100% methanol into each vial and mixing well, with sonication and vortexing to fully dissolve the samples for HPLC analysis.

### 4.3 HPLC Analysis

HPLC analysis was performed.

### 5. Results and Discussion

Table B1 lists the relevant information regarding the 25HC3S choline used in the stability study. Table B4 shows the stability results of the 25HC3S choline and Table B5 presents stability for the 25HC3S choline with associated comments.

TABLE B4
Stability of 25HC3S Choline at 2-8° C. and 80° C.

| | | | | | % Recovery of 2- | | | | |
|---|---|---|---|---|---|---|---|---|---|
| | | | | | 8° C./3 wk vs | | | | |
| | | Time | | | 60° C./1 wk; 2- | % Impurities | | | |
| | | Point | | Assay | 8° C./5 wk vs | | | ³ 25- | |
| Compound | Condition | (wk) | Appearance | (% w/w) | 80° C./5 wk | ¹ 25-SC | ² SD | HC | Total |
| 25HC3S | 2-8° C. | 3 | White, free | 101.6 | | ND | 0.4 | 0.2 | 1.0 |
| choline | | | flowing powder | | | | | | |
| | | | with clumping | | | | | | |
| | | 5 | White, free | 108.1 | NA | ND | 0.3 | 0.2 | 0.9 |
| | | | flowing powder | | | | | | |
| | | | with clumping | | | | | | |
| | 80° C. | 3 | White, free | 102.4 | 100.8 | ND | 0.4 | 0.4 | 1.2 |
| | | | flowing powder | | | | | | |
| | | | with clumping | | | | | | |
| | | 5 | White, free | 105.2 | 97.3 | ND | 0.8 | 0.2 | 2.2 |
| | | | flowing powder | | | | | | |
| | | | with clumping | | | | | | |

¹ 25-Sulfate-Cholesterol;
² Sulfate Desmosterol;
³ 25-Hydroxy-Cholesterol
ND = Not Detected

TABLE B5
Physical and Chemical Solid-State Stability
of Crystalline 25HC3S Choline

| Compound | Comments |
|---|---|
| 25HC3S Choline | Stable after 5 weeks at 80° C. physically and |
| | chemically. |

### Example C. Measured Powder Flow Properties of 25HC3S Choline

### Background

The pharmaceutical industry relies heavily on the use of powders during the manufacture of dosage forms, especially tablets and capsules. There are numerous references in the pharmaceutical literature, attempting to correlate the various measures of powder flow to manufacturing properties. There are a variety of methods for characterizing powder flow. In addition, while it is clear that no single and simple test method can adequately characterize the flow properties of pharmaceutical powders, certain test methods may be valuable during pharmaceutical development. Two commonly reported methods for testing powder flow are angle of repose and compressibility index or Hausner ratio.

### Angle of Repose

Angle of repose is measured as follows. Powder is poured from a funnel onto a flat surface until a cone of powder is created. The angle of repose is calculated according to the following equation:

$$Angle of respose = arctan ( h / r )$$

$$Where :$$

$$h : height of the cone ( mm )$$

$$r : radius of the cone ( mm )$$

Table C₁, from USP<1174> Powder Flow, describes the powder flow properties correlating to the angles of repose.

TABLE C1
Flow Properties of Powders and the Corresponding Angles
of Repose According to USP<1174> Powder Flow

| | Angle of Repose |
|---|---|
| Flow Property | (degrees) |
| Excellent | 25-30 |
| Good | 31-35 |
| Fair-aid not needed | 36-40 |
| Passable-may hang up | 41-45 |
| Poor-must agitate, vibrate | 46-55 |
| Very poor | 56-65 |
| Very, very poor | >66 |

The lower the angle of repose, the more flowable is a material. Powder with an angle of repose between 25° to 300 will show excellent flow through a hopper, feed frame, die table and die bore smoothly. From 410 to greater than 66°, you may experience powder flow difficulties like rat-holing or bridging within the hopper.

Product Bridging or “arching” and rat-holing are both issues that result in a no-flow condition. Bridging is a case where material that is being discharged or fed forms a bridge or arch over the feed auger or discharge point in a silo cone/hopper. Rat-holing is a condition where the material forms a hole or narrow channel above the feed auger or outlet in a hopper while the remaining material is stationary against the hopper wall. Both of these conditions result in the product not flowing as desired.

### Experimental

The angle of repose was determined by pouring approximately 2 grams of 25HC3S choline through a 7 mm inner diameter plastic tube into the center of a 21.14 mm diameter rubber O-ring, resulting in the formation of a powder cone of fixed diameter. The height of the cone was estimated with calipers.

### Results

Table C₂ shows a summary of the angle of repose measurements for the 25HC3S choline, and their classification according to USP<1174> Powder Flow.

TABLE C2
Angle of Repose for 25HC3S Choline and Classification of Flow
Properties According to USP<1174> Powder Flow

| | | Angle of | |
|---|---|---|---|
| | | Repose | |
| | | (degrees) | |
| Compound | Angle of | Rounded | Flow Property per |
| Number / | Repose | Whole | USP<1174> Powder |
| Name | (degrees) | Number | Flow |
| 25HC3S choline | 34.9 | 35 | Good |

### Compressibility Index and Hausner Ratio

The compressibility index and the closely related Hausner ratio are simple, fast, and popular methods of predicting powder flow characteristics. The compressibility index has been proposed as an indirect measure of bulk density, size and shape, surface area, moisture content, and cohesiveness of materials because all of these can influence the observed compressibility index. The compressibility index and the Hausner ratio are determined by measuring both the bulk volume and the tapped volume of a powder.

Although there are some variations in the method of determining the compressibility index and Hausner ratio, the basic procedure is to measure the unsettled apparent volume, V_{O}, and the final tapped volume, V_{f}, of the powder after tapping the material until no further volume changes occur. The compressibility index and the Hausner ratio are calculated as follows:

$$Compressibility Index = 100 \times [ ( {V}_{O} - {V}_{f} ) / {V}_{O} ]$$

$$Hausner Ratio = ( {V}_{O} / {V}_{f} )$$

Both the Hausner ratio and the compressibility index are empirically established. The Hausner ratio and compressibility index are not absolute properties of a material; its value can vary depending on the methodology used to determine them.

Table C₃, from USP <1174> Powder Flow, describes the flow character of the powder based on the compressibility index and Hausner ratio values.

TABLE C3
Flow Properties of Powders and the Corresponding Compressibility Index
and Hausner Ratios According to USP<1174> Powder Flow

| Compressibility Index (%) | Flow Character | Hausner Ratio |
|---|---|---|
| ≤10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| >38 | Very, very poor | >1.60 |

### Experimental

Powder was carefully poured into a 5 mL glass graduated cylinder (to contain) until it was close to or at 5.0 mL. This initial volume was recorded as Vₒ. The graduated cylinder was tapped on a hard surface for 200 times and the volume was recorded. The cylinder was tapped 100 times more, and the final volume V_{f} was recorded. Typically, the volume after 200 taps was identical to after 300 taps, or within 0.1 mL.

### Results

Table C₄ shows a summary of the Hausner ratio and compressibility index measurements for the 25HC3S choline, and their flow properties classification according to USP<1174> Powder Flow.

TABLE C4
Hausner Ratio and Compressibility Index for 25HC3S
Choline and Classification of Flow Properties According
to USP<1174> Powder Flow

| | | | Scale of |
|---|---|---|---|
| | | | Flowability per |
| | Hausner | Compressibility | USP<1174> |
| Compound | Ratio | Index | (Flow Character) |
| 25HC3S choline | 1.72 | 42 | Very, very poor |

1. 25HC3S choline.

2. The 25HC3S choline of claim 1, wherein the 25HC3S choline is in crystalline form.

3. The crystalline 25HC3S choline of claim 2, having an x-ray powder diffraction pattern comprising a peak at about 3.9° 2θ.

4. The crystalline 25HC3S choline of claim 2, having an x-ray powder diffraction pattern comprising a peak at about 7.8° 2θ.

5. The crystalline 25HC3S choline of claim 2, having an x-ray powder diffraction pattern comprising a peak at about 9.5° 2θ.

6. The crystalline 25HC3S choline of claim 2, having an x-ray powder diffraction pattern comprising a peak at about 10.1° 2θ.

7. The crystalline 25HC3S choline of claim 2, having an x-ray powder diffraction pattern comprising a peak at about 11.0° 2θ.

8. The crystalline 25HC3S choline of claim 2, having an x-ray powder diffraction pattern comprising a peak at about 12.2° 2θ.

9. The crystalline 25HC3S choline of claim 2, having an x-ray powder diffraction pattern comprising a peak at about 13.7° 2θ.

10. The crystalline 25HC3S choline of claim 2, having an x-ray powder diffraction pattern comprising a peak at about 14.7° 2θ.

11-100. (canceled)

101. A process of preparing a salt of 25HC3S comprising preparing a solution of 25HC3S and treating the solution with a choline compound.

102. The process of claim 101, wherein the solution is an organic solution.

103. The process of claim 102, wherein the organic solution comprises acetonitrile.

104. The process of claim 101, wherein the choline compound is choline hydroxide.

105. The process of claim 101, wherein the 25HC3S solution is prepared by dissolving a salt of 25HC3S in an alcohol solvent.

106. The process of claim 105, further comprising preparing a triethylammonium salt of 25HC3 S.

107. The process of claim 106, wherein the triethylammonium salt of 25HC3S is made by the process comprising dissolving 25HC3S sodium in a suitable solvent and treating with a solution comprising triethylammonium hydrochloride and isolating a solid of 25HC3S triethylammonium salt.

108. The process of claim 107, wherein the suitable solvent comprises methanol.

109. The process of claim 107, wherein the solution comprising triethylammonium hydrochloride further comprises triethylamine.

110-111. (canceled)

112. A method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, and conditions related to fat accumulation and inflammation, comprising administering to a patient in need thereof an effective amount of 25HC3S choline.

113-118. (canceled)
